SEQUENCE LISTING (24 sequences)

SEQ ID NO: 1 - skipped

SEQ ID NO: 2 - protein, 5 aa, synthetic construct
GGGGS

SEQ ID NO: 3 - protein, 10 aa, synthetic construct
GGGGSGGGGS

SEQ ID NO: 4 - protein, 15 aa, synthetic construct
GGGGSGGGGSGGGGS

SEQ ID NO: 5 - protein, 25 aa, synthetic construct
GGGGSGGGGSGGGGSGGGGSGGGGS

SEQ ID NO: 6 - protein, 10 aa, synthetic construct
MVPSALTASG

SEQ ID NO: 7 - protein, 114 aa, synthetic construct
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 8 - protein, 162 aa, synthetic construct
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 9 - protein, 65 aa, synthetic construct
ITCPPPMSVEHADIWVKSYSLYSRERYICNSGFKRKAGTSSLTECVLNKATNVAHWTTPSLKCIR

SEQ ID NO: 10 - protein, 64 aa, synthetic construct
TCPPPMSVEHADIWVKSYSLYSRERYICNSGFKRKAGTSSLTECVLNKATNVAHWTTPSLKCIR

SEQ ID NO: 11 - protein, 77 aa, synthetic construct
ITCPPPMSVEHADIWVKSYSLYSRERYICNSGFKRKAGTSSLTECVLNKATNVAHWTTPSLKCIRDPALVHQRPAPP

SEQ ID NO: 12 - protein, 227 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 13 - protein, 227 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 80 aa, synthetic construct
GSPSGESSAPGSSSAESPGPGSESPSGSAPGESSPSGSAPGSPSGESSAPGSPESGSASPGSSSAESPGPGSPSGESSAP

SEQ ID NO: 15 - protein, 163 aa, synthetic construct
GSPSGESSAPGSSSAESPGPGSESPSGSAPGESSPSGSAPGSPSGESSAPGSPESGSASPGSSSAESPGPGSPSGESSAPGSSPSGESSAPGSSSAESPGPGSESPSGSAPGESSPSGSAPGSPSGESSAPGSPESGSASPGSSSAESPGPGSPSGESSAPGS

SEQ ID NO: 16 - protein, 348 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGKLEGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 17 - protein, 321 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKLEGGGGSGGGGSGGGGSGGGGSGGGGSDIITCPPPMSVEHADIWVKSYSLYSRERYICNSGFKRKAGTSSLTECVLNKATNVAHWTTPSLKCIR

SEQ ID NO: 18 - protein, 294 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGSGTCPPPMSVEHADIWVKSYSLYSRERYICNSGFKRKAGTSSLTECVLNKATNVAHWTTPSLKCIR

SEQ ID NO: 19 - protein, 461 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGKLEGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSDIGGGGSGGGGSMVPSALTASGGGGGSGGGGSRGSPSGESSAPGSSSAESPGPGSESPSGSAPGESSPSGSAPGSPSGESSAPGSPESGSASPGSSSAESPGPGSPSGESSAP

SEQ ID NO: 20 - protein, 321 aa, synthetic construct; identical to SEQ ID NO: 17

SEQ ID NO: 21 - protein, 321 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGKLEGGGGSGGGGSGGGGSGGGGSGGGGSDIITCPPPMSVEHADIWVKSYSLYSRERYICNSGFKRKAGTSSLTECVLNKATNVAHWTTPSLKCIR

SEQ ID NO: 22 - protein, 544 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKLEGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSDIGGGGSGGGGSMVPSALTASGGGGGSGGGGSRGSPSGESSAPGSSSAESPGPGSESPSGSAPGESSPSGSAPGSPSGESSAPGSPESGSASPGSSSAESPGPGSPSGESSAPGSSPSGESSAPGSSSAESPGPGSESPSGSAPGESSPSGSAPGSPSGESSAPGSPESGSASPGSSSAESPGPGSPSGESSAPGS

SEQ ID NO: 23 - protein, 311 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGKLEGGGGSGGGGSGGGGSDIITCPPPMSVEHADIWVKSYSLYSRERYICNSGFKRKAGTSSLTECVLNKATNVAHWTTPSLKCIR

SEQ ID NO: 24 - protein, 301 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGKLEGGGGSDIITCPPPMSVEHADIWVKSYSLYSRERYICNSGFKRKAGTSSLTECVLNKATNVAHWTTPSLKCIR

(19) United States
(12) Patent Application Publication
(10) Pub. No.: US 2026/0308234 A1
(43) Pub. Date: Oct. 8, 2026

(54) A NOVEL IL-15R ALPHA FC FUSION PROTEIN AND USES THEREOF

(71) Applicant: STAIDSON BIOPHARMA INC., Richmond, CA (US)

(72) Inventors: Yong YIN, Richmond, CA (US); Wenwu ZHAI, Richmond, CA (US)

(73) Assignee: STAIDSON BIOPHARMA INC., Richmond, CA (US)

(21) Appl. No.: 18/996,824

(22) PCT Filed: Aug. 4, 2023
(86) PCT No.: PCT/US2023/071642

§ 371 Date: Jan. 17, 2025

Related U.S. Application Data
(60) Provisional application No. 63/370,605, filed on Aug. 5, 2022.
(60) Provisional application No. 63/370,606, filed on Aug. 5, 2022.
(60) Provisional application No. 63/370,607, filed on Aug. 5, 2022.
(60) Provisional application No. 63/477,993, filed on Dec. 30, 2022.

(51) Int. Cl. C07K 14/715; A61K 38/00

(52) U.S. Cl. CPC C07K 14/7155; A61K 38/00; C07K 2319/30

(57) ABSTRACT

The present application relates to IL-15Rα and Fc fusion protein, IL-15 and IL-15Rα heterodimeric protein, and isolated nucleic acid molecule encoding any of the fusion protein or the heterodimeric protein, vectors and host cells comprising such nucleic acid molecule, compositions, kits, and articles of manufacture comprising any of the fusion protein or the heterodimeric protein, and methods of making, and using thereof.

This application is a national stage entry of PCT International Application No. PCT/US2023/071642, filed on Aug. 4, 2023, which claims the benefit of U.S. Provisional Application No. 63/370,605, filed on Aug. 5, 2022; 63/370,606, filed on Aug. 5, 2022; 63/370,607, filed on Aug. 5, 2022; and 63/477,993, filed on Dec. 30, 2022, the contents of each of which are incorporated herein by reference in their entirety. The contents of the electronic sequence listing (a novel IL-15R alpha Fc fusion protein and uses thereof SEQ.xml; Size: 25,235 bytes; and Date of Creation: Aug. 5, 2022) is herein incorporated by reference in its entirety.

### FIELD

The present application relates to IL-15Rα (alpha) and Fc fusion protein, IL-15 and IL-15Rα heterodimeric protein including activatable IL-15 prodrugs, and methods of making, and using thereof. This application also relates to cleavage products of said activatable prodrugs and methods of using thereof.

### BACKGROUND

Cytokines are potent immune agonists, which makes them being considered as promising therapeutic agents for oncology. For example, the antitumoral activity of interleukin-15 (IL-15) is currently under investigation and have already been used therapeutically in human.

IL-15 is a member of the four α-helix bundle family with 14-15 kDa molecular weight and 114 amino acids (Fehniger T A, Caligiuri M A. Interleukin 15: biology and relevance to human disease. Blood. 2001; 97:14-32), and is produced by mononuclear phagocytes and other cells of the immune system. IL-15 is essential for natural killer cells (NK), natural killer T cells (NKT), and memory CD8+ T cells development and function.

However, cytokines probed to have a very narrow therapeutic window and a short serum half-life. Consequently, therapeutic administration of cytokines produced undesirable systemic effects and toxicities. These were aggravated by the need to administer large quantities of cytokines to achieve the desired levels of cytokines at the intended site of cytokines action (e.g., a tumor). There is an urgent need to find an approach which can increase the in vivo half-life, and promote, reduce the side effects and toxicities or enhance the biological activity of IL-15 in vivo.

Recently, it was found that the complex formed by IL-15 and its receptor IL-15Rα can significantly enhance the biological activity of IL-15. Studies indicated that the complex formed by IL-15 and soluble IL-15Rα receptor is significantly superior to IL-15 alone in stimulating the proliferation of memory CD8+ T lymphocytes and NT/NKT cells. IL-15/IL-15Rα complex is stronger than IL-15 alone in stimulating proliferation of memory CD8+ T cells and in maintaining their survival. The mechanism may be related to cis presentation.

Though the researchers have engaged in research related to IL-15 immunotherapy including IL-15 and Fc fusion protein, IL-15Rα and IL-15 fusion protein or IL-15, IL-15Rα and Fc heterodimeric protein, but due to the IL-15 has the disadvantage of short in vivo half-life and is likely to cause systemic immune side effects, there is still a need for increased the biological activity of IL-15 in vivo.

Surprisingly, the inventor found that the length of the linker between Fc domain and IL-15Rα sushi domain in certain range does affect the biological activity of the IL-15 and IL-15Rα heterodimeric protein, based on which the present application was provided.

### BRIEF SUMMARY

The present application relates to IL-15Rα and Fc fusion protein, IL-15 and IL-15Rα heterodimeric protein including activatable IL-15 prodrugs, and methods of making, and using thereof. The IL-15 and IL-15Rα heterodimeric protein of the present application shows good activity, stability, prolonged in vivo half-life.

One aspect of the present application provides an isolated fusion protein, wherein the fusion protein comprises: an IL-15Rα or a functional fragment thereof linked to an Fc domain through a peptide linker.

In some embodiments, the isolated fusion protein provided herein, wherein the peptide linker comprises 3 to 40 amino acid residues. In some embodiments, the peptide linker comprises 3 to 25 amino acids residues. In some embodiments, the isolated fusion protein provided herein, wherein the peptide linker comprises 20 to 25 amino acid residues. In some embodiments, the peptide linker comprises 25 amino acids residues.

In some embodiments, the peptide linker is rich in G and S amino acid residues. In some preferred embodiments, the peptide linker consists of G and S amino acid residues.

In some embodiments, the isolated fusion protein provided herein, wherein the IL-15Rα or a functional fragment thereof is linked to the N-terminus or C-terminus of the Fc domain.

In some embodiments, the isolated fusion protein provided herein, wherein the fusion protein further comprises an IL-15 cytokine.

In some embodiments, the isolated fusion protein provided herein, wherein the IL-15 cytokine is linked to the N-terminus or C-terminus of the Fc domain.

In some embodiments, the isolated fusion protein provided herein, wherein the IL-15 cytokine is linked to the N-terminus or C-terminus of IL-15Rα or a functional fragment thereof.

Another aspect of the present application provides an isolated heterodimeric protein comprising two polypeptide chains, wherein one polypeptide chain comprises any one of the isolated fusion proteins described herein, and the other polypeptide chain comprises an IL-15 cytokine and/or a second Fc domain.

In some embodiments, the isolated heterodimeric protein provided herein, wherein the IL-15Rα or a functional fragment thereof and the IL-15 cytokine is linked to the N-terminus or C-terminus of the Fc domain.

In some embodiments, the isolated heterodimeric protein provided herein, wherein the Fc domain is selected from the group consisting of a human IgG1 Fc domain, a human IgG2 Fc domain, a human IgG3 Fc domain, a human IgG4 Fc domain, an IgA Fc domain, an IgD Fc domain, an IgE Fc domain, and an IgM Fc domain; optionally, the Fc domain is a human IgG1 Fc domain.

In some embodiments, the isolated heterodimeric protein provided herein, wherein the Fc domain is a human IgG1 Fc domain having L234A and L235A mutations, according to EU Numbering system.

In some embodiments, the isolated heterodimeric protein provided herein, wherein the Fc domains comprises knobs-into-holes mutations (Fc knob and Fc hole).

In some embodiments, the isolated heterodimeric protein provided herein, wherein the Fc knob is linked to the IL-15 cytokine, and the Fc hole is linked to the IL-15Rα or a functional fragment; or the Fc knob is linked to the IL-15Rα or a functional fragment, and the Fc hole is linked to the IL-15 cytokine.

In some embodiments, the isolated heterodimeric protein provided herein, wherein the Fe knob comprises a T366W mutation in the Fc domain, and the Fe hole comprises T366S, L368A, and Y407V mutations in the Fc domain, according to EU Numbering system.

In some embodiments, the isolated heterodimeric protein provided herein, wherein the Fe knob further comprises S354C mutation, and the Fe hole further comprises Y349C mutation, according to EU Numbering system.

In some embodiments, the isolated fusion protein or heterodimeric protein provided herein, wherein the IL-15Rα or a functional fragment is selected from an extracellular region of IL-15Rα or a sushi domain or functional analogs.

In some embodiments, the isolated fusion protein or heterodimeric protein provided herein, wherein the IL-15Rα or a functional fragment comprises the amino acid sequence of any one of SEQ ID NOs: 9-11, or a variant thereof having at least about 90% (such as at least about any one of 91%, 92%, 93%, 94%, 95%, 96%, 97%, 98%, or 99%) sequence identity to the amino acid sequence of any one of SEQ ID NOs: 9-11.

In some embodiments, the isolated fusion protein or heterodimeric protein provided herein, wherein the IL-15 cytokine comprises the amino acid sequence of any one of SEQ ID NOs: 7-8, or a variant thereof having at least about 90% (such as at least about any one of 91%, 92%, 93%, 94%, 95%, 96%, 97%, 98%, or 99%) sequence identity to the amino acid sequence of any one of SEQ ID NOs: 7-8.

In some embodiments, the isolated fusion protein or heterodimeric protein provided herein, wherein the peptide linker comprises the amino acid sequence of any one of SEQ ID NOs: 1-5, or a variant thereof having at least about 90% (such as at least about any one of 91%, 92%, 93%, 94%, 95%, 96%, 97%, 98%, or 99%) sequence identity to the amino acid sequences of any one of SEQ ID NOs: 1-5.

In some embodiments, the isolated fusion protein or heterodimeric protein provided herein, wherein the isolated fusion protein or heterodimeric protein further comprise cleavable moiety (CM) and masking polypeptide (MP), and wherein the masking polypeptide (MP) is linked to the fusion protein or the heterodimeric protein through the cleavable moiety (CM).

In some embodiments, the isolated fusion protein or heterodimeric protein provided herein, wherein the masking polypeptide (MP) comprises the amino acid sequence of any one of SEQ ID NOs: 14-15, or a variant thereof having at least about 90% (such as at least about any one of 91%, 92%, 93%, 94%, 95%, 96%, 97%, 98%, or 99%) sequence identity to the amino acid sequence of any one of SEQ ID NOs: 14-15.

In some embodiments, the isolated fusion protein or heterodimeric protein provided herein, wherein cleavable moiety (CM) comprises the amino acid sequence of SEQ ID NO: 6, or a variant thereof having at least about 90% (such as at least about any one of 91%, 92%, 93%, 94%, 95%, 96%, 97%, 98%, or 99%) sequence identity to the amino acid sequence of SEQ ID NO: 6.

In some embodiments, the isolated fusion protein or heterodimeric protein provided herein comprising two polypeptide chains, wherein one polypeptide chain comprises the amino acid sequence of SEQ ID NO: 16, or a variant thereof having at least about 80% (such as at least about any one of 80%, 85%, 90%, 95%, 96%, 97%, 98%, or 99%) sequence identity to the amino acid sequence of SEQ ID NO: 16; and the other polypeptide chain comprises the amino acid sequence of SEQ ID NO: 17, or a variant thereof having at least about 80% (such as at least about any one of 80%, 85%, 90%, 95%, 96%, 97%, 98%, or 99%) sequence identity to the amino acid sequence of SEQ ID NO: 17.

In some embodiments, the isolated fusion protein or heterodimeric protein provided herein comprising two polypeptide chains, wherein one polypeptide chain comprises the amino acid sequence of SEQ ID NO: 16, or a variant thereof having at least about 80% (such as at least about any one of 80%, 85%, 90%, 95%, 96%, 97%, 98%, or 99%) sequence identity to the amino acid sequence of SEQ ID NO: 16; and the other polypeptide chain comprises the amino acid sequence of SEQ ID NO: 18, or a variant thereof having at least about 80% (such as at least about any one of 80%, 85%, 90%, 95%, 96%, 97%, 98%, or 99%) sequence identity to the amino acid sequence of SEQ ID NO: 18.

In some embodiments, the isolated fusion protein or heterodimeric protein provided herein comprising two polypeptide chains, wherein one polypeptide chain comprises the amino acid sequence of SEQ ID NO: 19, or a variant thereof having at least about 80% (such as at least about any one of 80%, 85%, 90%, 95%, 96%, 97%, 98%, or 99%) sequence identity to the amino acid sequence of SEQ ID NO: 19; and the other polypeptide chain comprises the amino acid sequence of SEQ ID NO: 20, or a variant thereof having at least about 80% (such as at least about any one of 80%, 85%, 90%, 95%, 96%, 97%, 98%, or 99%) sequence identity to the amino acid sequence of SEQ ID NO: 20.

In some embodiments, the isolated fusion protein or heterodimeric protein provided herein comprising two polypeptide chains, wherein one polypeptide chain comprises the amino acid sequence of SEQ ID NO: 19, or a variant thereof having at least about 80% (such as at least about any one of 80%, 85%, 90%, 95%, 96%, 97%, 98%, or 99%) sequence identity to the amino acid sequence of SEQ ID NO: 19; and the other polypeptide chain comprises the amino acid sequence of SEQ ID NO: 18, or a variant thereof having at least about 80% (such as at least about any one of 80%, 85%, 90%, 95%, 96%, 97%, 98%, or 99%) sequence identity to the amino acid sequence of SEQ ID NO: 18.

In some embodiments, the isolated fusion protein or heterodimeric protein provided herein comprising two polypeptide chains, wherein one polypeptide chain comprises the amino acid sequence of SEQ ID NO: 21, or a variant thereof having at least about 80% (such as at least about any one of 80%, 85%, 90%, 95%, 96%, 97%, 98%, or 99%) sequence identity to the amino acid sequence of SEQ ID NO: 21; and the other polypeptide chain comprises the amino acid sequence of SEQ ID NO: 22, or a variant thereof having at least about 80% (such as at least about any one of 80%, 85%, 90%, 95%, 96%, 97%, 98%, or 99%) sequence identity to the amino acid sequence of SEQ ID NO: 22.

In some embodiments, the isolated fusion protein or heterodimeric protein provided herein comprising two polypeptide chains, wherein one polypeptide chain comprises the amino acid sequence of SEQ ID NO: 23, or a variant thereof having at least about 80% (such as at least about any one of 80%, 85%, 90%, 95%, 96%, 97%, 98%, or 99%) sequence identity to the amino acid sequence of SEQ ID NO: 23; and the other polypeptide chain comprises the amino acid sequence of SEQ ID NO: 22, or a variant thereof having at least about 80% (such as at least about any one of 80%, 85%, 90%, 95%, 96%, 97%, 98%, or 99%) sequence identity to the amino acid sequence of SEQ ID NO: 22.

In some embodiments, the isolated fusion protein or heterodimeric protein provided herein comprising two polypeptide chains, wherein one polypeptide chain comprises the amino acid sequence of SEQ ID NO: 24, or a variant thereof having at least about 80% (such as at least about any one of 80%, 85%, 90%, 95%, 96%, 97%, 98%, or 99%) sequence identity to the amino acid sequence of SEQ ID NO: 24; and the other polypeptide chain comprises the amino acid sequence of SEQ ID NO: 22, or a variant thereof having at least about 80% (such as at least about any one of 80%, 85%, 90%, 95%, 96%, 97%, 98%, or 99%) sequence identity to the amino acid sequence of SEQ ID NO: 22.

In another aspect of the present application provides a method of improving the activity of any of the fusion protein or the heterodimeric protein comprising IL-15Rα or a functional fragment thereof and Fc domain, comprising set the peptide linker connecting the IL-15Rα or a functional fragment thereof and the Fc domain comprising more than 15 and less than 40 amino acid residues. In some embodiments, the peptide linker comprises 20 to 25 amino acid residues. In some embodiments, the peptide linker comprises 25 amino acid residues.

Also provided are isolated nucleic acid molecule encoding any of the fusion protein or the heterodimeric protein described herein, vectors comprising such nucleic acid molecule, host cell (e.g., CHO cells, HEK 293 cells, Hela cells, or COS cells) comprising such nucleic acids or vectors, compositions (e.g., pharmaceutical compositions), kits, and articles of manufacture comprising any of the fusion protein or the heterodimeric protein described herein.

Methods of treating a disease (e.g., a tumor) in an individual (e.g., human) using any of the fusion protein or the heterodimeric protein provided herein or pharmaceutical compositions thereof are also provided.

### BRIEF DESCRIPTION OF THE DRAWINGS

FIG. 1 depicts an exemplary IL-15 and IL-15Rα and heterodimeric protein, showing that in one monomer, an IL-15Rα_sushi domain is linked to the C-terminus of one Fc domain through a peptide linker, in the other monomer, an IL-15 is linked to the C-terminus of the other Fc domain.

FIG. 2 depicts an exemplary IL-15 prodrug with an Fc domain as a half-life extension moiety, showing that in one monomer, an IL-15Rα_sushi domain is linked to the C-terminus of one Fc domain through a non-cleavable linker, in the other monomer, an IL-15 is linked to the C-terminus of the other Fc domain, and the masking polypeptide (MP) is linked to the IL-15 through a cleavable moiety (CM).

FIG. 3 depicts the results of non-reduced and reduced SDS-PAGE gels analyzing the purity of exemplary IL-15 and IL-15Rα and heterodimeric protein SB1902-C1.

FIG. 4 depicts the results of SEC-HPLC analyzing the homogeneity of exemplary IL-15 and IL-15Rα and heterodimeric protein SB1902-C1.

FIG. 5 shows the binding of the exemplary IL-15 and IL-15Rα heterodimeric protein SB1902-C1 and the exemplary IL-15 prodrug SB1902-C2 to IL-2/IL-15Rβγ receptor.

FIG. 6 depicts the results of the exemplary IL-15 and IL-15Rα heterodimeric protein SB1902-C1 and SB1902-C1-variant1 in CD8+ T cell activation assay.

FIG. 7 depict the results of the exemplary IL-15 prodrug SB1902-C2 and SB1902-C2-variant0 in CD8+ T cell activation assay.

FIG. 8 depicts the results of the exemplary IL-15 prodrug SB1902-C9-variant1, SB1902-C9-variant2 and SB1902-C9-variant3 in CD8+ T cell activation assay.

FIGS. 9-10 show the results of the treatment to WEHI-164 tumor animals with IgG1 isotype control antibody MOPC-21 (FIG. 9) or an IL-15 and IL-15Rα heterodimeric protein SB1902-C1 (FIG. 10) at a dose of 3 mg/kg. The tumor growth in SB19020-C1 treated animals was inhibited compared to the tumor growth in animals treated with isotype control antibody.

### DETAILED DESCRIPTION

Disclosed herein are IL-15Rα and Fc fusion protein, IL-15 and IL-15Rα heterodimeric protein, including activatable IL-15 prodrugs. The IL-15 and IL-15Rα heterodimeric protein with long half-life and better activity. The prodrugs overcome the toxicity that have severely limited the clinical use of the IL-15 cytokine.

### Definitions

The practice of the present application will employ, unless indicated specifically to the contrary, conventional methods of virology, immunology, microbiology, molecular biology, and recombinant DNA techniques within the skills of the art, many of which are described below for illustration. Such techniques are explained fully in the literature. See, e.g., Current Protocols in Molecular Biology or Current Protocols in Immunology, John Wiley & Sons, New York, N.Y. (2009); Ausubel et al., Short Protocols in Molecular Biology, 3rd ed., John Wiley & Sons, 1995; Sambrook and Russell, Molecular Cloning: A Laboratory Manual (3rd Edition, 2001); Maniatis et al., Molecular Cloning: A Laboratory Manual (1982); DNA Cloning: A Practical Approach, vol. I&II (D. Glover, ed.); Oligonucleotide Synthesis (N. Gait, ed., 1984); Nucleic Acid Hybridization (B. Hames & S. Higgins, eds., 1985); Transcription and Translation (B. Hames & S. Higgins, eds., 1984); Animal Cell Culture (R. Freshney, ed., 1986); Perbal, A Practical Guide to Molecular Cloning (1984) and other like references.

As used herein, “treatment” or “treating” is an approach for obtaining beneficial or desired results including clinical results. For purposes of this application, beneficial or desired results including clinical results, but are not limited to, one or more of the following: alleviating one or more symptoms resulting from the disease, diminishing the extent of the disease, stabilizing the disease (e.g., preventing or delaying the worsening of the disease), preventing or delaying the spread of the disease, preventing or delaying the recurrence of the disease, delaying or slowing the progression of the disease, ameliorating the disease state, providing a remission (partial or total) of the disease, decreasing the dose of one or more other medications required to treat the disease, delaying the progression of the disease, increasing the quality of life, and/or prolonging survival. Also encompassed by “treatment” is a reduction of a pathological consequence of the disease. The methods of the application contemplate any one or more of these aspects of treatment. For example, an individual is successfully “treated” if one or more symptoms associated with the disease are mitigated or eliminated, including, but are not limited to, decreasing symptoms resulting from the disease, increasing the quality of life of those suffering from the disease, decreasing the dose of other medications required to treat the disease, and/or prolonging survival of individuals.

The term “prevent,” and similar words such as “prevented,” “preventing” etc., indicate an approach for preventing, inhibiting, or reducing the likelihood of the occurrence or recurrence of, a disease or condition. It also refers to delaying the occurrence or recurrence of a disease or condition or delaying the occurrence or recurrence of the symptoms of a disease or condition. As used herein, “prevention” and similar words also include reducing the intensity, effect, symptoms, and/or burden of a disease or condition prior to recurrence of the disease or condition.

As used herein, “delaying” the development of a disease means to defer, hinder, slow, retard, stabilize, and/or postpone development of the disease. This delay can be of varying lengths of time, depending on the history of the disease and/or individual being treated. A method that “delays” development of a disease is a method that reduces probability of disease development in a given time frame and/or reduces the extent of the disease in a given time frame, when compared to not using the method. Such comparisons are typically based on clinical studies, using a statistically significant number of individuals.

The term “effective amount” used herein refers to an amount of an agent or a combination of agents, sufficient to treat a specified disorder, condition, or disease such as ameliorate, palliate, lessen, and/or delay one or more of its symptoms. In some embodiments, an effective amount is an amount sufficient to delay disease development. In some embodiments, an effective amount is an amount sufficient to prevent or delay disease occurrence or recurrence. An effective amount can be administered in one or more administrations. In the case of a disease such as cancer, an effective amount may be an amount sufficient to delay cancer development or progression (e.g., decrease tumor growth rate, and/or delay or prevent tumor angiogenesis, metastasis, or infiltration of cancer cells into peripheral organs), reduce the number of epithelioid cells, cause cancer regression (e.g., shrink or eradicate a tumor), and/or prevent or delay cancer occurrence or recurrence. An effective amount can be administered in one or more administrations.

As used herein, an “individual” or a “subject” refers to a mammal, including, but not limited to, human, bovine, horse, feline, canine, rodent, or primate. In some embodiments, the individual is a human.

Antibodies are assigned to classes based on the amino acid sequence of the constant region of their heavy chain. The five major classes or isotypes of antibodies are IgA, IgD, IgE, IgG, and IgM, which are characterized by the presence of α, δ, ε, γ, and μ heavy chains, respectively. Several of the major antibody classes are divided into subclasses such as IgG1 (γ1 heavy chain), IgG2 (γ2 heavy chain), IgG3 (γ3 heavy chain), IgG4 (γ4 heavy chain), IgAQ1 (α1 heavy chain), or IgA2 (α2 heavy chain).

The term “Fc,” “Fc region,” “fragment crystallizable region,” “Fc domain,” or “Fc moiety” herein is used to define a C-terminal region of an immunoglobulin heavy chain, including native-sequence Fc regions and variant Fc regions. Although the boundaries of the Fc region of an immunoglobulin heavy chain might vary, the human IgG heavy chain Fc region is usually defined to stretch from an amino acid residue at position Cys226, or from Pro230 to the carboxyl-terminus thereof. The C-terminal lysine (residue 447 according to the EU numbering system) of the Fc region may be removed, for example, during production or purification of the protein, or by recombinantly engineering the nucleic acid encoding the protein. Suitable native-sequence Fc regions for use in the constructs described herein include human IgG1, IgG2 (IgG2A, IgG2B), IgG3, and IgG4.

The term IgG “isotype” or “subclass” as used herein is meant any of the subclasses of immunoglobulins defined by the chemical and antigenic characteristics of their constant regions. There are five major classes of immunoglobulins: IgA, IgD, IgE, IgG, and IgM, and several of these may be further divided into subclasses (isotypes), e.g., IgG1, IgG2, IgG3, IgG4, IgAQ1, and IgA2. The heavy chain constant domains that correspond to the different classes of immunoglobulins are called α, γ, ε, γ, and μ, respectively. The subunit structures and three-dimensional configurations of different classes of immunoglobulins are well known and described generally in, for example, Abbas et al. Cellular and Mol. Immunology, 4th ed. (W.B. Saunders, Co., 2000).

“Fe receptor” or “FcR” describes a receptor that binds the Fe region of an Fe-containing construct (e.g., antibody or protein containing Fc region, referred to as Fc fusion protein hereafter). The preferred FcR is a native sequence of human FcR. Moreover, a preferred FcR is one that binds an IgG antibody (a gamma receptor) and includes receptors of the FcγRI, FcγRII, and FcγRIII subclasses, including allelic variants and alternatively spliced forms of these receptors, FcγRII receptors include FcγRIIA (an “activating receptor”) and FcγRIIB (an “inhibiting receptor”), which have similar amino acid sequences that differ primarily in the 10 cytoplasmic domains thereof. Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain. (See M. Daëron, Annu. Rev. Immunol. 15:203-234 (1997). FcRs are reviewed in Ravetch and Kinet, Annu. Rev. Immunol. 9: 457-92 (1991); Capel et al., Immunomethods 4: 25-34 (1994); and de Haas et al., J. Lab. Clin. Med. 126: 330-41 (1995). Other FcRs, including those to be identified in the future, are encompassed by the term “FcR” herein.

The term “Fc receptor” or “FcR” also includes the neonatal receptor, FcRn, which is responsible for the transfer of maternal IgGs to the fetus. Guyer et al., J. Immunol. 117: 587 (1976) and Kim et al., J. Immunol. 24: 249 (1994). Methods of measuring binding to FcRn are known (see, e.g., Ghetie and Ward, Immunol. Today 18: (12): 592-8 (1997); Ghetie et al., Nature Biotechnology 15 (7): 637-40 (1997); Hinton et al., J. Biol. Chem. 279 (8): 6213-6 (2004); WO 2004/92219 (Hinton et al.). Binding to FcRn in vivo and serum half-life of human FcRn high-affinity binding polypeptides can be assayed, e.g., in transgenic mice or transfected human cell lines expressing human FcRn, or in primates to which the polypeptides having a variant Fc region are administered. WO 2004/42072 (Presta) describes antibody variants which improved or diminished binding to FcRs. See also, e.g., Shields et al., J. Biol. Chem. 9(2): 6591-6604 (2001).

“Antibody effector functions” refer to those biological activities attributable to the Fc region (a native sequence Fc region or amino acid sequence variant Fc region) of an Fc-containing construct (e.g., antibody or Fc fusion protein), and vary with Fc isotype. Examples of antibody effector functions include C1q binding and complement dependent cytotoxicity (CDC); Fc receptor binding; antibody-dependent cell-mediated cytotoxicity (ADCC); phagocytosis; down regulation of cell surface receptors (e.g., B cell receptors); and B cell activation. “Reduced or minimized” antibody effector function means that which is reduced by at least 50% (alternatively 60%, 65%, 70%, 75%, 80%, 85%, 90%, 95%, 96%, 97%, 98%, or 99%) from the wild type or unmodified Fc-containing construct (e.g., antibody or Fc fusion protein). The determination of antibody effector function is readily determinable and measurable by one of ordinary skill in the art. In a preferred embodiment, the antibody effector functions of complement binding, complement dependent cytotoxicity and antibody dependent cytotoxicity are affected. In some embodiments, effector function is eliminated through a mutation in the constant region that eliminated glycosylation, e.g., “effectless mutation.” In some embodiments, the effectless mutation is an N297A or DANA mutation (D265A+N297A) in the C_{H}2 region. Shields et al., J. Biol. Chem. 276 (9): 6591-6604 (2001). Alternatively, additional mutations resulting in reduced or eliminated effector function include K322A and L234A/L235A (LALA). Alternatively, effector function can be reduced or eliminated through production techniques, such as expression in host cells that do not glycosylate (e.g., E. coli.) or in which result in an altered glycosylation pattern that is ineffective or less effective at promoting effector function (e.g., Shinkawa et al., J. Biol. Chem. 278(5): 3466-3473 (2003).

“Antibody-dependent cell-mediated cytotoxicity” or ADCC refers to a form of cytotoxicity in which secreted Ig (or Ligand-Fc construct) bound onto Fc receptors (FcRs) present on certain cytotoxic cells (e.g., natural killer (NK) cells, neutrophils, and macrophages) enable these cytotoxic effector cells to bind specifically to an antigen-bearing (or ligand receptor-bearing) target cell and subsequently kill the target cell with cytotoxins. The antibodies (or Fc-containing constructs) “arm” the cytotoxic cells and are required for killing the target cell by this mechanism. The primary cells for mediating ADCC, NK cells, express FcγRIII only, whereas monocytes express FcγRI, FcγRII, and FcγRIII. Fc expression on hematopoietic cells is summarized in Table 2 on page 464 of Ravetch and Kinet, Annu. Rev. Immunol. 9: 457-92 (1991). To assess ADCC activity of a molecule of interest, an in vitro ADCC assay, such as that described in U.S. Pat. No. 5,500,362 or 5,821,337 may be performed. Useful effector cells for such assays include peripheral blood mononuclear cells (PBMC) and natural killer (NK) cells. Alternatively, or additionally, ADCC activity of the molecule of interest may be assessed in vivo, e.g., in an animal model such as that disclosed in Clynes et al., PNAS USA 95:652-656 (1998).

“Complement dependent cytotoxicity” or “CDC” refers to the lysis of a target cell in the presence of complement. Activation of the classical complement pathway is initiated by the binding of the first component of the complement system (C1q) to Fc-containing constructs (of the appropriate subclass) which are bound to their cognate receptor through the ligand fused to Fc. To assess complement activation, a CDC assay, e.g., as described in Gazzano-Santoro et al., J. Immunol. Methods 202: 163 (1996), may be performed. Antibody variants with altered Fc region amino acid sequences and increased or decreased C1q binding capability are described in U.S. Pat. No. 6,194,551B1 and WO99/51642. The contents of those patent publications are specifically incorporated herein by reference. See, also, Idusogie et al. J. Immunol. 164: 4178-4184 (2000).

As used herein, the term “specifically binds,” “specifically recognizes,” or is “specific for” refers to measurable and reproducible interactions such as binding between a ligand and a receptor, which is determinative of the presence of the ligand in the presence of a heterogeneous population of molecules including biological molecules. For example, a ligand that specifically binds a receptor is a ligand that binds this receptor with greater affinity, avidity, more readily, and/or with greater duration than it binds other receptors. In some embodiments, the extent of binding of a ligand to an unrelated receptor is less than about 10% of the binding of the ligand to the target receptor as measured, e.g., by a radioimmunoassay (RIA). In some embodiments, a ligand that specifically binds a target receptor has an equilibrium dissociation constant (Kd) of ≤10⁻⁵ M, ≤10⁻⁶ M, ≤10⁻⁷ M, ≤10⁻⁸ M, ≤10⁻⁹ M, ≤10⁻¹⁰ M, ≤10⁻¹¹ M, or ≤10⁻¹² M. In some embodiments, a ligand specifically binds a receptor that is conserved among the receptors from different species. In some embodiments, specific binding can include, but does not require exclusive binding. Binding specificity of a ligand can be determined experimentally by methods known in the art. Such methods comprise, but are not limited to Western blots, ELISA-, RIA-, ECL-, IRMA-, EIA-, BIACORE™-tests and peptide scans.

“Amino acid” is used herein in its broadest sense, including both naturally occurring amino acids and non-naturally occurring amino acids, including amino acid analogs and derivatives. The latter includes molecules that contain amino acid moieties. Those skilled in the art will realize that according to this broad definition, amino acids herein include, for example, naturally occurring L-amino acids that form proteins; D-amino acids; chemically modified amino acids, such as amino acid analogs and derivatives; naturally occurring amino acids that do not form protein, such as norleucine, β-alanine, ornithine, GABA, etc.; and chemically synthesized compounds with amino acid characteristics known in the art. The term “protein-forming” as used herein refers to amino acids that can be incorporated into peptides, polypeptides, or proteins of cells through metabolic pathways.

As used herein, the term “substrate” when used in reference to a protease (e.g., metalloproteinase) is intended to mean any material or substance on which the protease (e.g., metalloproteinase) acts. The material or substance can be, for example, a naturally or non-naturally occurring organic chemical, or a macromolecule such as a polypeptide or peptidomimetic. In some embodiments, a metalloproteinase substrate specifically interacts with one or more metalloproteinases, and is cleaved by the metalloproteinase. At least one molecule of the substrate is cleaved by the metalloproteinase using appropriate conditions within the time frame of an experiment. In some embodiments, 10%, 20%, 30%, 40%, 50%, 60%, 70%, 80%, 90%, or 100% of the substrate can be cleaved by the metalloproteinase.

The term “functional analog” refers to a molecule that has the same biological specificity (e.g., binding to the same ligand) and/or activity (e.g., activating or inhibiting a target cell) as a reference molecule.

The term “prodrug” refers to a therapeutic molecule that is not active until activated in vivo.

The term “modulate” includes “increase”, “enhance” or “stimulate” as well as “decrease” or “reduce”, typically in a statistically or physiologically significant amount or degree relative to a control.

The term “variant” comprises one or more substitutions, additions, deletions and/or insertions relative to a reference polypeptide or polynucleotide. A variant of a polypeptide or polynucleotide comprises an amino acid or nucleotide sequence having at least about 50%, 55%, 60%, 65%, 70%, 75%, 80%, 85%, 90%, 91%, 92%, 93%, 94%, 95%, 96%, 97%, 98%, 99% or more sequence identity or similarity or homology to a reference sequence, as described herein, and substantially retains the activity of the reference sequence. Also included are sequences that consist of or differ from a reference sequence by the addition, deletion, insertion or substitution of 1, 2, 3, 4, 5, 6, 7, 8, 9, 10, 11, 12, 13, 14, 15, 16, 17, 18, 19, 20, 30, 40, 50, 60, 70, 80, 90, 100, 110, 120, 130, 140, 150 or more amino acids or nucleotides and that substantially retain at least one activity of the reference sequence. In certain embodiments, addition or deletion includes C-terminal and/or N-terminal addition and/or deletion.

The term “wild-type” refers to a gene or gene product (e.g., a polypeptide) that is most often observed in a population, and is thus set as the “normal” or “wild-type” form of the gene.

The term “linked” included covalently linked or non-covalently linked, referring to a first moiety, e.g., a first amino acid sequence or nucleotide sequence, covalently or non-covalently joined to a second moiety, e.g., a second amino acid sequence or nucleotide sequence, respectively. The first moiety can be directly joined or juxtaposed to the second moiety (referred to as directly linked, e.g., through peptide bond in the case of polypeptides) or, alternatively, intervening moiety (e.g., peptide linker) can be used to join the first moiety to the second moiety (referred to as indirectly linked), which can be said that the first moiety is linked to the second moiety through intervening moiety. In the case of polypeptides or proteins, the term “linked” not only includes a linkage of a first moiety (or a second moiety) at the C-terminus and/or the N-terminus, but also includes the linkage of the whole first moiety (or the second moiety) to any positions (e.g., amino acid residues not located in the terminal) of the second moiety (or the first moiety, respectively). In one aspect, the first moiety is linked to a second moiety by a peptide bond or a linker. In some embodiments, the first moiety can be linked to a second moiety by a phosphodiester bond or a linker. In some embodiments, the term “linker” is recognized as and refers to a molecule (including but not limited to unmodified or modified nucleic acids or amino acids) or group of molecules (for example, 2 or more, e.g., 2, 3, 4, 10, 30, 50, 100 or more) or any chemical moiety connecting two moieties, such as two polypeptides.

“Covalent bond” as used herein refers to a stable bond between two atoms sharing one or more electrons. Examples of covalent bonds include, but are not limited to, peptide bonds and disulfide bonds. As used herein, “peptide bond” refers to a covalent bond formed between a carboxyl group of an amino acid and an amine group of an adjacent amino acid. A “disulfide bond” as used herein refers to a covalent bond formed between two sulfur atoms, such as a combination of two Fc fragments by one or more disulfide bonds. One or more disulfide bonds may be formed between the two fragments by linking the thiol groups in the two fragments. In some embodiments, one or more disulfide bonds can be formed between one or more cysteines of two Fc fragments. Disulfide bonds can be formed by oxidation of two thiol groups. In some embodiments, the covalent linkage is directly linked by a covalent bond. In some embodiments, the covalent linkage is directly linked by a peptide bond or a disulfide bond.

The term “fused” or “fusion” in reference to two polypeptide sequences refers to the joining of the two polypeptide sequences through a backbone peptide bond. Two polypeptides may be fused directly or through a peptide linker that comprises one or more amino acids. Fusion proteins are polypeptides that comprise two or more regions derived from different or heterologous, proteins or peptides. Fusion proteins are prepared using conventional techniques of enzyme cutting and ligation of fragments from, desired sequences. PCR techniques employing synthetic oligonucleotides may be used to prepare and/or amplify the desired fragments. Overlapping synthetic oligonucleotide representing the desired sequences can also be used to prepare DNA constructs encoding fusion proteins. Fusion proteins can comprise several sequences, including a leader (or signal peptide) sequence, linker sequence, a leucine zipper sequence, or other oligomer-forming sequences, and sequences encoding highly antigenic moieties that provide a means for facile purification or rapid detection of a fusion protein. A fusion protein may be made by recombinant technology from a coding sequence containing the respective coding sequences for the two fusion partners, with or without a coding sequence for a peptide linker in between. In some embodiments, fusion encompasses chemical conjugation.

Half maximal inhibitory concentration (IC₅₀) is a measure of the effectiveness of a substance (e.g., ligand) in inhibiting a specific biological or biochemical function. It indicates how much of a particular drug or other substance (inhibitor, e.g., ligand) is needed to inhibit a given biological process by half. The values are typically expressed as molar concentration. IC₅₀ is comparable to an “EC₅₀” for agonist drug or other substance (e.g., ligand). EC₅₀ also represents the plasma concentration required for obtaining 50% of a maximum effect in vivo.

As used herein, an “IC₅₀” is used to indicate the effective concentration of a ligand needed to neutralize 50% of the receptor bioactivity in vitro. IC₅₀ or EC₅₀ can be measured by bioassays such as inhibition of ligand binding by FACS analysis (competition binding assay), cell-based cytokine release assay, or amplified luminescent proximity homogeneous assay (AlphaLISA)

“Percent (%) amino acid sequence identity” and “homology” with respect to a peptide or polypeptide sequence are defined as the percentage of amino acid residues in a candidate sequence that is identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MEGALIGN™ (DNASTAR) software. Those skilled in the art can determine appropriate parameters for measuring alignment, including any algorithms needed to achieve maximal alignment over the full length of the sequences being compared.

As used herein, the “C-terminus” of a polypeptide refers to the last amino acid residue of the polypeptide which donates its amine group to form a peptide bond with the carboxyl group of its adjacent amino acid residue. “N-terminus” of a polypeptide as used herein refers to the first amino acid of the polypeptide which donates its carboxyl group to form a peptide bond with the amine group of its adjacent amino acid residue.

As used herein, the term “moiety” refers to a portion of a molecule that has a distinct function within that molecule, and that function may be performed by that moiety in the context of another molecule. A moiety may be a chemical entity with a particular function or a portion of a biological molecule with a particular function.

As used herein, the terms “polypeptide”, “peptide” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified, for example, by disulfide bond formation, glycosylation, lipidation, acetylation, phosphorylation, or any other manipulation, such as conjugation with a labeling component. As used herein the term “amino acid” refers to either natural and/or unnatural or synthetic amino acids, including but not limited to glycine and both the D or L optical isomers, and amino acid analogs and peptidomimetics. Standard single or three letter codes are used to designate amino acids.

An “isolated” polypeptide is one that has been identified, separated and/or recovered from a component of its production environment (e.g., natural or recombinant). Preferably, the isolated polypeptide is free of association with all other components from its production environment. Contaminant components of its production environment, such as that resulting from recombinant transfected cells, are materials that would typically interfere with research, diagnostic or therapeutic uses for the polypeptide, and may include enzymes, hormones, and other proteinaceous or non-proteinaceous solutes. In some embodiments, the polypeptide will be purified: (1) to greater than 95% by weight of polypeptides as determined by, for example, the Lowry method, and in some embodiments, to greater than 99% by weight; (2) to a degree sufficient to obtain at least 15 residues of N-terminal or internal amino acid sequence by use of a spinning cup sequenator; or (3) to homogeneity by SDS-PAGE under non-reducing or reducing conditions using Coomassie Blue or, preferably, silver stain. Isolated polypeptide includes the polypeptide in situ within recombinant cells since at least one component of the polypeptide's natural environment will not be present. Ordinarily, however, an isolated polypeptide will be prepared by at least one purification step.

As used herein, the terms “polynucleotides”, “nucleic acids”, “nucleotides” and “oligonucleotides” are used interchangeably. They refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component.

An “isolated” nucleic acid molecule encoding a construct (such as the masking polypeptide described herein) is a nucleic acid molecule that is identified and separated from at least one contaminant nucleic acid molecule with which it is ordinarily associated in the environment in which it was produced. Preferably, the isolated nucleic acid is free of association with all components associated with the production environment. The isolated nucleic acid molecules encoding the polypeptides described herein are in a form other than in the form or setting in which it is found in nature. Isolated nucleic acid molecules therefore are distinguished from nucleic acid encoding the polypeptides described herein existing naturally in cells. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

The term “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

The term “transfected” or “transformed” or “transduced” as used herein refers to a process by which exogenous nucleic acid is transferred or introduced into the host cell. A “transfected” or “transformed” or “transduced” cell is one that has been transfected, transformed, or transduced with exogenous nucleic acid. The cell includes the primary subject cell and its progeny.

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that has the same function or biological activity as screened or selected for in the originally transformed cell are included herein.

The term “pharmaceutical formulation” or “pharmaceutical composition” refers to a preparation that is in such form as to permit the biological activity of the active ingredient to be effective, and that contains no additional components that are unacceptably toxic to a subject to which the formulation would be administered. Such formulations are sterile. A “sterile” formulation is aseptic or free from all living microorganisms and their spores.

It is understood that embodiments of the application described herein include “consisting of” and/or “consisting essentially of” embodiments.

Reference to “about” a value or parameter herein includes (and describes) variations that are directed to that value or parameter per se. For example, description referring to “about X” includes description of “X”.

As used herein, reference to “not” a value or parameter generally means and describes “other than” a value or parameter. For example, the method is not used to treat disease of type X means the method is used to treat disease of types other than X.

The term “about X-Y” used herein has the same meaning as “about X to about Y.”

As used herein and in the appended claims, the singular forms “a,” “or,” and “the” include plural referents unless the context clearly dictates otherwise.

### IL-15Rα or a Functional Fragment (S)

The IL-15Rα or a functional fragment thereof according to the present application can be any species of IL-15Rα or a functional fragment thereof.

In some embodiments, the IL-15Rα or a functional fragment thereof is selected from an extracellular region of human IL-15Rα or a sushi domain or functional analogs. In some embodiments, the IL-15Rα or a functional fragment is a C-terminal truncated form of the extracellular domain of IL-15Rα having the activity of IL-15Rα.

Extracellular region of IL-15Rα: The extracellular region of IL-15Rα is usually defined as the region of an IL-15Rα sequence that extends from its first N-terminal amino acid, to the last amino acid of the tail region (or region rich in glycosylation sites). The tail region of an IL-15Rα sequence can be determined by the skilled person, e.g., through the help of software.

IL-15Rα_sushi domain: The extracellular region of 1L-15Rα contains a domain, which is known as the sushi domain (Wei et al. 2001, J. Immunol. 167:277-282). The IL-15Rα_sushi domain has a beta sheet conformation.

The IL-15Rα sushi domain bears most of the binding affinity for IL-15, and behaves as a potent IL-15 agonist by enhancing its binding and biological effects (proliferation and protection from apoptosis) through the IL-15Rβγ heterodimer, whereas it does not affect IL-15 binding and function (Mortier E, et al. J Biol Chem. 2006 Jan. 20; 281(3):1612-9).

It is coded by exon 2 of IL-15Rα (Anderson D M, et al. Functional characterization of the human interleukin-15 receptor alpha chain and close linkage of IL15RA and IL2RA genes. J Biol Chem. 1995 Dec. 15; 270(50):29862-9). It begins at the first exon 2 encoded cysteine residue (C1), and ends at the fourth exon 2 encoded cysteine residue (C4). When considering the IL-15Rα protein sequence in the standard N-terminal to C-terminal orientation, the sushi domain of IL-15Rα can be defined as beginning at the first cysteine residue (C1) after the signal peptide, and ending at the fourth cysteine residue (C4) after the signal peptide. Residues C1 and C4 are both included in the sushi sequence. The IL-15Rα sushi domain can also be determined by analysis of the amino-acid sequence of IL-15Rα with appropriate software such as: Prosite (http://us.expasy.org/prosite/), (http://www.ebi.ac.uk/lnterProScan/), SMART (http://elm.eu.org/).

In some embodiments, the IL-15Rα_sushi domain comprises the amino acid sequence of SEQ ID NO: 9 or a variant thereof having at least about 80% (such as at least about any one of 80%, 85%, 90%, 95%, 96%, 97%, 98%, or 99%) sequence identity to the SEQ ID NO: 9.

In some embodiments, the IL-15Rα_sushi domain comprises the amino acid sequence of SEQ ID NO: 10 or a variant thereof having at least about 80% (such as at least about any one of 80%, 85%, 90%, 95%, 96%, 97%, 98%, or 99%) sequence identity to the SEQ ID NO: 10.

In some embodiments, the IL-15Rα_sushi domain comprises the amino acid sequence of SEQ ID NO: 11 or a variant thereof having at least about 80% (such as at least about any one of 80%, 85%, 90%, 95%, 96%, 97%, 98%, or 99%) sequence identity to the SEQ ID NO: 11.

### IL-15 Cytokine (I)

IL-15 is a member of the four α-helix bundle family with 14-15 kDa molecular weight and 114 amino acids (Fehniger T A, Caligiuri M A. Interleukin 15: biology and relevance to human disease. Blood. 2001; 97:14-32), and is produced by mononuclear phagocytes and other cells of the immune system. IL-15 is essential for natural killer cells (NK), natural killer T cells (NKT), and memory CD8+ T cells development and function.

IL-15 is a cytokine which like IL-2, has originally been described as a T cell growth factor. Both cytokines exert their cell signaling function through binding to a trimeric complex consisting of two shared receptors, the common gamma chain (yc; CD132) and IL-2 receptor beta-chain (IL-2Rα; CD122), as well as an alpha chain receptor unique to each cytokine: IL-2 receptor alpha (IL-2Rα; CD25) or IL-15 receptor alpha (IL-15Rα; CD215).

IL-15 shares components of the receptor for IL-2, the alpha chain of the IL-2 receptor (IL-2R) is not required, but both beta and common gamma chains are needed for IL-15 mediated bioactivities. (Giri J G, et al. IL-15, a novel T cell growth factor that shares activities and receptor components with IL-2. J Leukoc Biol. 1995 May; 57(5):763-6.). IL-15R consists of three subunits IL-15Rα, IL-2/IL-15Rβ, and γ chain, IL-15Rα is required for high-affinity binding but not signaling by IL-15. IL-15 functions mainly via trans-presentation (TP), during which an APC expressing IL-15 bound to IL-15Rα presents the ligand to the βγ receptor-heterodimer on a neighboring T/NK cell (Kenesei Á, Volkó J, et al. IL-15 Trans-Presentation Is an Autonomous, Antigen-Independent Process. J Immunol. 2021 Nov. 15; 207(10):2489-2500).

In some embodiments, the IL-15 cytokine also included IL-15 variants or functional fragments thereof. In eukaryotic cells, IL-15 is synthesized as a precursor polypeptide of 162 amino acids, which is then processed into mature IL-15 by the removal of amino acid residues 1-48. This results in a mature form of IL-15 consisting of 114 amino acids (amino acid residues 49-162) that are secreted in a mature, active form.

In some embodiments, the IL-15 cytokine comprises the amino acid sequence SEQ ID NO: 7 or a variant thereof having at least 90% (such as at least about any one of 91%, 92%, 93%, 94%, 95%, 96%, 97%, 98%, or 99%) sequence identity to the amino acid sequence of SEQ ID NO: 7.

In some embodiments, the IL-15 cytokine comprises the amino acid sequence SEQ ID NO: 8 or a variant thereof having at least 90% (such as at least about any one of 91%, 92%, 93%, 94%, 95%, 96%, 97%, 98%, or 99%) sequence identity to the amino acid sequence of SEQ ID NO: 8.

In some embodiments, the IL-15 cytokine comprises IL-15 variant or functional fragment thereof. In some embodiments, the IL-15 cytokine is any naturally occurring interleukin-15 (IL-15) protein. In some embodiments, the IL-15 cytokine is a modified variant thereof capable of binding to, or otherwise exhibiting affinity for, an interleukin-15 receptor (IL-15R) or component thereof (e.g., the IL-15Rα, IL-2/IL-15Rβ, and/or γ chain).

In some embodiments, the IL-15 cytokine comprises an amino acid sequence produced by at least one amino acid modification to the amino acid sequence of SEQ ID NO: 7. Each at least one amino acid modification can be any amino acid modification, such as a substitution, insertion, or deletion. Any combination of deletion, insertion, and substitution can be made to arrive at the final construct, provided that the final construct possesses the desired characteristics, e.g., retained/improved ligand-receptor binding, retained/enhanced bioactivity, etc. In some embodiments, the IL-15 cytokine comprises an amino acid sequence produced by at least 1, at least 2, at least 3, at least 4, at least 5, at least 6, at least 7, at least 8, at least 9, or at least 10 amino acid substitutions in the amino acid sequence of SEQ ID NO: 7.

### Linker

The term “linker” is recognized as and refers to a molecule (including but not limited to unmodified or modified nucleic acids or amino acids) or group of molecules (for example, 2 or more, e.g., 2, 3, 4, 10, 30, 50, 100 or more) or any chemical moiety connecting two moieties, such as two polypeptides.

In some embodiments, the linker is a peptide linker. In some embodiments, the peptide linker can have a naturally occurring sequence or a non-naturally occurring sequence. For example, a sequence derived from the hinge region of a heavy chain only antibody can be used as a linker. See, for example, WO1996/34103.

In some embodiments, the peptide linker is a human IgG1, IgG2, IgG3, or IgG4 hinge. In some embodiments, the peptide linker is a mutated human IgG1, IgG2, IgG3, or IgG4 hinge.

In some embodiments, the peptide linker is a flexible linker. Exemplary flexible linkers include, but are not limited to glycine polymers and glycine-serine polymers. In some embodiments, the non-cleavable linker is rich in amino acid residues G and S. In some embodiments, the non-cleavable linker includes a “G4S” repeat. In some embodiments, the non-cleavable linker is a polypeptide chain comprising at least 3 residues. Portions of such linkers may be flexible, hydrophilic, and have little or no secondary structure of their own (linker portions or flexible linker portions). Linkers of at least 3 amino acids may be used to join domains and/or regions that are positioned near to one another after the molecule has assembled. Longer linkers may also be used. In some embodiments, linkers may be about any one of: 1, 2, 3, 4, 5, 6, 7, 8, 9, 10, 11, 12, 13, 14, 15, 16, 17, 18, 19, 20, 25, 30, 35, 40, 45, 50, 100, 125, 150, 175 or 200 residues. When multiple linkers are used to interconnect portions of the molecule, the linkers may be the same or different (e.g., the same or different length and/or amino acid sequence).

In some embodiments, the peptide linker comprises or consists of a Gly-Ser linker. As used herein, the term “Gly-Ser linker” refers to a peptide that consists of glycine and serine residues. In some embodiments, an exemplary Gly-Ser linker comprises amino acid sequence of GSG (SEQ ID NO: 1). In some embodiments, an exemplary Gly-Ser linker comprises an amino acid sequence of the formula (Gly₄Ser)ₙ, wherein n is a positive integer (e.g., 1, 2, 3, 4, 5, 6, 7, 8, 9, or 10). In some embodiments, a preferred Gly-Ser linker is (Gly₄Ser)₁. In some embodiments, a preferred Gly-Ser linker is (Gly₄Ser)₂. In some embodiments, a preferred Gly-Ser linker is (Gly₄Ser)₃. In some embodiments, a preferred Gly-Ser linker is (Gly₄Ser)₄. In some embodiments, a preferred Gly-Ser linker is (Gly₄Ser)₅. In yet other aspects, two or more Gly-Ser linkers are incorporated in series in a polypeptide linker.

In some embodiments, the peptide linker comprises the amino acid sequence of any one of SEQ ID NOs: 1-5 or a variant thereof having at least 80% (such as at least about any one of 80%, 85%, 90%, 95%, 96%, 97%, 98%, or 99%) sequence identity to the amino acid sequence of any one of SEQ ID NOs: 1-5.

In some embodiments, the linkers can be classified as cleavable or non-cleavable. The cleavable moiety and non-cleavable linker are described below in IL-15 prodrugs part.

### The Linkage Between IL-15Rα or a Functional Fragment and Fc

In some embodiments, the IL-15Rα or a functional fragment and Fc domain is linked by a peptide linker.

In some embodiments, the peptide linker between IL-15Rα or a functional fragment and Fc comprises 3 to 40 amino acid residues.

In some embodiments, the peptide linker between IL-15Rα or a functional fragment and Fc comprises 3 to 25 amino acids residues.

In some embodiments, the peptide linker between IL-15Rα or a functional fragment and Fc comprises 20 to 25 amino acid residues.

In some embodiments, the peptide linker between IL-15Rα or a functional fragment and Fc comprises 25 amino acids residues.

In some embodiments, the peptide linker between IL-15Rα or a functional fragment and Fc is rich in G and S amino acid residues. In some preferred embodiments, the peptide linker consists of G and S amino acid residues.

In some embodiments, the peptide linker between IL-15Rα or a functional fragment and Fc is a flexible linker. Exemplary flexible linkers include, but are not limited to glycine polymers and glycine-serine polymers. In some embodiments, the peptide linker includes a “G4S” repeat. In some embodiments, the peptide linker is a polypeptide chain comprising at least 3 residues. Portions of such linkers may be flexible, hydrophilic, and have little or no secondary structure of their own (linker portions or flexible linker portions). In some embodiments, linkers of at least 3 amino acids may be used to join domains and/or regions that are positioned near to one another after the molecule has assembled. Longer peptide linker may also be used. In some embodiments, the peptide linkers may be about any one of 3, 4, 5, 6, 7, 8, 9, 10, 11, 12, 13, 14, 15, 16, 17, 18, 19, 20, 25, 30, 35 or 40 residues. When multiple linkers are used to interconnect portions of the molecule, the linkers may be the same or different (e.g., the same or different length and/or amino acid sequence).

In some embodiments, the peptide linker comprises or consists of a Gly-Ser linker. As used herein, the term “Gly-Ser linker” refers to a peptide that consists of glycine and serine residues. In some embodiments, an exemplary Gly-Ser linker comprises amino acid sequence of GSG (SEQ ID NO: 1). In some embodiments, an exemplary Gly-Ser linker comprises an amino acid sequence of the formula (Gly₄Ser)ₙ, wherein n is a positive integer (e.g., 1, 2, 3, 4, 5, 6, 7, 8, 9, or 10). In some embodiments, a preferred Gly-Ser linker is (Gly₄Ser)₁. In some embodiments, a preferred Gly-Ser linker is (Gly₄Ser)₂. In some embodiments, a preferred Gly-Ser linker is (Gly₄Ser)₃. In some embodiments, a preferred Gly-Ser linker is (Gly₄Ser)₄. In some embodiments, a preferred Gly-Ser linker is (Gly₄Ser)₅. In yet other aspects, two or more Gly-Ser linkers are incorporated in series in a polypeptide linker.

### The Linkage Between IL-15 and IL-15Rα or a Functional Fragment

In some embodiments, the IL-15 and IL-15Rα or a functional fragment can be linked through various methods, those skilled in the art are familiar with these methods.

In some embodiments, the IL-15 and IL-15Rα or a functional fragment is covalently or non-covalently linked.

In some embodiment, the IL-15 and IL-15Rα or a functional fragment is linked through a peptide bond.

In some embodiment, the IL-15 and IL-15Rα is linked through a peptide linker.

In some embodiments, the IL-15 and IL-15Rα or a functional fragment is non-covalently linked. IL-15 cytokine and IL-15Rα or a functional fragment can be non-covalently linked to form a “IL-15/IL-15Rα complex”.

### Half-Life Extension Moiety (C)

Preferably, the prodrug comprises an in vivo half-life extension moiety (C). The term half-life extension moiety refers to a moiety that extends the half-life of the target component in serum. A long half-life in vivo is important for therapeutic molecules, for example, cytokines that are administered to a subject generally have a short half-life since they are normally cleared rapidly from the subject by mechanisms including clearance by the kidney and endocytic degradation. Increasing the in vivo half-life of therapeutic molecules with naturally short half-lives allows for a more acceptable and manageable dosing regimen without sacrificing effectiveness. Thus, in the prodrug provided herein, preferably, a half-life extension moiety is linked to the biologically active moiety for the purpose of extending the half-life in vivo.

As used herein, a “half-life extension moiety” increases the in vivo half-life and improve PK, for example, by altering its size (e.g., to be above the kidney filtration cutoff), shape, hydrodynamic radius, charge, or parameters of absorption, biodistribution, metabolism, and elimination. An exemplary way to improve the PK of a polypeptide is by expression of an element in the polypeptide chain that binds to receptors that are recycled to the plasma membrane of cells rather than degraded in the lysosomes, such as the FcRn receptor on endothelial cells and transferrin receptor. Three types of proteins, e.g., human IgGs, HSA (or fragments), and transferrin, persist for much longer in human serum than would be predicted just by their size, which is a function of their ability to bind to receptors that are recycled rather than degraded in the lysosome. These proteins or fragments of them that retain the FcRn binding are routinely linked to other polypeptides to extend their serum half-life.

In some embodiments, the half-life extension moiety (C) can also be an antibody or antigen-binding fragment that binds to a protein with a long serum half-life such as serum albumin, transferrin, and the like. Examples of such antibodies or antigen-binding fragments thereof include a polyclonal antibody, a recombinant antibody, a human antibody, a humanized antibody a single-chain variable fragment (scFv), a single-domain antibody such as a heavy chain variable domain (V_{H}), a light chain variable domain (V_{L}) and a variable domain of camelid-type nanobody (V_{HH}), a dAb and the like.

In some embodiments, the half-life extension moiety (C) could also be functioned as a linker, optionally as a non-cleavable linker (L).

In some embodiments, the half-life extension moiety is an antibody Fc domain (e.g., a human IgG1, IgG2, IgG3, or IgG4 Fc) or fragment thereof that is capable of FcRn-mediated recycling, such as any heavy chain polypeptide or portion thereof that is capable of FcRn-mediated recycling. In some embodiments, the Fc domain is a monomer. In some embodiments, the Fc domain is a dimer, comprising a first Fc domain and a second Fc domain.

### Fc Domain

In some embodiments, the Fc domain is derived from any of IgA, IgD, IgE, IgG, and IgM, and subtypes thereof. IgG has the highest serum content and longest serum half-life among all immunoglobulins. Unlike other immunoglobulins, IgG is effectively recycled after binding to Fc receptors (FcRs). In some embodiments, the Fc domain is derived from an IgG (e.g., IgG1, IgG2, IgG3, or IgG4). In some embodiments, the Fc domain is derived from a human IgG. In some embodiments, the Fc domain comprises CH2 and CH3 domains. In some embodiments, the Fc domain further comprises full or part of the hinge region. In some embodiments, the Fc domain is derived from a human IgG1 or human IgG4. In some embodiments, the two subunits of the Fc domain dimerize via one or more (e.g., 1, 2, 3, 4, or more) disulfide bonds. In some embodiments, each subunit of the Fc domain comprises a full-length Fc sequence. In some embodiments, each subunit of the Fc domain comprises an N-terminus truncated Fc sequence, such as a truncated Fc domain with fewer N-terminal cysteines in order to reduce disulfide bond mispairing during dimerization. In some embodiments, the Fc domain is truncated at the N-terminus, e.g., lacks the first 1, 2, 3, 4, 5, 6, 7, 8, 9, or 10 amino acids of a complete immunoglobulin Fc domain.

In some embodiments, the Fc domain contains one or more mutations, such as insertion, deletion, and/or substitution.

In some embodiments, the Fc domain contains one or more amino acid mutations altering effector function, the Fc domain is engineered (e.g., comprises one or more amino acid mutations) to have altered binding to an FcR, specifically altered binding to an Fcγ receptor (responsible for ADCC), and/or altered effector function, such as altered antibody-dependent cell-mediated cytotoxicity (ADCC), Antibody-Dependent Cellular Phagocytosis (ADCP), and/or Complement-Dependent Cytotoxicity (CDC). Preferably, such amino acid mutation(s) does not reduce binding to FcRn receptors (responsible for half-life).

Fc domain (e.g., human IgG1 Fc) mutated to remove one or more effector functions such as ADCC, ADCP, or CDC, is hereinafter referred to as “effectless” or “almost effectless” Fc. For example, in some embodiments, the Fc is an effectless human IgG1 Fc comprising one or more of the following mutations (such as in each of Fc subunits): L234A, L235E, G237A, A330S, and P331S. In some embodiments, the Fc domain in the prodrug comprises L234A and L235A (“LALA”) mutations. The combinations of K322A, L234A, and L235A in IgG1 Fc are sufficient to almost completely abolish FcγR and C1q binding (Hezareh et al. J Virol 75, 12161-12168, 2001). MedImmune identified that a set of three mutations L234F/L235E/P331S have a very similar effect (Oganesyan et al., Acta Crystallographica 64, 700-704, 2008). In some embodiments, the Fc moiety comprises a modification of the glycosylation on N297 of the IgG1 Fc domain, which is known to be required for optimal FcR interaction. The Fc domain modification can be any suitable IgG Fc engineering mentioned in Wang et al. (“IgG Fc engineering to modulate antibody effector functions,” Protein Cell. 2018 January; 9(1): 63-73), the content of which is incorporated herein by reference in its entirety.

### Glycosylation Variants

In some embodiments, the Fc domain is altered to increase or decrease the extent to which the construct is glycosylated. The addition or deletion of glycosylation sites to an Fc domain may be conveniently accomplished by altering the amino acid sequence such that one or more glycosylation sites are created or removed.

Native Fc-containing proteins produced by mammalian cells typically comprise a branched, biantennary oligosaccharide that is generally attached by an N-linkage to Asn297 of the CH2 domain of the Fe region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an Fc moiety may be made in order to create certain improved properties.

In some embodiments, the Fc domain described herein is provided having a carbohydrate structure that lacks fucose attached (directly or indirectly) to the Fc domain. For example, the amount of fucose in such Fc domain may be from about 1% to about 80%, from about 1% to about 65%, from about 5% to about 65%, or from about 20% to about 40%. The amount of fucose is determined by calculating the average amount of fucose within the sugar chain at Asn297, relative to the sum of all glycostructures attached to Asn 297 (e.g., complex, hybrid and high mannose structures) as measured by MALDI-TOF mass spectrometry, as described in WO 2008/077546, for example. Asn297 refers to the asparagine residue located at about position 297 in the Fc domain (EU numbering of Fc region residues); however, Asn297 may also be located about ±3 amino acids upstream or downstream of position 297, i.e., between positions 294 and 300, due to minor sequence variations in Fc domains. Such fucosylation variants may have improved ADCC function. See, e.g., US Patent Publication Nos.

US 2003/0157108 (Presta, L.); US 2004/0093621 (Kyowa Hakko Kogyo Co., Ltd). Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al. J. Mol. Biol. 336:1239-1249 (2004); Yamane-Ohnuki et al. Biotech. Bioeng. 87: 614 (2004). Examples of cell lines capable of producing defucosylated Fc-containing proteins include Lec13 CHO cells deficient in protein fucosylation (Ripka et al. Arch. Biochem. Biophys. 249:533-545 (1986); US Patent Application No. US 2003/0157108 A1, Presta, L; and WO 2004/056312 A1, Adams et al., especially at Example 11), and knockout cell lines, such as alpha-1,6-fucosyltransferase gene, FUT8, knockout CHO cells (see, e.g., Yamane-Ohnuki et al. Biotech. Bioeng. 87: 614 (2004); Kanda, Y. et al., Biotechnol. Bioeng., 94(4):680-688 (2006); and WO2003/085107).

### Effector Function Variants

In some embodiments, the present application contemplates an Fc domain that possesses some but not all Fc effector functions, which makes it a desirable candidate for applications in which the half-life in vivo is important yet certain effector functions (such as CDC and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the Fc domain lacks FcγR binding (hence likely lacking ADCC activity), but retains FcRn binding ability. The primary cells for mediating ADCC, Natural Killer (NK) cells, express FcγRIII only, whereas monocytes express FcγRI, FcγRII and FcγRIII. FcR expression on hematopoietic cells is summarized in Table 2 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g., Hellstrom, I. et al. Proc. Nat'l Acad. Sci. USA 83:7059-7063 (1986)) and Hellstrom, I et al., Proc. Nat'l Acad. Sci. USA 82:1499-1502 (1985); 5,821,337 (see Bruggemann, M. et al., J. Exp. Med. 166:1351-1361 (1987)). Alternatively, non-radioactive assays methods may be employed (see, for example, ACTI™ non-radioactive cytotoxicity assay for flow cytometry (CellTechnology, Inc. Mountain View, CA; and CytoTox 96© non-radioactive cytotoxicity assay (Promega, Madison, WI). Useful effector cells for such assays include peripheral blood mononuclear cells (PBMC) and NK cells. Alternatively, or additionally, ADCC activity of the molecule of interest may be assessed in vivo, e.g., in an animal model such as that disclosed in Clynes et al. Proc. Nat'l Acad. Sci. USA 95:652-656 (1998). C1q binding assays may also be carried out to confirm that the Fc domain is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro et al., J. Immunol. Methods 202:163 (1996); Cragg, M. S. et al., Blood 101:1045-1052 (2003); and Cragg, M. S. and M. J. Glennie, Blood 103:2738-2743 (2004)). FcRn binding and in vivo clearance/half-life determinations can also be performed using methods known in the art (see, e.g., Petkova, S. B. et al., Int'l. Immunol. 18(12):1759-1769 (2006)).

Fc domain with reduced effector function includes those with substitution of one or more of Fc region residues 238, 265, 269, 270, 297, 327, and 329 (U.S. Pat. No. 6,737,056). Such Fc mutants include substitutions at two or more of amino acid positions 265, 269, 270, 297, and 327, including the so-called “DANA” Fc mutant with substitution of residues 265 and 297 to alanine (U.S. Pat. No. 7,332,581). Certain antibody variants with improved or diminished binding to FcRs are described (see, e.g., U.S. Pat. No. 6,737,056; WO 2004/056312, and Shields et al., J. Biol. Chem. 9(2): 6591-6604 (2001)). In some embodiments, alterations are made in the Fc domain that results in altered (i.e., either improved or diminished) C1q binding and/or CDC, e.g., as described in U.S. Pat. No. 6,194,551, WO 99/51642, and Idusogie et al. J. Immunol. 164: 4178-4184 (2000).

In some embodiments, the Fc domain comprises one or more amino acid substitutions, which increase the half-life and/or improve binding to the neonatal Fc receptor (FcRn).

Antibodies with increased half-lives and improved binding to the neonatal FcRn, which is responsible for the transfer of maternal IgGs to the fetus (Guyer et al., J. Immunol. 117:587 (1976) and Kim et al., J. Immunol. 24:249 (1994)), are described in US2005/0014934A1 (Hinton et al.). Those antibodies comprise an Fc domain with one or more substitutions therein which improve binding of the Fc region to FcRn. Such Fc variants include those with substitutions at one or more of Fc region residues, e.g., substitution of Fc region residue 434 (U.S. Pat. No. 7,371,826).

See also Duncan & Winter, Nature 322:738-40 (1988); U.S. Pat. Nos. 5,648,260; 5,624,821; and WO 94/29351 concerning other examples of Fc domain variants.

### Cysteine-Engineered Variants

In some embodiments, it may be desirable to create a cysteine-engineered Fc domain, in which one or more residues of an Fc domain are substituted with cysteine residues. In some embodiments, the substituted residues occur at accessible sites of the Fc domain. By substituting those residues with cysteine, reactive thiol groups are thereby positioned at accessible sites of the Fc domain and may be used to conjugate the molecule to other moieties, such as drug moieties or linker-drug moieties, to create a long-acting drug or prodrug conjugate.

In some embodiments, any one or more of the following residues may be substituted with cysteine: A1 18 (EU numbering) of the heavy chain; and S400 (EU numbering) of the heavy chain Fc domain. Cysteine engineered molecules may be generated as described, e.g., in U.S. Pat. No. 7,521,541.

In some embodiments, the Fc domain is derived from an IgG1 Fc. In some embodiments, the Fc domain is derived from a human IgG1 Fc. In some embodiments, the Fc moiety is a wildtype IgG1 Fc (IGHG1*05). In some embodiments, the Fc domain is a natural variant of IgG1 (e.g., IGHG1*03, which comprises D239E and L241M double mutations relative to IGHG1*05). In some embodiments, the Fc domain does not comprise the hinge region of an IgG1 Fc. In some embodiments, the Fc domain comprises at most about 5 amino acids truncated from the N-terminus of an IgG1 Fc, such as truncating the first, the first two, the first three, the first four, or the first five amino acids from the N-terminus of the IgG1 Fc. In some embodiments, the Fc domain comprises one or more ineffective mutations and/or deglycosylation mutation(s).

In some embodiments, the Fc domain is derived from an IgG4 Fe. In some embodiments, the Fc domain is derived from a human IgG4 Fe. In some embodiments, the Fc domain is a wildtype IgG4 Fe. In some embodiments, the Fc domain is a natural variant of IgG4. In some embodiments, the Fc domain does not comprise the hinge region of an IgG4 Fe. In some embodiments, the Fc domain comprises at most about 5 amino acids truncated from the N-terminus of an IgG4 Fe, such as truncating the first, the first two, the first three, the first four, or the first five amino acids from the N-terminus of the IgG4 Fe. In some embodiments, the Fc domain comprises one or more ineffective mutations and/or deglycosylation mutation(s).

Strategies of forming heterodimers of Fc-fusion polypeptides or bispecific antibodies are well known (see, e.g., Spies et al., Mol Inm. (2015) 67(2)(A): 95-106). For example, in some embodiments, the first and/or second polypeptide chain of Fc domain each contain one or more modifications that promote heterodimerization of the first and the second Fc domain.

As such, one or more amino acid modifications can be made to the first Fc domain and one or more amino acid modifications can be made to the second Fc domain using any strategy available in the art, including any strategy as described in Klein et al. (2012), MAbs, 4(6): 653-663. Exemplary strategies and modifications are the “knob into holes” approach. In some embodiments, the first Fc domain comprising a CH3 domain is a heavy chain polypeptide or a fragment thereof. The CH3 domains of the two Fe domains can be altered by the “knobs-into-holes” technology (Fe knob and Fe hole), which is described in detail with several examples in, e.g., WO 1996/027011; Ridgway, J. B. et al. Protein Eng (1996) 9(7): 617-621: Merchant, A. M., et al, Nat. Biotechnoi. (1998) 16(7): 677-681. See also Klein et al. (2012), MAbs, 4(6): 653-663. Using the knob-into-holes approach, the interaction surfaces of the two CH3 domains are altered to increase the heterodimerization of the two moieties containing the two altered CH-13 domains. This occurs by introducing a bulky residue into the CH3 domain of one of the Fe domains, which acts as the “knob.” Then, in order to accommodate the bulky residue, a “hole” is formed in the other Fc domain that can accommodate the knob. Either of the altered CH3 domains can be the “knob” while the other can be the “hole.” The introduction of a disulfide bridge further stabilizes the heterodimers (Merchant, A. M., et al, Nat. Biotechnoi (1998) 16(7); Atwell, S., et al, J. Mol, Biol. (1997) 270(1): 26-35) as well as increases yield. It is known that heterodimerization can be achieved by introducing the T366W and/or S354C mutations in a heavy chain to create the “knob” and by introducing the T366S, L368A, Y407V and/or Y349C mutations in a heavy chain to create the “hole” (numbering of the residues according to the Kabat EU numbering system). Carter et al. (2001), J. Immunol. Methods, 248: 7-15: Klein et al. (2012), MAbs, 4(6): 653-663.

In some embodiments, the Fc domain or fragment thereof includes the mutations T366S, L368A, and Y407V to form a ‘hole’. In some embodiments, the Fc domain or fragment thereof includes the mutation T366W to form a ‘knob’. In some embodiments, the Fc domain or fragment thereof includes the mutations Y349C, T366S, L368A, and Y407V to form a ‘hole’. In some embodiments, the Fc domain or fragment thereof includes the mutations S354C and T366W to form a ‘knob’. In some embodiments, the first Fc domain or fragment thereof includes the hole mutations, and the second Fc domain or fragment thereof includes the knob mutation. In some embodiments, the first Fc domain or fragment thereof includes the knob mutations, and the second Fc domain or fragment thereof includes the hole mutation, numbering of the residues according to the EU numbering system.

In some embodiments, the knobs-into-holes mutation are present in the Fe domains in addition to the LALA mutation.

In some embodiments, the first Fc domain comprises the amino acid sequence of SEQ ID NO: 12 or a variant thereof having at least 80% (such as at least about any one of 80%, 85%, 90%, 95%, 96%, 97%, 98%, or 99%) sequence identity to the amino acid sequence of SEQ ID NO: 12, and the second Fc domain comprises the amino acid sequence of SEQ ID NO: 13 or a variant thereof having at least 80% (such as at least about any one of 80%, 85%, 90%, 95%, 96%, 97%, 98%, or 99%) sequence identity to the amino acid sequence of SEQ ID NO: 13.

In some embodiments, the first Fc domain comprises the amino acid sequence of SEQ ID NO: 13 or a variant thereof having at least 80% (such as at least about any one of 80%, 85%, 90%, 95%, 96%, 97%, 98%, or 99%) sequence identity to the amino acid sequence of SEQ ID NO: 13, and the second Fc domain comprises the amino acid sequence of SEQ ID NO: 12 or a variant thereof having at least 80% (such as at least about any one of 80%, 85%, 90%, 95%, 96%, 97%, 98%, or 99%) sequence identity to the amino acid sequence of SEQ ID NO: 12.

### IL-15Rα or a Functional Fragment and Fc Fusion Protein:

In one aspect of the present application, there is provide a fusion protein comprising an Fc domain and an IL-15Rα or a functional fragment thereof (S).

In some embodiments, there is provided an IL-15Rα or a functional fragment and Fe fusion protein, wherein the IL-15Rα or a functional fragment thereof is linked to the Fc domain through a peptide linker. In some embodiments, the peptide linker is rich in G and S amino acid residues. In some embodiments, the peptide linker consists of G and S amino acid residues.

In some embodiments, there is provided an IL-15Rα or a functional fragment and Fc fusion protein, wherein the peptide linker connected the IL-15Rα or a functional fragment thereof and the Fc domain comprises 3 to 40 amino acid residues. In some embodiment, the peptide linker comprises 3 to 25 amino acids residues. In some embodiment, the peptide linker comprises 20 to 25 amino acids residues. In some embodiment, the peptide linker comprises 25 amino acids residues.

In some embodiments, there is provided an IL-15Rα or a functional fragment and Fc fusion protein, wherein the IL-15Rα or a functional fragment thereof is linked to the N-terminus or C-terminus of the Fc domain.

In some embodiments, there is provided an IL-15Rα or a functional fragment and Fc fusion protein, wherein the fusion protein further comprises IL-15 cytokine (I).

In some embodiments, there is provided an IL-15Rα or a functional fragment and Fc fusion protein, wherein the IL-15 cytokine is linked to the N-terminus or C-terminus of the Fc domain.

In some embodiments, there is provided an IL-15Rα or a functional fragment and Fc fusion protein, wherein the IL-15 is linked to the Fc domain through a covalent bond or a peptide linker.

In some embodiments, there is provided an IL-15Rα or a functional fragment and Fc fusion protein, wherein the IL-15Rα or a functional fragment is linked to the Fc domain, and the IL-15 cytokine is linked to the Fc domain.

In some embodiments, there is provided an IL-15Rα or a functional fragment and Fc fusion protein, wherein the IL-15Rα or a functional fragment is linked to the Fc domain, and the IL-15 cytokine is linked to t the IL-15Rα or a functional fragment.

In some embodiments, there is provided an IL-15Rα or a functional fragment and Fc fusion protein, wherein the fusion protein comprises the construct in an N to C-terminal or in a C to N-terminal direction: I-Fc-S, wherein the “-” represents covalent bond with or without peptide linker and wherein the peptide linker between S and Fc comprises 3 to 40 amino acid residues, preferably 3 to 25 amino acids residues, more preferably 20 to 25 amino acids residues, further more preferably 25 amino acids residues. Preferably, the peptide linker is rich in G and S amino acid residues; more preferably, the peptide linker consists of G and S amino acid residues.

In some embodiments, there is provided an IL-15Rα or a functional fragment and Fe fusion protein, wherein the fusion protein comprises the construct in an N to C-terminal or in a C to N- terminal direction: Fc-S-I, wherein the “-” represents covalent bond with or without peptide linker, and wherein the peptide linker between S and Fc comprises 3 to 40 amino acid residues, preferably 3 to 25 amino acids residues, more preferably 20 to 25 amino acids residues, further more preferably 25 amino acids residues. Preferably, the peptide linker is rich in G and S amino acid residues; more preferably, the peptide linker consists of G and S amino acid residues.

In some embodiment, there is provided an IL-15Rα or a functional fragment and Fc fusion protein, wherein the IL-15Rα or a functional fragment is linked to the Fc domain and co-expressed, while the IL-15 cytokine is transfected separately and a non-covalent IL-15/IL-15Rα complex can be formed.

In some embodiments, there is provided an IL-15Rα or a functional fragment and Fc fusion protein, wherein the IL-15Rα or a functional fragment is selected from an extracellular region of IL-15Rα or a sushi domain or functional analogs.

In some embodiments, there is provided an IL-15 and IL-15Rα dimer protein, wherein the dimer protein comprises any one of the IL-15Rα or a functional fragment and Fc fusion protein detailed described herein. In some embodiments, the dimer protein is monovalent. In some embodiments, the dimer protein is bivalent. In some embodiments, the dimer protein is a homodimer. In some embodiments, the dimer protein is a heterodimer.

### IL-15 and IL-15Rα Heterodimeric Protein

In some embodiments, there is provided an IL-15 and IL-15Rα heterodimeric protein comprising two monomers, wherein the heterodimeric protein comprises an IL-15Rα or a functional fragment, an IL-15 cytokine, and two Fc domain. In some embodiments, the peptide linker between Fc domain and IL-15Rα or a functional fragment comprises 3 to 40 amino acid residues, preferably 3 to 25 amino acids residues, more preferably 20 to 25 amino acids residues, further more preferably 25 amino acids residues. In some embodiments, the monomer is polypeptide.

In some embodiments, there is provided an IL-15 and IL-15Rα heterodimeric protein comprising two monomers, wherein the heterodimeric protein comprises an IL-15Rα or a functional fragment, an IL-15 cytokine, and two Fc domain, wherein in one monomer, the IL-15Rα or a functional fragment is linked to the first Fc domain through a peptide linker, and in the other monomer, the IL-15 cytokine is linked to the second Fc domain. In some embodiments, the peptide linker between Fc domain and IL-15Rα or a functional fragment comprises 3 to 40 amino acid residues, preferably 3 to 25 amino acids residues, more preferably 20 to 25 amino acids residues, further more preferably 25 amino acids residues. In some embodiments, the monomer is polypeptide.

In some embodiments, there is provided an IL-15 and IL-15Rα heterodimeric protein comprising two monomers, wherein the heterodimeric protein comprises an IL-15Rα or a functional fragment, an IL-15 cytokine, and two Fc domain, wherein in one monomer, the IL-15Rα or a functional fragment is linked to the first Fc domain through a peptide linker, and the IL-15 cytokine is also linked to the first Fc domain, and the other monomer comprises only the second Fc domain. In some embodiments, the peptide linker between Fc domain and IL-15Rα or a functional fragment comprises 3 to 40 amino acid residues, preferably 3 to 25 amino acids residues, more preferably 20 to 25 amino acids residues, further more preferably 25 amino acids residues. In some embodiments, the monomer is polypeptide.

In some embodiments, there is provided an IL-15 and IL-15Rα heterodimeric protein comprising two monomers, wherein the heterodimeric protein comprises an IL-15Rα or a functional fragment, an IL-15 cytokine, and two Fc domain, wherein in one monomer, the IL-15Rα or a functional fragment is linked to the first Fc domain through a peptide linker, and the IL-15 cytokine is linked to the IL-15Rα or a functional fragment, and the other monomer comprises only the second Fc domain. In some embodiments, the peptide linker between Fc domain and IL-15Rα or a functional fragment comprises 3 to 40 amino acid residues, preferably 3 to 25 amino acids residues, more preferably 20 to 25 amino acids residues, further more preferably 25 amino acids residues. In some embodiments, the IL-15 cytokine is covalently linked to the IL-15Rα or a functional fragment, optionally through a peptide linker. In some embodiments, the IL-15 cytokine is non-covalently linked to the IL-15Rα or a functional fragment and form an IL-15/IL-15Rα complex.

In some embodiments, the IL-15 and IL-15Rα heterodimeric protein described herein, wherein the peptide linker connected the IL-15Rα or a functional fragment thereof and the Fc domain comprises 3 to 40 amino acid residues. In some embodiments, the peptide linker comprises 3 to 25 amino acids residues. In some embodiments, the peptide linker comprises 20 to 25 amino acids residues. In some embodiment, the peptide linker comprises 25 amino acids residues. In some embodiments, the peptide linker is rich in G and S amino acid residues. In some embodiments, the peptide linker consists of G and S amino acid residues.

In some embodiments, the IL-15 and IL-15Rα heterodimeric protein described herein, wherein the IL-15Rα or a functional fragment thereof is linked to the N-terminus or C-terminus of the Fc domain.

In some embodiments, the IL-15 and IL-15Rα heterodimeric protein described herein, wherein the Fc domain is selected from the group consisting of a human IgG1 Fc domain, a human IgG2 Fc domain, a human IgG3 Fc domain, a human IgG4 Fc domain, an IgA Fc domain, an IgD Fc domain, an IgE Fc domain, and an IgM Fc domain; optionally, the Fc domain is a human IgG1 Fc domain. In some embodiments, the Fc domain is a human IgG1 Fc domain having L234A and L235A mutations, according to EU Numbering system. In some embodiments, the Fc domains comprises knobs-into-holes mutations (Fc knob and Fc hole). In some embodiments, the Fc knob is linked to the IL-15 cytokine, and the Fc hole is linked to the IL-15Rα or a functional fragment; or the Fc knob is linked to the IL-15Rα or a functional fragment, and the Fc hole is linked to the IL-15 cytokine. In some embodiments, the Fc knob comprises a T366W mutation in the first Fc domain, and the Fc hole comprises T366S, L368A, and Y407V mutations in the second Fc domain, according to EU Numbering system. In some embodiments, the Fc knob further comprises S354C mutation, and the Fc hole further comprises Y349C mutation, according to EU Numbering system.

In some embodiments, the IL-15 and IL-15Rα heterodimeric protein described herein, wherein the IL-15Rα or a functional fragment is selected from an extracellular region of IL-15Rα or a sushi domain or functional analogs.

In some embodiments, there is provided an IL-15 and IL-15Rα heterodimeric protein comprising two polypeptide chains, wherein one polypeptide chain one polypeptide chain comprises the amino acid sequence of SEQ ID NO: 16, or a variant thereof having at least about 80% (such as at least about any one of 80%, 85%, 90%, 95%, 96%, 97%, 98%, or 99%) sequence identity to the amino acid sequence of SEQ ID NO: 16; and the other polypeptide chain comprises the amino acid sequence of SEQ ID NO: 17, or a variant thereof having at least about 80% (such as at least about any one of 80%, 85%, 90%, 95%, 96%, 97%, 98%, or 99%) sequence identity to the amino acid sequence of SEQ ID NO: 17.

In some embodiments, there is provided an IL-15 and IL-15Rα heterodimeric protein comprising two polypeptide chains, wherein one polypeptide chain comprises the amino acid sequence of SEQ ID NO: 16, or a variant thereof having at least about 80% (such as at least about any one of 80%, 85%, 90%, 95%, 96%, 97%, 98%, or 99%) sequence identity to the amino acid sequence of SEQ ID NO: 16; and the other polypeptide chain comprises the amino acid sequence of SEQ ID NO: 18, or a variant thereof having at least about 80% (such as at least about any one of 80%, 85%, 90%, 95%, 96%, 97%, 98%, or 99%) sequence identity to the amino acid sequence of SEQ ID NO: 18.

### IL-15 Prodrugs

One way to reduce drug's side effects in therapeutic use includes providing a conditionally activatable prodrugs that the drug (e.g., IL-15) is linked to a masking moiety (MM) through cleavable moiety (CM). Masking moiety (e.g., a polypeptide making moiety, i.e., masking polypeptide) can act via steric hindrance to drug (e.g., IL-15). The cleavable moiety (CM) can be designed to be cleaved by proteases that are specific to certain tissues or pathologies, thus enabling the prodrug to be preferentially activated in desired locations (e.g., a tumor) to overcome the dosing amounts limitation of drug (e.g., IL-15).

In some embodiments, the IL-15 and IL-15Rα heterodimeric protein is in a form of prodrug (hereafter referred to as IL-15 prodrug), which further comprises cleavable moiety (CM) and masking moiety (e.g., masking polypeptide, MP), and wherein the masking moiety (MM) is linked to the heterodimeric protein through the cleavable moiety (CM). In the present application, the IL-15 prodrug described herein refers to the format of IL-15 and IL-15Rα heterodimeric protein comprising masking moiety (MM) and cleavable moiety (CM).

### Masking Moiety

A masking moiety as provided herein refers to a moiety capable of blocking the activity of the biologically active moiety. In some embodiments, the MM can be any moiety that inhibits the ability of the cytokine to bind and/or activate its receptor, e.g., a polypeptide (referred to as masking polypeptide, MP). In some embodiments, the MM can be any moiety that inhibits the ability of the antibody or antigen-binding fragment to bind to its target and/or, for example, inhibits cell proliferation, modulates cell activation and interactions, modulates the human immune system, or neutralizes antigens, e.g., a polypeptide (referred to as masking polypeptide, MP).

In some embodiments, the masking moiety can inhibit the ability of the cytokine to bind and/or activate its receptor sterically blocking and/or by noncovalently binding to the cytokine. Examples of suitable masking moieties include the full length or a cytokine-binding fragment or mutein of the cognate receptor of the cytokine. Antibodies and antigen-binding fragments thereof include a polyclonal antibody, a recombinant antibody, a human antibody, a humanized antibody a single-chain Fv (scFv), a single-domain antibody such as a heavy chain variable domain (V_{H}), a light chain variable domain (V_{L}) and a variable domain of camelid-type nanobody (V_{HH}), a dAb and the like that bind the cytokine can also be used. Further examples of suitable masking moieties include polypeptides that sterically inhibit or block the binding of the cytokine to its cognate receptor. For example, a peptide that is modified by conjugation to a water-soluble polymer, such as PEG, can sterically inhibit or prevent the binding of the cytokine to its receptor. Polypeptides, or fragments thereof, that have long serum half-lives can also be used, such as serum albumin (human serum albumin), immunoglobulin Fc, transferring, and the like, as well as fragments and muteins of such polypeptides. Antibodies and antigen-binding domains that bind to, for example, a protein with a long serum half-life such as HSA, immunoglobulin, or transferrin, or to a receptor that is recycled to the plasma membrane, such as FcRn or transferrin receptor, can also inhibit the cytokine, particularly when bound to their antigen. Other suitable antigen-binding domain that binds the cytokine can also be used, including non-immunoglobulin proteins that mimic antibody binding and/or structure such as anticalins, affilins, affibody molecules, affimers, affitins, alphabodies, avimers, DARPins, fynomers, Kunitz domain peptides, monobodies, and binding domains based on other engineered scaffolds such as SpA, GroEL, fibronectin, lipocalin, and CTLA4 scaffolds.

In some embodiments, the masking moiety is a synthetic masking polypeptide (MP), and the masking polypeptides (MP) have a larger hydrodynamic radius than their actual molecular weight. In some embodiments, the masking polypeptides only form a random coil, without a secondary structure. In some embodiments, the masking polypeptides have a steric masking effect that typically inhibits or blocks the activity of the biologically active moiety due to its proximity to the biologically active moiety and comparative size.

In some embodiments, the masking polypeptide (MP) comprises the amino acid sequence of SEQ ID NO: 14 or a variant thereof having at least about 90% (such as at least about any one of 91%, 92%, 93%, 94%, 95%, 96%, 97%, 98%, or 99%) sequence identity to the amino acid sequence of SEQ ID NO: 14.

In some embodiments, the masking polypeptide (MP) comprises the amino acid sequence of SEQ ID NO: 15 or a variant thereof having at least about 90% (such as at least about any one of 91%, 92%, 93%, 94%, 95%, 96%, 97%, 98%, or 99%) sequence identity to the amino acid sequence of SEQ ID NO: 15.

### Cleavable Moiety (CM)

The cleavable moiety (CM) is a polypeptide that comprises or is the cleavage site of an enzyme or a protease. In some embodiments, the proteases include but are not limited to urokinase-type plasminogen activator (uPA); matrix metalloproteinases (e.g., MMP1, MMP2, MMP3, MMP7, MMP8, MMP9, MMP10, MMP11, MMP12, MMP13, MMP14, MMP15, MMP16, MMP17, MMP19, MMP20, MMP23, MMP24, MMP26, and/or MMP27); Tobacco Etch Virus (TEV) protease; plasmin; Thrombin; PSA; PSMA; ADAMS/ADAMTS (e.g., ADAM8, ADAM9, ADAM10, ADAM12, ADAM13, ADAM17/TACE, ADAMDEC1, ADAMTS1, ADAMTS4, and/or ADAMTS5); caspases (e.g., Caspase-1, Caspase-2, Caspase-3, Caspase-4, Caspase-5, Caspase-6, Caspase-7, Caspase-8, Caspase-9, Caspase-10, Caspase-11, Caspase-12, Caspase-13, and/or Caspase-14); aspartate proteases (e.g., RACE and/or Renin); aspartic cathepsins (e.g., Cathepsin D and/or Cathepsin E); cysteine cathepsins (e.g., Cathepsin B, Cathepsin C, Cathepsin K, Cathepsin L, Cathepsin S, Cathepsin V/L2, and/or Cathepsin X/Z/P); cysteine proteinases (e.g., Cruzipain, Legumain, and/or Otubain-2); KLKs (e.g., KLK4, KLK5, KLK6, KLK7, KLK8, KLK10, KLK11, KLK13, and/or KLK14); metallo proteainases (e.g., Meprin, Neprilysin, PSMA, and/or BMP-1); serine proteases (e.g., activated protein C, Cathepsin A, Cathepsin G, Chymase, and/or coagulation factor proteases (such as FVIIa, FIXa, FXa, FXla, FXIIa)); elastase; granzyme B; guanidinobenzoatase; HtrAl; human neutrophil elastase; lactoferrin; marapsin; NS3/4A; PACE4; tPA; tryptase; type II transmembrane serine proteases (TTSPs) (e.g., DESC1, DPP-4, FAP, Hepsin, Matriptase-2, MT-SP1/Matriptase, TMPRSS2, TMPRSS3 and/or TMPRSS4); etc.

In some embodiments, the cleavable moiety (CM) comprises a substrate sequence for at least one matrix metalloprotease (MMP). Examples of MMPs include MMP1; MMP2; MMP3; MMP7; MMP8; MMP9; MMP10; MMP11; MMP12; MMP13; MMP14; MMP15; MMP16; MMP17; MMP19; MMP20; MMP23; MMP24; MMP26; and MMP27. In some embodiments, the CM comprises a substrate sequence for MMP2, MMP9, MMP14, MMP1, MMP3, MMP13, MMP17, MMP11 and MMP19. In some embodiments, the CM comprises a substrate sequence for MMP2. In some embodiments, the CM comprises a substrate sequence for MMP9. In some embodiments, the CM comprises a substrate sequence for two or more MMPs. In some embodiments, the CM comprises a substrate sequence for at least MMP2 and MMP9. In some embodiments, the CM comprises two or more substrates for the same MMP.

In some embodiments, the CM comprises at least two or more MMP2 substrates. In some embodiments, the CM comprises at least two or more MMP9 substrates.

The specificity of a protease for cleavage of a peptide bond with particular amino acids in nearby positions is described in terminology based on that originally created by Schechter & Berger (1967, 1968) to describe the specificity of papain. According to this model, amino acid residues in a substrate undergoing cleavage are designated P1, P2, P3, P4 etc. in the N-terminal direction from the cleaved bond. Likewise, the residues in C-terminal direction are designated P1′, P2′, P3′, P4′. etc.

In some embodiments, the CM comprises the amino acid sequence of SEQ ID NO: 6, or a variant thereof having at least about 90% (such as at least about any one of 91%, 92%, 93%, 94%, 95%, 96%, 97%, 98%, or 99%) sequence identity to the amino acid sequence of SEQ ID NO: 6.

In some embodiments, the IL-15 and IL-15Rα heterodimeric protein without masking polypeptide or the IL-15 prodrug, wherein the IL-15 cytokine or the IL-15Rα or a functional fragment thereof (e.g., IL-15Rα_sushi domain) has one or more conservative amino acid substitutions.

Insertion of non-naturally occurring amino acids, including synthetic non-natural amino acids, substituted amino acids, or one or more D-amino acids, into the peptides (e.g., the IL-15 cytokine in the IL-15 cytokine prodrug as described herein) can have multiple benefits. D-amino acid-containing peptides and the like exhibit increased stability in vitro or in vivo compared to their counterparts containing L-amino acid. Therefore, when greater intracellular stability is desired, the construction of peptides, such as by incorporation of D-amino acids, is particularly useful. Particularly, D-peptide and the like are resistant to endogenous peptidase and protease activity, thereby improving the bioavailability of the molecule and extending the lifespan in vivo when needed. In addition, D-peptide and the like cannot be effectively processed for limited presentation by type II major histocompatibility complexes (MHC) to T helper cells, so less prone to induce humoral immune responses in the subject.

Conservative substitutions are shown in Table A. More substantial changes are provided in Table A under the heading of “exemplary substitutions,” and as further described below in reference to amino acid side chain classes. Amino acids may be grouped according to common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; and (6) aromatic: Trp, Tyr, Phe. Non-conservative substitutions will entail exchanging a member of one of these classes for another class. Amino acid substitutions may be introduced into the protein constructs and the products screened for a desired activity mentioned above.

TABLE A
Amino acid substitutions

| Original | Exemplary | Preferred | Original | Exemplary | Preferred |
|---|---|---|---|---|---|
| Residue | Substitutions | Substitutions | Residue | Substitutions | Substitutions |
| Ala (A) | Val; Leu; Ile | Val | Leu (L) | Norleucine; Ile; | Ile |
| | | | | Val; Met; Ala; Phe | |
| Arg (R) | Lys; Gln; Asn | Lys | Lys (K) | Arg; Gln; Asn | Arg |
| Asn (N) | Gln; His; Asp, | Gln | Met (M) | Leu; Phe; Ile | Leu |
| | Lys; Arg | | | | |
| Asp (D) | Glu; Asn | Glu | Phe (F) | Trp; Leu; Val; Ile; | Tyr |
| | | | | Ala; Tyr | |
| Cys (C) | Ser; Ala | Ser | Pro (P) | Ala | Ala |
| Gln (Q) | Asn; Glu | Asn | Ser (S) | Thr | Thr |
| Glu (E) | Asp; Gln | Asp | Thr (T) | Val; Ser | Ser |
| Gly (G) | Ala | Ala | Trp (W) | Tyr; Phe | Tyr |
| His (H) | Asn; Gln; Lys; | Arg | Tyr (Y) | Trp; Phe; Thr; Ser | Phe |
| | Arg | | | | |
| Ile (I) | Leu; Val; Met; | Leu | Val (V) | Ile; Leu; Met; Phe; | Leu |
| | Ala; Phe; | | | Ala; Norleucine | |
| | Norleucine | | | | |

In some embodiments, the IL-15 prodrug provided herein comprises the constructs showing in Table 1.

In some embodiments, the IL-15 prodrug provided herein comprises two polypeptide chains, wherein one polypeptide chain comprises the amino acid sequence of SEQ ID NO: 19 or a variant thereof having at least about 80% (such as at least about any one of 80%, 85%, 90%, 95%, 96%, 97%, 98%, or 99%) sequence identity to the amino acid sequence of SEQ ID NO: 19, and the other polypeptide chain comprises the amino acid sequence of SEQ ID NO: 20 or a variant thereof having at least about 80% (such as at least about any one of 80%, 85%, 90%, 95%, 96%, 97%, 98%, or 99%) sequence identity to the amino acid sequence SEQ ID NO: 20.

In some embodiments, the IL-15 prodrug provided herein comprises two polypeptide chains, wherein one polypeptide chain comprises the amino acid sequence of SEQ ID NO: 19 or a variant thereof having at least about 80% (such as at least about any one of 80%, 85%, 90%, 95%, 96%, 97%, 98%, or 99%) sequence identity to the amino acid sequence of SEQ ID NO: 19, and the other polypeptide chain comprises the amino acid sequence of SEQ ID NO: 18 or a variant thereof having at least about 80% (such as at least about any one of 80%, 85%, 90%, 95%, 96%, 97%, 98%, or 99%) sequence identity to the amino acid sequence SEQ ID NO: 18.

In some embodiments, the IL-15 prodrug provided herein comprises two polypeptide chains, wherein one polypeptide chain comprises the amino acid sequence of SEQ ID NO: 21 or a variant thereof having at least about 80% (such as at least about any one of 80%, 85%, 90%, 95%, 96%, 97%, 98%, or 99%) sequence identity to the amino acid sequence of SEQ ID NO: 21, and the other polypeptide chain comprises the amino acid sequence of SEQ ID NO: 22 or a variant thereof having at least about 80% (such as at least about any one of 80%, 85%, 90%, 95%, 96%, 97%, 98%, or 99%) sequence identity to the amino acid sequence SEQ ID NO: 22.

In some embodiments, the IL-15 prodrug provided herein comprises two polypeptide chains, wherein one polypeptide chain comprises the amino acid sequence of SEQ ID NO: 23 or a variant thereof having at least about 80% (such as at least about any one of 80%, 85%, 90%, 95%, 96%, 97%, 98%, or 99%) sequence identity to the amino acid sequence of SEQ ID NO: 23, and the other polypeptide chain comprises the amino acid sequence of SEQ ID NO: 22 or a variant thereof having at least about 80% (such as at least about any one of 80%, 85%, 90%, 95%, 96%, 97%, 98%, or 99%) sequence identity to the amino acid sequence SEQ ID NO: 22.

In some embodiments, the IL-15 prodrug provided herein comprises two polypeptide chains, wherein one polypeptide chain comprises the amino acid sequence of SEQ ID NO: 24 or a variant thereof having at least about 80% (such as at least about any one of 80%, 85%, 90%, 95%, 96%, 97%, 98%, or 99%) sequence identity to the amino acid sequence of SEQ ID NO: 24, and the other polypeptide chain comprises the amino acid sequence of SEQ ID NO: 22 or a variant thereof having at least about 80% (such as at least about any one of 80%, 85%, 90%, 95%, 96%, 97%, 98%, or 99%) sequence identity to the amino acid sequence SEQ ID NO: 22.

### Binding Affinity

Binding affinity of a molecule (e.g., IL-15 or functional fragment thereof) and its binding partner (e.g., IL-2/IL-15Rβγ) can be determined experimentally by any suitable ligand binding assays or antibody/antigen binding assays known in the art, e.g., Western blots, sandwich enzyme-linked immunosorbent assay (ELISA), Meso Scale Discovery (MSD) electrochemiluminescence, bead based multiplex immunoassays (MIA), RIA, Surface Plasma Resonance (SPR), ECL, IRMA, EIA, Biacore assay, Octet analysis, peptide scans, etc. For example, easy analysis is possible by using an IL-15 or functional fragment thereof, or its receptor (e.g., IL-2/IL-15Rβγ) or subunits thereof marked with a variety of marker agents, as well as by using BiacoreX (Amersham Biosciences), which is an over-the-counter, measuring kit, or similar kit, according to the user's manual and experiment operation method attached with the kit.

In some embodiments, protein microarray is used for analyzing the interaction, function, and activity of the IL-15 or functional fragment thereof, described herein to its receptor, on a large scale. The protein chip has a support surface-bound with a range of capture proteins (e.g., IL-15 receptor or subunits thereof). Fluorescently labeled probe molecules (e.g., IL-15 or functional fragment thereof described herein) are then added to the array and upon interaction with the bound capture protein, a fluorescent signal is released and read by a laser scanner.

Binding affinity can also be measured using SPR (Biacore T-200). For example, anti-human IgG antibody is coupled to the surface of a CM-5 sensor chip using EDC/NHS chemistry. Then human IL-2/IL-15Rβγ-Fc fusion protein is used as the captured ligand over this surface. Serial dilutions of IL-15 and IL-15Rα heterodimeric protein described herein are allowed to bind to the captured ligands, and the binding and dissociation of IL-15 and IL-15Rα heterodimeric protein to IL-2/IL-15Rβγ can be monitored in real time. Equilibrium dissociation constant (Kd) and dissociation rate constant can be determined by performing kinetic analysis using BIA evaluation software.

### Pharmacokinetics (PK)

Pharmacokinetics (PK) refers to the absorption, distribution, metabolism, and excretion of a drug (e.g., IL-15 and IL-15Rα heterodimeric protein described herein) once it has been administered to a subject. Pharmacokinetic parameters that may be useful in determining clinical utility include but are not limited to serum/plasma concentration, serum/plasma concentration over time, maximum serum/plasma concentration (Cₘₐₓ), time to reach maximum concentration (Tₘₐₓ), half-life (t_{1/2}), area under concentration time curve within the dosing interval (AUC_{τ}), etc.

Techniques for obtaining a PK curve of a drug, such as IL-15 and IL-15Rα heterodimeric protein described herein, are known in the art. See, e.g., Heller et al., Annu Rev Anal Chem, 11, 2018; and Ghandforoush-Sattari et al., J Amino Acids, Article ID 346237, Volume 2010. In some embodiments, the PK curves of the IL-15 and IL-15Rα heterodimeric protein described herein in the individual is measured in a blood, plasma, or serum sample from the individual. In some embodiments, the PK curves of the IL-15 and IL-15Rα heterodimeric protein described herein in the individual is measured using a mass spectrometry technique, such as LC-MS/MS, or ELISA. PK analysis on PK curves can be conducted by any methods known in the art, such as non-compartmental analysis, e.g., using PKSolver V2 software (Zhang Y. et al., “PKSolver: An add-in program for pharmacokinetic and pharmacodynamic data analysis in Microsoft Excel,” Comput Methods Programs Biomed. 2010; 99(3):306-1).

“C” denotes the concentration of drug (e.g., IL-15 and IL-15Rα heterodimeric protein) in blood plasma, serum, or in any appropriate body fluid or tissue of a subject, and is generally expressed as mass per unit volume, for example nanograms per milliliter. For convenience, the concentration of drug in serum or plasma is referred to herein as “serum concentration” or “plasma concentration.” The serum/plasma concentration at any time following drug administration (e.g., IL-15 and IL-15Rα heterodimeric protein, such as i.v., i.p., or s.c. administration) is referenced as Cₜᵢₘₑ or Cₜ. The maximum serum/plasma drug concentration during the dosing period is referenced as Cₘₐₓ, while Cₘᵢₙ refers to the minimum serum/plasma drug concentration at the end of a dosing interval; and Cₐᵥₑ refers to an average concentration during the dosing interval.

The term “bioavailability” refers to an extent to which—and sometimes rate at which—the drug (e.g., IL-15 and IL-15Rα heterodimeric protein) enters systemic circulation, thereby gaining access to the site of action.

“AUC” is the area under the serum/plasma concentration-time curve and is considered to be the most reliable measure of bioavailability, such as area under concentration time curve within the dosing interval (AUC_{τ}), “overall exposure” or “total drug exposure across time” (AUC₀₋ₗₐₛₜ or AUC_{0-inf}), area under concentration time curve at time t post-administration (AUC₀₋ₜ), etc.

Serum/plasma concentration peak time (Tₘₐₓ) is the time when peak serum/plasma concentration (Cₘₐₓ) is reached after administration of a drug (e.g., IL-15 and IL-15Rα heterodimeric protein).

Half-life (t_{1/2}) is the amount of time required for the drug concentration (e.g., IL-15 and IL-15Rα heterodimeric protein) measured in plasma or serum (or other biological matrices) to be reduced to exactly half of its concentration or amount at certain time point. For example, after iv dosing, the drug concentrations in plasma or serum decline due to both distribution and elimination. In a plasma or serum profile of drug concentration over time post- iv doing, the first phase or rapid decline is considered to be primarily due to distribution, while the later phase of decline is usually slower and considered to be primarily due to elimination, although both processes occur in both phases. Distribution is assumed to be complete after sufficient time. In general, the elimination half-life is determined from the terminal or elimination (dominant) phase of the plasma/serum concentration versus time curve. See, e.g., Michael Schrag and Kelly Regal, “Chapter 3—Pharmacokinetics and Toxicokinetics” of “A Comprehensive Guide to Toxicology in Preclinical Drug Development”, 2013.

### Stability

In some embodiments, the fusion protein or heterodimeric protein described herein (e.g., IL-15 and IL-15Rα heterodimeric protein) described herein have excellent stability, such as physical stability, chemical stability, and/or biological stability. In some embodiments, the IL-15 and IL-15Rα heterodimeric protein described herein have superior stability under accelerated stress (e.g., high temperature), such as less or no fragmentation, aggregate formation, and/or aggregate increment.

Stability of protein, in particular the susceptibility to aggregation, is primarily determined by the conformational and the colloidal stability of the protein molecules. It is generally believed that the first step in non-native protein aggregation, which is the most prevalent form of aggregation, is a slight perturbation of the molecular structure, e.g., a partial unfolding of the protein, i.e., a conformational change. This is determined by the conformational stability of the protein. In the second step, the partially unfolded molecules then come into close proximity, being driven by diffusion and random Brownian motion, to form aggregates. This second step is primarily governed by the colloidal stability of the molecules (see Chi et al., Roles of conformational stability and colloidal stability in the aggregation of recombinant human granulocyte colony stimulating factor. Protein Science, 2003 May; 12(5): 903-913). As used herein, the term “stability” generally is related to maintaining the integrity or to minimizing the degradation, denaturation, aggregation or unfolding of a biologically active agent such as a protein. As used herein, “improved stability” generally means that, under conditions known to result in degradation, denaturation, aggregation or unfolding, the protein (e.g., IL-15 and IL-15Rα heterodimeric protein described herein) of interest maintains greater stability compared to a control protein (e.g., other IL-15 and IL-15Rα heterodimeric protein).

Differential scanning calorimetry (DSC) and differential scanning fluorimetry (DSF) are well known techniques in the art that are used to predict the stability of a protein formulation. Specifically, these techniques can be used to determine the unfolding temperature (Tm) of a protein in given formulation. It is standard in the art to correlate high Tm measurements for a protein in given formulation with more robust and stable protein formulations for long-term, shelf-stable storage.

A “stable” heterodimeric protein or prodrug (or formulation), e.g., IL-15 and IL-15Rα heterodimeric protein described herein, essentially retains its physical stability and/or chemical stability and/or biological activity during the manufacturing process and/or upon storage.

Various analytical techniques for measuring protein stability are available in the art and are reviewed in Peptide and Protein Drug Delivery, 247-301, Vincent Lee Ed., Marcel Dekker, Inc., New York, N.Y., Pubs. (1991) and Jones, A. (1993) Adv. Drug Delivery Rev. 10: 29-90. For example, in one embodiment, the stability of the protein is determined according to the percentage of monomer protein in the solution, with a low percentage of degraded (e.g., fragmented) and/or aggregated protein. Preferably, the protein (or formulation) is stable at room temperature (about 30° C.) or at 40° C. for at least 1 month and/or stable at about 2-8° C. for at least 6 months, or for at least 1 year or for at least 2 years. Furthermore, the protein (or formulation) is preferably stable following freezing (to, e.g., −70° C.) and thawing, hereinafter referred to as a “freeze/thaw cycle.”

A heterodimeric protein, e.g., IL-15 and IL-15Rα heterodimeric protein described herein, “retains its physical stability” in a formulation if it shows substantially no signs of instability, e.g., aggregation, precipitation and/or denaturation, upon visual examination of color and/or clarity or as measured by UV light scattering or by size exclusion chromatography.

Aggregation is a process whereby individual protein molecules or complexes associate covalently or non-covalently to form aggregates. Aggregation can proceed to the extent that a visible precipitate is formed.

A heterodimeric protein, e.g., IL-15 and IL-15Rα heterodimeric protein described herein, “retains its chemical stability” in a formulation, if the chemical stability at a given time is such that the protein is considered to still retain its biological activity (e.g., as mentioned in “Bioactivity” subsection above). Chemical stability can be assessed by, e.g., detecting and quantifying chemically altered forms of the protein. Chemical alteration may involve size modification (e.g., clipping) which can be evaluated using size exclusion chromatography, SDS-PAGE and/or matrix-assisted laser desorption ionization/time-of-flight mass spectrometry (MALDI/TOF MS), for example. Other types of chemical alteration include charge alteration (e.g., occurring as a result of deamidation or oxidation) which can be evaluated by ion-exchange chromatography, for example.

A heterodimeric protein, e.g., IL-15 and IL-15Rα heterodimeric protein described herein, “retains its biological activity” in a formulation, if the protein, in a pharmaceutical formulation is biologically active for its intended purpose. For example, biological activity is retained if the biological activity of the protein, in the formulation is within about 30%, about 20%, or about 10% (within the errors of the assay) of the biological activity exhibited at the time the formulation was prepared.

One of skilled in the art will appreciate that stability of a heterodimeric protein (e.g., IL-15 and IL-15Rα heterodimeric protein described herein) is dependent on other features in addition to the composition of the formulation. For example, stability can be affected by temperature, pressure, humidity, pH, and external forms of radiation. Stability of a protein (e.g., IL-15 and IL-15Rα heterodimeric protein) in a protein formulation can be determined by various means. In some embodiments, the protein stability is determined by size exclusion chromatography (SEC). SEC separates analytes (e.g., macromolecules such as proteins) on the basis of a combination of their hydrodynamic size, diffusion coefficient, and surface properties. Thus, for example, SEC can separate IL-15 and IL-15Rα heterodimeric protein described herein in their natural three-dimensional conformation from proteins in various states of denaturation, and/or proteins that have been degraded. In SEC, the stationary phase is generally composed of inert particles packed into a dense three-dimensional matrix within a glass or steel column. The mobile phase can be pure water, an aqueous buffer, an organic solvent, mixtures of these, or other solvents. The stationary-phase particles have small pores and/or channels which will only allow species below a certain size to enter. Large particles are therefore excluded from these pores and channels, but the smaller particles are removed from the flowing mobile phase. The time particles spend immobilized in the stationary-phase pores depends, in part, on how far into the pores they can penetrate. Their removal from the mobile phase flow causes them to take longer to elute from the column and results in a separation between the particles based on differences in their size.

In some embodiments, SEC is combined with an identification technique to identify or characterize proteins (e.g., IL-15 and IL-15Rα heterodimeric protein), or fragments thereof. Protein identification and characterization can be accomplished by various techniques, including but not limited chromatographic techniques, e.g., high-performance liquid chromatography (HPLC), Capillary Electrophoresis-Sodium Dodecyl Sulfate (CE-SDS), immunoassays, electrophoresis, ultra-violet/visible/infrared spectroscopy, raman spectroscopy, surface enhanced raman spectroscopy, mass spectroscopy, gas chromatography, static light scattering (SLS), Fourier Transform Infrared Spectroscopy (FTIR), circular dichroism (CD), urea-induced protein unfolding techniques, intrinsic tryptophan fluorescence, differential scanning calorimetry, and/or ANS protein binding.

In some embodiments, sample formulations (e.g., comprising the IL-15 and IL-15Rα heterodimeric protein described herein) and reference formulations are optionally assayed prior to a treatment phase to determine the content of monomer, aggregated and/or fragmented protein (and/or fragmentation increase %, aggregation increase %, etc.). Subsequently, each of the protein formulations undergoes a treatment phase. For example, each protein formulation may be stored for an extended period (e.g., 3 months, 6 months, 12 months, or longer) at a specific temperature (e.g., 40° C., 25° C., or 5° C.). In some embodiments, the protein formulations undergo a physical stress test such as stir-stress assay. In some embodiments, the protein formulations undergo accelerated stability test, such as treated under accelerated stress, including high temperature (e.g., 40° C.), high humidity, and/or low pH, etc. In some embodiments, the protein formulations undergo cycles of freezing and thawing. In some embodiments, samples of the same protein formulation receive differential treatment, e.g., storage for a period of time in different temperatures. Following the treatment phase, the protein formulations are assayed to determine the content of protein monomer, aggregates and/or fragments (and/or fragmentation increase %, aggregation increase %, etc.).

“Substantial protein aggregation” refers to a level of protein aggregation in a protein formulation that is substantially greater than the level of protein aggregation in a reference protein formulation. The reference protein formulation may be the same protein formulation before a period of storage or before a treatment (e.g., before subjected to a destabilizing condition, such as elevated temperature, humidity, pH, and/or to long term storage.).

“Substantially free of protein aggregation” refers to proteins (or formulations) of the application that do not have a significantly greater level or percentage of aggregated protein than a reference formulation. In some embodiments, the stability is measured by SEC. In some embodiments, the stability is measured by CE-SDS.

In some embodiments, stability refers to reduced fragmentation of the IL-15 and IL-15Rα heterodimeric protein described herein. The term “low to undetectable levels of fragmentation” as used herein refers to samples containing equal to or more than 80%, 85%, 90%, 95%, 98% or 99% of the total protein, for example, in a single peak as determined by HPSEC, or in multiple peaks (e.g., as many peaks as there are subunits) by reduced Capillary Gel Electrophoresis (rCGE), representing the non-degraded protein or a non-degraded fragment thereof, and containing no other single peaks having more than 5%, more than 4%, more than 3%, more than 2%, more than 1%, or more than 0.5% of the total protein in each. The term “reduced Capillary Gel Electrophoresis” as used herein refers to capillary gel electrophoresis under reducing conditions sufficient to reduce disulfide bonds in an Fc-containing protein, such as the IL-15Rα and Fc fusion protein or IL-15 prodrug described herein.

### Vectors

The present application also provides isolated nucleic acids encoding any of the fusion protein, any of the heterodimeric protein or any of the prodrugs (e.g., IL-15 prodrug) described herein, vectors comprising the nucleic acids described herein. Also provided are isolated host cells (e.g., CHO cells, HEK 293 cells, Hela cells, or COS cells) comprising nucleic acids or vectors described herein. Suitable nucleic acid constructs include, but are not limited to, constructs that are capable of expression in prokaryotic or eukaryotic cells. Expression constructs are generally selected so as to be compatible with the host cell in which they are to be used. In some embodiments, the vector encodes a protein or prodrugs (e.g., IL-15 and IL-15Rα heterodimeric protein).

In some embodiments, the vector comprising a nucleic acid encoding the IL-15 and IL-15Rα heterodimeric protein, or any components of the protein or prodrugs described herein is suitable for replication and integration in eukaryotic cells, such as mammalian cells (e.g., CHO cells, HEK 293 cells, Hela cells, COS cells). In some embodiments, the vector is a viral vector. In some embodiments, the vector is a non-viral vector, such as pTT5.

A number of viral based systems have been developed for gene transfer into mammalian cells. Examples of viral vectors include, but are not limited to, adenoviral vectors, adeno-associated virus vectors, lentiviral vectors, retroviral vectors, herpes simplex viral vectors, and derivatives thereof. Viral vector technology is well known in the art and is described, for example, in Sambrook et al. (2001, Molecular Cloning: A Laboratory Manual, Cold Spring Harbor Laboratory, New York), and in other virology and molecular biology manuals. Retroviruses provide a convenient platform for gene delivery systems. The heterologous nucleic acid can be inserted into a vector and packaged in retroviral particles using techniques known in the art. The recombinant virus can then be isolated and delivered to the engineered mammalian cell in vitro or ex vivo. A number of retroviral systems are known in the art. In some embodiments, adenovirus vectors are used. A number of adenovirus vectors are known in the art. In some embodiments, lentivirus vectors are used. In some embodiments, self-inactivating lentiviral vectors are used. For example, self-inactivating lentiviral vectors carrying the construct protein coding sequence(s) can be packaged with protocols known in the art. The resulting lentiviral vectors can be used to transduce a mammalian cell using methods known in the art. Vectors derived from retroviruses such as lentivirus are suitable tools to achieve long-term gene transfer, because they allow long-term, stable integration of a transgene and its propagation in progeny cells. Lentiviral vectors also have low immunogenicity, and can transduce non-proliferating cells.

In some embodiments, the vector is a non-viral vector. In some embodiments, the vector is a pTT5 vector. In some embodiments, the vector is a transposon, such as a Sleeping Beauty (SB) transposon system, or a PiggyBac transposon system. In some embodiments, the vector is a polymer-based non-viral vector, including for example, poly (lactic-co-glycolic acid) (PLGA) and poly lactic acid (PLA), poly (ethylene imine) (PEI), and dendrimers. In some embodiments, the vector is a cationic-lipid based non-viral vector, such as cationic liposome, lipid nanoemulsion, and solid lipid nanoparticle (SLN). In some embodiments, the vector is a peptide-based gene non-viral vector, such as Poly-L-lysine. Any of the known non-viral vectors suitable for genome editing can be used for introducing the IL-15 prodrug-encoding nucleic acid(s) to the host cells. See, for example, Yin H. et al., Nature Rev. Genetics (2014) 15:521-555; Aronovich E L et al. “The Sleeping Beauty transposon system: a non-viral vector for gene therapy.” Hum. Mol. Genet. (2011) R1: R14-20; and Zhao S. et al. “PiggyBac transposon vectors: the tools of the human gene editing.” Transl. Lung Cancer Res. (2016) 5(1): 120-125, which are incorporated herein by reference. In some embodiments, any one or more of the nucleic acids or vectors encoding the prodrugs described herein is introduced to the host cells (e.g., CHO, HEK 293, Hela, or COS) by a physical method, including, but not limited to electroporation, sonoporation, photoporation, magnetofection, hydroporation.

In some embodiments, the vector contains a selectable marker gene or a reporter gene to select cells expressing the prodrugs described herein from the population of host cells transfected through vectors (e.g., lentiviral vectors, pTT5 vectors). Both selectable markers and reporter genes may be flanked by appropriate regulatory sequences to enable expression in the host cells. For example, the vector may contain transcription and translation terminators, initiation sequences, and promoters useful for regulation of the expression of the nucleic acid sequences.

The nucleic acid can be cloned into the vector using any known molecular cloning methods in the art, including, for example, using restriction endonuclease sites and one or more selectable markers. In some embodiments, the nucleic acid is operably linked to a promoter. Varieties of promoters have been explored for gene expression in prokaryotic cells or eukaryotic cells (e.g., mammalian cells), and any of the promoters known in the art may be used in the present application. Promoters may be roughly categorized as constitutive promoters or regulated promoters, such as inducible promoters.

In some embodiments, the nucleic acid encoding the prodrugs described herein is operably linked to a constitutive promoter. Constitutive promoters allow heterologous genes (also referred to as transgenes) to be expressed constitutively in the host cells. Exemplary promoters contemplated herein include, but are not limited to, cytomegalovirus immediate-early promoter (CMV), human elongation factors-lalpha (hEFlu), ubiquitin C promoter (UbiC), phosphoglycerokinase promoter (PGK), simian virus 40 early promoter (SV40), chicken β-Actin promoter coupled with CMV early enhancer (CAGG), a Rous Sarcoma Virus (RSV) promoter, a polyoma enhancer/herpes simplex thymidine kinase (MC1) promoter, a beta actin (β-ACT) promoter, a “myeloproliferative sarcoma virus enhancer, negative control region deleted, d1587rev primer-binding site substituted (MND)” promoter. The efficiencies of such constitutive promoters on driving transgene expression have been widely compared in a huge number of studies. In some embodiments, the nucleic acid encoding the prodrugs described herein is operably linked to CMV promoter.

In some embodiments, the nucleic acid encoding the prodrugs described herein is operably linked to an inducible promoter. Inducible promoters belong to the category of regulated promoters. The inducible promoter can be induced by one or more conditions, such as a physical condition, microenvironment of the host cells, or the physiological state of the host cells, an inducer (i.e., an inducing agent), or a combination thereof. In some embodiments, the inducing condition does not induce the expression of endogenous genes in the host cell. In some embodiments, the inducing condition is selected from the group consisting of inducer, irradiation (such as ionizing radiation, light), temperature (such as heat), redox state, and the activation state of the host cell. In some embodiments, the inducible promoter can be an NFAT promoter, a TETON® promoter, or an NFκB promoter.

### Methods of Improving the Activity of IL-15

The present application also provided methods of improving the activity of IL-15Rα or a functional fragment and Fc fusion protein or IL-15 and IL-15Rα heterodimeric protein described herein. In some embodiments, the method comprises setting the peptide linker connecting the IL-15Rα or a functional fragment thereof and the Fc domain comprising more than 15 and less than 40 amino acid residues. In some embodiments, the peptide linker comprises 20 to 25 amino acid residues. In some embodiments, the peptide linker comprises 25 amino acid residues.

### Methods of Preparation

Also provided are methods of preparing any of the IL-15Rα and Fc fusion protein, the IL-15 and IL-15Rα heterodimeric protein described herein. Thus in some embodiments, there is provided a method of producing the IL-15Rα and Fc fusion protein, the IL-15 and IL-15Rα heterodimeric protein, comprising: (a) culturing a host cell (e.g., CHO cell, HEK 293 cell, Hela cell, or COS cell) comprising any of the nucleic acids or vectors encoding the IL-15Rα and Fc fusion protein, the IL-15 and IL-15Rα heterodimeric protein described herein under a condition effective to express the encoded prodrug; and (b) obtaining the expressed IL-15Rα and Fc fusion protein, IL-15 and IL-15Rα heterodimeric protein or prodrugs from said host cell. In some embodiments, the method of step (a) further comprises producing a host cell comprising the IL-15Rα and Fc fusion protein, the IL-15 and IL-15Rα heterodimeric protein described herein. The IL-15Rα and Fc fusion protein, the IL-15 and IL-15Rα heterodimeric protein described herein may be prepared using any methods known in the art or as described herein.

In some embodiments, the IL-15Rα and Fc fusion protein, the IL-15 and IL-15Rα heterodimeric protein described herein are expressed with eukaryotic cells, such as mammalian cells. In some embodiments, the IL-15Rα and Fc fusion protein, the IL-15 and IL-15Rα heterodimeric protein described herein are expressed with prokaryotic cells.

### 1. Recombinant Production in Prokaryotic Cells

### a) Vector Construction

Polynucleic acid sequences encoding the protein constructs of the present application can be obtained using standard recombinant techniques. Polynucleotides can be synthesized using nucleotide synthesizer or PCR techniques. Once obtained, sequences encoding the polypeptides are inserted into a recombinant vector capable of replicating and expressing heterologous polynucleotides in prokaryotic hosts. Many vectors that are available and known in the art can be used for the purpose of the present application. Selection of an appropriate vector will depend mainly on the size of the nucleic acids to be inserted into the vector and the particular host cell to be transformed with the vector. Each vector contains various components, depending on its function (amplification or expression of heterologous polynucleotide, or both) and its compatibility with the particular host cell in which it resides. The vector components generally include, but are not limited to: an origin of replication, a selection marker gene, a promoter, a ribosome binding site (RBS), a signal sequence, the heterologous nucleic acid insert and a transcription termination sequence.

In general, plasmid vectors containing replicon and control sequences which are derived from species compatible with the host cell are used in connection with these hosts. The vector ordinarily carries a replication site, as well as marking sequences which are capable of providing phenotypic selection in transformed cells. For example, E. coli is typically transformed using pBR322, a plasmid derived from an E. coli species. pBR322 contains genes encoding ampicillin (Amp) and tetracycline (Tet) resistance and thus provides easy means for identifying transformed cells. pBR322, its derivatives, or other microbial plasmids or bacteriophage may also contain, or be modified to contain, promoters which can be used by the microbial organism for expression of endogenous proteins. Examples of pBR322 derivatives used for expression of particular antibodies are described in detail in Carter et al., U.S. Pat. No. 5,648,237.

In addition, phage vectors containing replicon and control sequences that are compatible with the host microorganism can be used as transforming vectors in connection with these hosts. For example, bacteriophage such as GEM™-11 may be utilized in making a recombinant vector, which can be used to transform susceptible host cells such as E. coli LE392.

A promoter is an untranslated regulatory sequence located upstream (5′) to a cistron that modulates its expression. Prokaryotic promoters typically fall into two classes, inducible and constitutive. Inducible promoter is a promoter that initiates increased levels of transcription of the cistron under its control in response to changes in the culture condition, e.g. the presence or absence of a nutrient or a change in temperature.

A large number of promoters recognized by a variety of potential host cells are well known. The selected promoter can be operably linked to cistron DNA encoding the polypeptide by removing the promoter from the source DNA via restriction enzyme digestion and inserting the isolated promoter sequence into the vector of the present application. Both the native promoter sequence and many heterologous promoters may be used to direct amplification and/or expression of the target genes. In some embodiments, heterologous promoters are utilized, as they generally permit greater transcription and higher yields of expressed target gene as compared to the native target polypeptide promoter.

Promoters suitable for use with prokaryotic hosts include the PhoA promoter, the -galactamase and lactose promoter systems, a tryptophan (trp) promoter system and hybrid promoters such as the tac or the trc promoter. However, other promoters that are functional in bacteria (such as other known bacterial or phage promoters) are suitable as well. Their nucleic acid sequences have been published, thereby enabling a skilled worker operably to ligate them to cistrons encoding the target light and heavy chains (Siebenlist et al. (1980) Cell 20: 269) using linkers or adaptors to supply any required restriction sites.

In some embodiments, each cistron within the recombinant vector comprises a secretion signal sequence component that directs translocation of the expressed polypeptides across a membrane. In general, the signal sequence may be a component of the vector, or it may be a part of the target polypeptide DNA that is inserted into the vector. The signal sequence selected for the purpose of this application should be one that is recognized and processed (i.e., cleaved by a signal peptidase) by the host cell. For prokaryotic host cells that do not recognize and process the signal sequences native to the heterologous polypeptides, the signal sequence is substituted by a prokaryotic signal sequence selected, for example, from the group consisting of the alkaline phosphatase, penicillinase, Ipp, or heat-stable enterotoxin II (STII) leaders, LamB, PhoE, PelB, OmpA and MBP.

In some embodiments, the production of the protein construct according to the present application can occur in the cytoplasm of the host cell, and therefore does not require the presence of secretion signal sequences within each cistron. In some embodiments, polypeptide components are expressed, folded, and assembled to form the protein construct within the cytoplasm. Certain host strains (e.g., the E. coli trxB⁻ strains) provide cytoplasm conditions that are favorable for disulfide bond formation, thereby permitting proper folding and assembly of expressed protein subunits. See Proba and Pluckthun, Gene, 159:203 (1995).

### b) Prokaryotic Host Cells

Prokaryotic host cells suitable for expressing the proteins of the present application include Archaebacteria and Eubacteria, such as Gram-negative or Gram-positive organisms. Examples of useful bacteria include Escherichia (e.g., E. coli), Bacilli (e.g., B. subtilis), Enterobacteria, Pseudomonas species (e.g., P. aeruginosa), Salmonella typhimurium, Serratia marcescans, Klebsiella, Proteus, Shigella, Rhizobia, Vitreoscilla, or Paracoccus. In some embodiments, gram-negative cells are used. In some embodiments, E. coli cells are used as hosts for the application. Examples of E. coli strains include strain W3110 (Bachmann, Cellular and Molecular Biology, vol. 2 (Washington, D.C.: American Society for Microbiology, 1987), pp. 1190-1219; ATCC Deposit No. 27,325) and derivatives thereof, including strain 33D3 having genotype W3110 AfhuA (AtonA) ptr3 lac Iq lacL8 AompT A(nmpc-fepE) degP41 kan^{R}(U.S. Pat. No. 5,639,635). Other strains and derivatives thereof, such as E. coli 294 (ATCC 31,446), E. coli B, E. coli 1776 (ATCC 31,537) and E. coli RV308 (ATCC 31,608) are also suitable. These examples are illustrative rather than limiting. Methods for constructing derivatives of any of the above-mentioned bacteria having defined genotypes are known in the art and described in, for example, Bass et al., Proteins, 8:309-314 (1990). It is generally necessary to select the appropriate bacteria taking into consideration replicability of the replicon in the cells of a bacterium. For example, E. coli, Serratia, or Salmonella species can be suitably used as the host when well-known plasmids such as pBR322, pBR325, pACYC177, or pKN410 are used to supply the replicon.

Typically, the host cell should secrete minimal amounts of proteolytic enzymes, and additional protease inhibitors may desirably be incorporated in the cell culture.

### c) Protein Production

Host cells are transformed with the above-described expression vectors and cultured in conventional nutrient media modified as appropriate for inducing promoters, selecting transformants, or amplifying the genes encoding the desired sequences. Transformation means introducing DNA into the prokaryotic host so that the DNA is replicable, either as an extrachromosomal element or by chromosomal integrant. Depending on the host cell used, transformation is done using standard techniques appropriate to such cells. The calcium treatment employing calcium chloride is generally used for bacterial cells that contain substantial cell-wall barriers. Another method for transformation employs polyethylene glycol/DMSO. Yet another technique used is electroporation.

Prokaryotic cells used to produce the protein constructs of the present application are grown in media known in the art and suitable for culture of the selected host cells. Examples of suitable media include luria broth (LB) plus necessary nutrient supplements. In some embodiments, the media also contains a selection agent, chosen based on the construction of the expression vector, to selectively permit growth of prokaryotic cells containing the expression vector. For example, ampicillin is added to media for growth of cells expressing ampicillin resistant gene.

Any necessary supplements besides carbon, nitrogen and inorganic phosphate sources may also be included at appropriate concentrations introduced alone or as a mixture with another supplement or medium such as a complex nitrogen source. Optionally the culture medium may contain one or more reducing agents selected from the group consisting of glutathione, cysteine, cystamine, thioglycollate, dithioerythritol, and dithiothreitol. The prokaryotic host cells are cultured at suitable temperatures. For E. coli growth, for example, the preferred temperature ranges from about 20° C. to about 39° C., more preferably from about 25° C. to about 37° C., even more preferably at about 30° C. The pH of the medium may be any pH ranging from about 5 to about 9, depending mainly on the host organism. For E. coli, the pH is preferably from about 6.8 to about 7.4, and more preferably about 7.0.

If an inducible promoter is used in the expression vector of the present application, protein expression is induced under conditions suitable for the activation of the promoter. In one aspect of the present application, PhoA promoters are used for controlling transcription of the polypeptides. Accordingly, the transformed host cells are cultured in a phosphate-limiting medium for induction. Preferably, the phosphate-limiting medium is the C.R.A.P medium (see, e.g., Simmons et al., J. Immunol. Methods (2002), 263:133-147). A variety of other inducers may be used, according to the vector construct employed, as is known in the art.

The expressed protein constructs of the present application are secreted into and recovered from the periplasm of the host cells. Protein recovery typically involves disrupting the microorganism, generally by such means as osmotic shock, sonication, or lysis. Once cells are disrupted, cell debris or whole cells may be removed by centrifugation or filtration. The proteins may be further purified, for example, by affinity resin chromatography. Alternatively, proteins can be transported into the culture media and isolated therein. Cells may be removed from the culture and the culture supernatant being filtered and concentrated for further purification of the proteins produced. The expressed polypeptides can be further isolated and identified using commonly known methods such as polyacrylamide gel electrophoresis (PAGE) and Western blot assay.

Alternatively, protein production is conducted in large quantities by a fermentation process. Various large-scale fed-batch fermentation procedures are available for the production of recombinant proteins. Large-scale fermentations have at least 1000 liters of capacity, preferably about 1,000 to 100,000 liters of capacity. These fermentors use agitator impellers to distribute oxygen and nutrients, especially glucose (the preferred carbon/energy source). Small-scale fermentation refers generally to fermentation in a fermentor that is no more than approximately 100 liters in volumetric capacity and can range from about 1 liter to about 100 liters.

During the fermentation process, induction of protein expression is typically initiated after the cells have been grown under suitable conditions to a desired density, e.g., an ODsso of about 180-220, at which stage the cells are in the early stationary phase. A variety of inducers may be used, according to the vector construct employed, as is known in the art and described above. Cells may be grown for shorter periods prior to induction. Cells are usually induced for about 12-50 hours, although longer or shorter induction time may be used.

To improve the production yield and quality of the protein constructs of the present application, various fermentation conditions can be modified. For example, to improve the proper assembly and folding of the secreted polypeptides, additional vectors overexpressing chaperone proteins, such as Dsb proteins (DsbA, DsbB, DsbC, DsbD, or DsbG) or FkpA (a peptidylprolyl cis-, trans-isomerase with chaperone activity) can be used to co-transform the host prokaryotic cells. The chaperone proteins have been demonstrated to facilitate the proper folding and solubility of heterologous proteins produced in bacterial host cells. Chen et al. (1999) J Bio Chem 274:19601-19605; Georgiou et al., U.S. Pat. No. 6,083,715; Georgiou et al., U.S. Pat. No. 6,027,888; Bothmann and Pluckthun (2000) J. Biol. Chem. 275:17100-17105; Ramm and Pluckthun (2000) J. Biol. Chem. 275:17106-17113; Arie et al. (2001) Mol. Microbiol. 39:199-210.

To minimize proteolysis of expressed heterologous proteins (especially those that are proteolytically sensitive), certain host strains deficient for proteolytic enzymes can be used for the present application. For example, host cell strains may be modified to effect genetic mutation(s) in the genes encoding known bacterial proteases such as Protease III, OmpT, DegP, Tsp, Protease I, Protease Mi, Protease V, Protease VI and combinations thereof. Some E. coli protease-deficient strains are available and described in, for example, Joly et al. (1998), supra; Georgiou et al., U.S. Pat. No. 5,264,365; Georgiou et al., U.S. Pat. No. 5,508,192; Hara et al., Microbial Drug Resistance, 2:63-72 (1996).

E. coli strains deficient for proteolytic enzymes and transformed with plasmids overexpressing one or more chaperone proteins may be used as host cells in the expression system encoding the protein constructs of the present application.

### d) Protein Purification

The protein constructs produced herein are further purified to obtain preparations that are substantially homogeneous for further assays and uses. Standard protein purification methods known in the art can be employed. The following procedures are exemplary of suitable purification procedures: fractionation on immunoaffinity or ion-exchange columns, ethanol precipitation, reverse phase HPLC, chromatography on silica or on a cation-exchange resin such as DEAE, chromatofocusing, SDS-PAGE, ammonium sulfate precipitation, and gel filtration using, for example, Sephadex G-75.

In some embodiments, Protein A immobilized on a solid phase is used for immunoaffinity purification of the protein constructs comprising an Fc region of the present application. Protein A is a 42 kDa surface protein from Staphylococcus aureas which binds with a high affinity to Fc-containing constructs, e.g., IL-15Rα and Fc fusion protein, IL-15 and IL-15Rα heterodimeric protein or prodrugs described herein. Lindmark et al (1983) J. Immunol. Meth. 62:1-13. The solid phase to which Protein A is immobilized is preferably a column comprising a glass or silica surface, more preferably a controlled pore glass column or a silicic acid column. In some applications, the column has been coated with a reagent, such as glycerol, in an attempt to prevent nonspecific adherence of contaminants. The solid phase is then washed to remove contaminants non-specifically bound to the solid phase. Finally, the protein constructs of interest are recovered from the solid phase by elution.

### 2. Recombinant Production in Eukaryotic Cells

For eukaryotic expression, the vector components generally include, but are not limited to, one or more of the following, a signal sequence, an origin of replication, one or more marker genes, an enhancer element, a promoter, and a transcription termination sequence.

### a) Signal Sequence Component

A vector for use in a eukaryotic host may also be an insert that encodes a signal sequence or other polypeptide having a specific cleavage site at the N-terminus of the mature protein or polypeptide. The heterologous signal sequence selected preferably is one that is recognized and processed (i.e., cleaved by a signal peptidase) by the host cell. In mammalian cell expression, mammalian signal sequences as well as viral secretory leaders, for example, the herpes simplex gD signal, are available. The DNA for such precursor region is ligated in reading frame to DNA encoding the protein constructs of the present application.

### b) Origin of Replication

Generally, the origin of replication component is not needed for mammalian expression vectors (the SV40 origin may typically be used only because it contains the early promoter).

### c) Selection Gene Component

Expression and cloning vectors may contain a selection gene, also termed a selectable marker. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media, e.g., the gene encoding D-alanine racemase for Bacilli.

One example of a selection scheme utilizes a drug to arrest growth of a host cell. Those cells that are successfully transformed with a heterologous gene produce a protein conferring drug resistance and thus survive the selection regimen. Examples of such dominant selection use the drugs neomycin, mycophenolic acid and hygromycin.

Another example of suitable selectable markers for mammalian cells are those that enable the identification of cells competent to take up nucleic acid encoding the protein constructs of the present application, such as DHFR, thymidine kinase, metallothionein-I and -II, preferably primate metallothionein genes, adenosine deaminase, ornithine decarboxylase, etc.

For example, cells transformed with the DHFR selection gene are first identified by culturing all of the transformants in a culture medium that contains methotrexate (Mtx), a competitive antagonist of DHFR. An appropriate host cell when wild-type DHFR is employed is the Chinese hamster ovary (CHO) cell line deficient in DHFR activity (e.g., ATCC CRL-9096).

Alternatively, host cells (particularly wild-type hosts that contain endogenous DHFR) transformed or co-transformed with the polypeptide encoding-DNA sequences, wild-type DHFR protein, and another selectable marker such as aminoglycoside 3′-phosphotransferase (APH) can be selected by cell growth in medium containing a selection agent for the selectable marker such as an aminoglycosidic antibiotic, e.g., kanamycin, neomycin, or G418. See U.S. Pat. No. 4,965,199.

### d) Promoter Component

Expression and cloning vectors usually contain a promoter that is recognized by the host organism and is operably linked to the nucleic acid encoding the desired polypeptide sequences. Virtually all eukaryotic genes have an AT-rich region located approximately 25 to 30 based upstream from the site where transcription is initiated. Another sequence found 70 to 80 bases upstream from the start of the transcription of many genes is a CNCAAT region where N may be any nucleotide. At the 3′ end of most eukaryotic is an AATAAA sequence that may be the signal for addition of the poly A tail to the 3′ end of the coding sequence. All of these sequences may be inserted into eukaryotic expression vectors. Also see section “Vectors” above.

Polypeptide transcription from vectors in mammalian host cells is controlled, for example, by promoters obtained from the genomes of viruses such as polyoma virus, fowlpox virus, adenovirus (such as Adenovirus 2), bovine papilloma virus, avian sarcoma virus, cytomegalovirus, a retrovirus, hepatitis-B virus and most preferably Simian Virus 40 (SV40), from heterologous mammalian promoters, e.g., the actin promoter or an immunoglobulin promoter, from heat-shock promoters, provided such promoters are compatible with the host cell systems.

The early and late promoters of the SV40 virus are conveniently obtained as an SV40 restriction fragment that also contains the SV40 viral origin of replication. The immediate early promoter of the human cytomegalovirus is conveniently obtained as a HindIII E restriction fragment. A system for expressing DNA in mammalian hosts using the bovine papilloma virus as a vector is disclosed in U.S. Pat. No. 4,419,446. A modification of this system is described in U.S. Pat. No. 4,601,978. See also Reyes et al., Nature 297:598-601 (1982) on expression of human-interferon cDNA in mouse cells under the control of a thymidine kinase promoter from herpes simplex virus. Alternatively, the Rous Sarcoma Virus long terminal repeat can be used as the promoter.

### e) Enhancer Element Component

Transcription of a DNA encoding the protein constructs of the present application by higher eukaryotes is often increased by inserting an enhancer sequence into the vector. Many enhancer sequences are now known from mammalian genes (globin, elastase, albumin, α-fetoprotein, and insulin). Typically, however, one will use an enhancer from a eukaryotic cell virus. Examples include the SV40 enhancer on the late side of the replication origin (100-270 bp), the cytomegalovirus early promoter enhancer, the polyoma enhancer on the late side of the replication origin, and adenovirus enhancers. See also Yaniv, Nature 297:17-18 (1982) on enhancing elements for activation of eukaryotic promoters. The enhancer may be spliced into the vector at a position 5′ or 3′ to the polypeptide encoding sequence, but is preferably located at a site 5′ from the promoter.

### f) Transcription Termination Component

Expression vectors used in eukaryotic host cells (yeast, fungi, insect, plant, animal, human, or nucleated cells from other multicellular organisms) will also contain sequences necessary for the termination of transcription and for stabilizing the mRNA. Such sequences are commonly available from the 5′ and, occasionally 3′, untranslated regions of eukaryotic or viral DNAs or cDNAs. These regions contain nucleotide segments transcribed as polyadenylated fragments in the untranslated portion of the polypeptide-encoding mRNA. One useful transcription termination component is the bovine growth hormone polyadenylation region. See WO94/11026 and the expression vector disclosed therein.

### g) Selection and Transformation of Host Cells

Suitable host cells for cloning or expressing the DNA in the vectors herein include higher eukaryote cells described herein, including vertebrate host cells. Propagation of vertebrate cells in culture (tissue culture) has become a routine procedure. Examples of useful mammalian host cell lines are monkey kidney CV1 line transformed by SV40 (COS-7, ATCC CRL 1651); COS fibroblast-like cell lines derived from monkey kidney tissue; human embryonic kidney line (293 or 293 cells subcloned for growth in suspension culture, Graham et al., J. Gen Virol. 36:59 (1977)); baby hamster kidney cells (BHK, ATCC CCL 10); Chinese hamster ovary cells/-DHFR (CHO, Urlaub et al., Proc. Natl. Acad. Sci. USA 77:4216 (1980)); mouse sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TR1 cells (Mather et al., Annals N. Y. Acad. Sci. 383:44-68 (1982)); MRC 5 cells; FS4 cells; and a human hepatoma line (Hep G2).

Host cells are transformed with the above-described expression or cloning vectors for protein construct production and cultured in conventional nutrient media modified as appropriate for inducing promoters, selecting transformants, or amplifying the genes encoding the desired sequences.

### h) Culturing the Host Cells

The host cells used to produce the protein constructs of the present application may be cultured in a variety of media. Commercially available media such as Ham's F10 (Sigma), Minimal Essential Medium ((MEM), (Sigma), RPMI-1640 (Sigma), and Dulbecco's Modified Eagle's Medium ((DMEM), Sigma) are suitable for culturing the host cells. In addition, any of the media described in Ham et al., Meth. Enz. 58:44 (1979), Barnes et al., Anal. Biochem. 102:255 (1980), U.S. Pat. Nos. 4,767,704; 4,657,866; 4,927,762; 4,560,655; or 5,122,469; WO 90/03430; WO 87/00195; or U.S. Pat. Re. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as GENTAMYCIN™ drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression and will be apparent to the ordinarily skilled artisan.

### i) Protein Purification

When using recombinant techniques, the protein constructs of the present application can be produced intracellularly, in the periplasmic space, or directly secreted into the medium. If the protein construct is produced intracellularly, as a first step, the particulate debris, either host cells or lysed fragments, are removed, for example, by centrifugation or ultrafiltration. Carter et al., Bio/Technology 10:163-167 (1992) describe a procedure for isolating antibodies that are secreted to the periplasmic space of E. coli. Briefly, cell paste is thawed in the presence of sodium acetate (pH 3.5), EDTA, and phenylmethylsulfonylfluoride (PMSF) over about 30 min. Cell debris can be removed by centrifugation. Where the protein construct is secreted into the medium, supernatants from such expression systems are generally first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants.

The protein composition prepared from the cells can be purified using, for example, hydroxylapatite chromatography, gel electrophoresis, dialysis, and affinity chromatography, with affinity chromatography being the preferred purification technique. The suitability of protein A as an affinity ligand depends on the species and isotype of any immunoglobulin Fc domain that is present in the Fc-containing protein construct. Protein A can be used to purify Fe-containing proteins based on human immunoglobulins containing 1, 2, or 4 heavy chains (Lindmark et al., J. Immunol. Meth. 62:1-13 (1983)). Protein G is recommended for all mouse isotypes and for human 3 (Guss et al., EMBO J. 5:15671575 (1986)). The matrix to which the affinity ligand is attached is most often agarose, but other matrices are available. Mechanically stable matrices such as controlled pore glass or poly(styrene-divinyl) benzene allow for faster flow rates and shorter processing times than can be achieved with agarose. Where the protein construct comprises a CH3 domain, the Bakerbond ABXTMresin (J. T. Baker, Phillipsburg, N.J.) is useful for purification. Other techniques for protein purification such as fractionation on an ion-exchange column, ethanol precipitation, Reverse Phase HPLC, chromatography on silica, chromatography on heparin SEPHAROSE™ chromatography on an anion or cation exchange resin (such as a polyaspartic acid column), chromatofocusing, SDS-PAGE, and ammonium sulfate precipitation are also available depending on the protein construct to be recovered.

Following any preliminary purification step(s), the mixture comprising the protein constructs of interest and contaminants may be subjected to low pH hydrophobic interaction chromatography using an elution buffer at a pH between about 2.5-4.5, preferably performed at low salt concentrations (e.g., from about 0-0.25M salt).

### Pharmaceutical Compositions

Further provided are pharmaceutical compositions comprising any of the IL-15Rα and Fc fusion protein, the IL-15 and IL-15Rα heterodimeric protein described herein, and optionally a pharmaceutically acceptable carrier. Pharmaceutical compositions can be prepared by mixing a prodrug described herein having the desired degree of purity with optional pharmaceutically acceptable carriers, excipients or stabilizers (Remington's Pharmaceutical Sciences 16th edition, Osol, A. Ed. (1980)), in the form of lyophilized formulations or aqueous solutions.

A reconstituted formulation can be prepared by dissolving a lyophilized protein in a diluent such that the protein is dispersed throughout. Exemplary pharmaceutically acceptable (safe and non-toxic for administration to a human) diluents suitable for use in the present application include, but are not limited to, sterile water, bacteriostatic water for injection (BWFI), a pH buffered solution (e.g., phosphate-buffered saline), sterile saline solution, Ringer's solution or dextrose solution, or aqueous solutions of salts and/or buffers.

In some embodiments, the pharmaceutical composition comprises a homogeneous population of IL-15Rα and Fc fusion protein, IL-15 and IL-15Rα heterodimeric protein described herein. A homogeneous population means the proteins are exactly the same to each other, e.g., same fusion protein configuration, same heterodimeric protein configuration, same IL-15 prodrug configuration, same IL-15 cytokine, same IL-15Rα_sushi domain, same masking polypeptides, same cleavable moiety, same non-cleavable linker if any, and same Fc domain. In some embodiments, at least about 70% (such as at least about any of 75%, 80%, 85%, 90%, 95%, 96%, 97%, 98%, or 99%) of the protein in the pharmaceutical composition are homogeneous.

The pharmaceutical composition is preferably to be stable, in which the proteins contained within essentially retain their physical and chemical stability and integrity upon storage. Various analytical techniques for measuring protein stability are available in the art and are reviewed in Peptide and Protein Drug Delivery, 247-301, Vincent Lee Ed., Marcel Dekker, Inc., New York, N.Y., Pubs. (1991) and Jones, A. Adv. Drug Delivery Rev. 10: 29-90 (1993). Stability can be measured at a selected temperature for a selected time period. For rapid screening, the formulation may be kept at 40° C. for 2 weeks to 1 month, at which time stability is measured. For example, the extent of aggregation during storage can be used as an indicator of protein stability.

In some embodiments, the pharmaceutical composition has a shelf life of at least about 15 days, such as at least about any of 20 days, 1 month, 2 months, 3 months, 6 months, 1 year, 2 years, 3 years, or longer, for example, at about 2-25° C., such as about 2-8° C. As used herein, “shelf life” means that the storage period during which an active ingredient such as a therapeutic protein (e.g., IL-15Rα and Fc fusion protein, IL-15 and IL-15Rα heterodimeric protein or prodrugs described herein) in a pharmaceutical formulation has minimal degradation (e.g., not more than about 5% degradation, such as not more than about 4%, 3%, or 2% degradation) when the pharmaceutical formulation is stored under specified storage conditions, for example, 2-8° C. Exemplary techniques for assessing protein or formulation stability include size-exclusion chromatography (SEC)-HPLC to detect, e.g., aggregation, reverse phase (RP)-HPLC to detect, e.g. protein fragmentation, ion exchange-HPLC to detect, e.g., changes in the charge of the protein, mass spectrometry, fluorescence spectroscopy, circular dichroism (CD) spectroscopy, Fourier transform infrared spectroscopy (FT-IR), and Raman spectroscopy to detect protein conformational changes. All of these techniques can be used singly or in combination to assess the degradation of the protein in the pharmaceutical formulation and determine the shelf life of that formulation.

Acceptable carriers, excipients, or stabilizers are nontoxic to recipients at the dosages and concentrations employed and include buffers, antioxidants including ascorbic acid, methionine, Vitamin E, sodium metabisulfite; preservatives, isotonicifiers (e.g., sodium chloride), stabilizers, metal complexes (e.g. Zn-protein complexes); chelating agents such as EDTA and/or non-ionic surfactants.

Examples of physiologically acceptable carriers include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugars such as sucrose, mannitol, trehalose or sorbitol; salt-forming counterions such as sodium; metal complexes (e.g. Zn-protein complexes); and/or nonionic surfactants such as TWEEN™, polyethylene glycol (PEG), and PLURONICS™.

Buffers are used to control the pH in a range which optimizes the therapeutic effectiveness, especially if stability is pH dependent. Suitable buffering agents for use in the present application include both organic and inorganic acids and salts thereof. For example, citrate, phosphate, succinate, tartrate, fumarate, gluconate, oxalate, lactate, acetate. Additionally, buffers may comprise histidine and trimethylamine salts such as Tris.

Preservatives are added to retard microbial growth. The addition of a preservative may, for example, facilitate the production of a multi-use (multiple-dose) formulation. Suitable preservatives for use in the present application include octadecyldimethylbenzyl ammonium chloride; hexamethonium chloride; benzalkonium halides (e.g., chloride, bromide, iodide), benzethonium chloride; thimerosal, phenol, butyl or benzyl alcohol; alkyl parabens such as methyl or propyl paraben; catechol; resorcinol; cyclohexanol, 3-pentanol, and m-cresol.

Tonicity agents, sometimes known as “stabilizers” are present to adjust or maintain the tonicity of liquid in a composition. When used with large, charged biomolecules such as proteins, they are often termed “stabilizers” because they can interact with the charged groups of the amino acid side chains, thereby lessening the potential for inter and intra-molecular interactions. Tonicity agents can be present in any amount between 0.1% to 25% by weight, preferably 1% to 5%, taking into account the relative amounts of the other ingredients. Preferred tonicity agents include polyhydric sugar alcohols, preferably trihydric or higher sugar alcohols, such as glycerin, erythritol, arabitol, xylitol, sorbitol and mannitol.

Additional excipients include agents which can serve as one or more of the following: (1) bulking agents, (2) solubility enhancers, (3) stabilizers and (4) and agents preventing denaturation or adherence to the container wall. Such excipients include: polyhydric sugar alcohols (enumerated above); amino acids such as alanine, glycine, glutamine, asparagine, histidine, arginine, lysine, ornithine, leucine, 2-phenylalanine, glutamic acid, threonine, etc.; organic sugars or sugar alcohols such as sucrose, lactose, lactitol, trehalose, stachyose, mannose, sorbose, xylose, ribose, ribitol, myoinisitose, myoinisitol, galactose, galactitol, glycerol, cyclitols (e.g., inositol), polyethylene glycol; sulfur containing reducing agents, such as urea, glutathione, thioctic acid, sodium thioglycolate, thioglycerol, α-monothioglycerol and sodium thio sulfate; low molecular weight proteins such as human serum albumin, bovine serum albumin, gelatin or other immunoglobulins; hydrophilic polymers such as polyvinylpyrrolidone; monosaccharides (e.g., xylose, mannose, fructose, glucose; disaccharides (e.g., lactose, maltose, sucrose); trisaccharides such as raffinose; and polysaccharides such as dextrin or dextran.

Non-ionic surfactants or detergents (also known as “wetting agents”) are present to help solubilize the proteins as well as to protect the proteins against agitation-induced aggregation, which also permits the formulation to be exposed to shear surface stress without causing denaturation of the active proteins.

Suitable non-ionic surfactants include polysorbates (20, 40, 60, 65, 80, etc.), polyoxamers (184, 188, etc.), PLURONIC® polyols, TRITON®, polyoxyethylene sorbitan monoethers (TWEEN®-20, TWEEN®-80, etc.), lauromacrogol 400, polyoxyl 40 stearate, polyoxyethylene hydrogenated castor oil 10, 50 and 60, glycerol monostearate, sucrose fatty acid ester, methyl cellulose and carboxymethyl cellulose. Anionic detergents that can be used include sodium lauryl sulfate, dioctyle sodium sulfosuccinate and dioctyl sodium sulfonate. Cationic detergents include benzalkonium chloride or benzethonium chloride.

For the pharmaceutical compositions to be used for in vivo administration, they must be sterile. The pharmaceutical composition may be rendered sterile by filtration through sterile filtration membranes. The pharmaceutical compositions herein generally are placed into a container having a sterile access port, for example, an intravenous solution bag or vial having a stopper pierceable by a hypodermic injection needle.

Sustained-release preparations may be prepared. Suitable examples of sustained-release preparations include semi-permeable matrices of solid hydrophobic polymers containing the antagonist, which matrices are in the form of shaped articles, e.g. films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and Ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid.

The pharmaceutical compositions herein may also contain more than one active compound as necessary for the particular indication being treated, preferably those with complementary activities that do not adversely affect each other. Such molecules are suitably present in combination in amounts that are effective for the purpose intended.

The active ingredients may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 18th edition.

In some embodiments, the pharmaceutical composition is contained in a single-use vial, such as a single-use sealed vial. In some embodiments, the pharmaceutical composition is contained in a multi-use vial. In some embodiments, the pharmaceutical composition is contained in bulk in a container. In some embodiments, the pharmaceutical composition is cryopreserved.

### Methods of Treating Diseases

Further provided are methods of treating a subject with or at risk of developing a disease or disorder, such as proliferative disease, a tumorous disease, an inflammatory disease, an immunological disorder, an autoimmune disease, an infectious disease, a viral disease, an allergic reaction, a parasitic reaction, or graft-versus-host disease. The methods administering to a subject in need thereof an effective amount of an activatable prodrug as disclosed herein that is typically administered as a pharmaceutical composition, wherein the prodrug is activated upon cleavage by an enzyme. In some embodiments, the method further comprises selecting a subject with or at risk of developing such a disease or disorder. In some embodiments, the prodrug is activated in a tumor microenvironment. The prodrug is therapeutically active after it has cleaved from the masking polypeptides. Thus, in some embodiments, the active agent is the cleavage product. In some embodiments, the prodrugs can be used to treat a disease depending on the antigen bound by the antigen-binding domain.

In some embodiments, there is provided a method of treating a disease (e.g., a tumor, a viral infection, or a bacterial infection) in an individual (e.g., human), comprising administering to the individual an effective amount of any of the IL-15Rα and Fc fusion protein, the IL-15 and IL-15Rα heterodimeric protein described herein or pharmaceutical compositions thereof. In some embodiments, the IL-15Rα and Fc fusion protein, the IL-15 and IL-15Rα heterodimeric protein (or a pharmaceutical composition thereof) is administered intravenously, intramuscularly, or subcutaneously. In some embodiments, the method of treatment further comprises administering an additional therapeutic agent in combination with (before, after, or concurrently with) the prodrug. The additional agent may be an antibody or antigen-binding fragment thereof, a small molecule drug, or other types of therapeutic drug.

In some embodiments, the IL-15Rα and Fc fusion protein, the IL-15 and IL-15Rα heterodimeric protein is used to treat a cancer or tumor in a subject comprises administering to the subject an effective amount of the IL-15Rα and Fc fusion protein, the IL-15 and IL-15Rα heterodimeric protein. In some embodiments, as used herein, the term “tumor or cancer” refers to all types of cancer, neoplasm or malignant tumors found in mammals, including leukemias, lymphomas, melanomas, neuroendocrine tumors, carcinomas and sarcomas. Exemplary cancers that may be treated with a masked cytokine, pharmaceutical composition, or method provided herein, include lymphoma, sarcoma, bladder cancer, bone cancer, brain tumor, cervical cancer, colon cancer, esophageal cancer, gastric cancer, head and neck cancer, kidney cancer, myeloma, thyroid cancer, leukemia, prostate cancer, breast cancer (e.g. triple negative, ER positive, ER negative, chemotherapy resistant, Herceptin resistant, HER2 positive, doxorubicin resistant, tamoxifen resistant, ductal carcinoma, lobular carcinoma, primary, metastatic), ovarian cancer, pancreatic cancer, liver cancer (e.g. hepatocellular carcinoma), lung cancer (e.g. nonsmall cell lung carcinoma, squamous cell lung carcinoma, adenocarcinoma, large cell lung carcinoma, small cell lung carcinoma, carcinoid, sarcoma), glioblastoma multiforme, glioma, melanoma, prostate cancer, castration-resistant prostate cancer, breast cancer, triple negative breast cancer, glioblastoma, ovarian cancer, lung cancer, squamous cell carcinoma (e.g., head, neck, or esophagus), colorectal cancer, leukemia, acute myeloid leukemia, lymphoma, B cell lymphoma, or multiple myeloma. Additional examples include, cancer of the thyroid, endocrine system, brain, breast, cervix, colon, head & neck, esophagus, liver, kidney, lung, non-small cell lung, melanoma, mesothelioma, ovary, sarcoma, stomach, uterus or Medulloblastoma, Hodgkin's Disease, Non-Hodgkin's Lymphoma, multiple myeloma, neuroblastoma, glioma, glioblastoma multiforme, ovarian cancer, rhabdomyosarcoma, primary thrombocytosis, primary macroglobulinemia, primary brain tumors, cancer, malignant pancreatic insulanoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, Paget's Disease of the Nipple, Phyllodes Tumors, Lobular Carcinoma, Ductal Carcinoma, cancer of the pancreatic stellate cells, cancer of the hepatic stellate cells, or prostate cancer.

In some embodiments, the IL-15Rα and Fc fusion protein, the IL-15 and IL-15Rα heterodimeric protein is used to treat a bacterial infection such as sepsis. In some embodiments, the bacteria causing the bacterial infection are drug-resistant bacteria. In some embodiments, the antigen-binding moiety binds to a bacterial antigen.

In some embodiments, the IL-15Rα and Fc fusion protein, the IL-15 and IL-15Rα heterodimeric protein is used to treat a viral infection. In some embodiments, the virus causing the viral infection is hepatitis C (HCV), hepatitis B (HBV), human immunodeficiency vims (HIV), a human papilloma virus (HPV). In some embodiments, the antigen-binding moiety binds to a viral antigen.

Administration of the prodrug described herein or pharmaceutical compositions thereof may be carried out in any convenient manner, including by injection or transfusion. The route of administration is in accordance with known and accepted methods, such as by single or multiple bolus or infusion over a long period of time in a suitable manner. The prodrug or pharmaceutical compositions thereof may be administered to a patient orally, subcutaneously, intravenously, intracerebrally, intranasally, transdermally, intraperitoneally, intramuscularly, intrapulmonarily, vaginally, rectally, intraocularly, topically, transarterially, intradermally, intranodally, intraputaminally, or intramedullary, intrathecally, intraventricularly, intracerebrally, intraspinally, intrathecially, ntralesionally, or intraocularly. In some embodiments, the prodrug or pharmaceutical composition thereof is administered systemically. In some embodiments, the IL-15Rα and Fe fusion protein, the IL-15 and IL-15Rα heterodimeric protein or pharmaceutical composition thereof is administered to an individual by infusion, such as intravenous infusion. Infusion techniques for immunotherapy are known in the art (see, e.g., Rosenberg et al., New Eng. J. of Med. 319: 1676 (1988)). In some embodiments, the prodrug or pharmaceutical composition thereof is administered to an individual by intradermal or subcutaneous (i.e., beneath the skin) injection. For subcutaneous injections, the prodrug or pharmaceutical composition thereof may be injected using a syringe.

However, other devices for administration of the prodrug or pharmaceutical composition thereof are available such as injection devices; injector pens; auto-injector devices, needleless devices; and subcutaneous patch delivery systems. In some embodiments, the prodrug or pharmaceutical composition thereof is administered by intravenous injection. In some embodiments, the prodrug or pharmaceutical composition thereof is injected directly into the brain or spine. In some embodiments, the prodrug or pharmaceutical composition thereof is administered by sustained release or extended-release means.

Dosages and desired drug concentration of pharmaceutical compositions of the present application may vary depending on the particular use envisioned. The determination of the appropriate dosage or route of administration is well within the skill of an ordinary artisan. Animal experiments provide reliable guidance for the determination of effective doses for human therapy. Interspecies scaling of effective doses can be performed following the principles laid down by Mordenti, J. and Chappell, W. “The Use of Interspecies Scaling in Toxicokinetics,” In Toxicokinetics and New Drug Development, Yacobi et al., Eds, Pergamon Press, New York 1989, pp. 42-46.

When in vivo administration of the prodrug or pharmaceutical composition thereof are used, the dosage amounts may vary depending upon the route of administration and mammal type. It is within the scope of the present application that different formulations will be effective for different treatments and different disorders, and that administration intended to treat a specific organ or tissue may necessitate delivery in a manner different from that to another organ or tissue. Moreover, dosages may be administered by one or more separate administrations, or by continuous infusion. For repeated administrations over several days or longer, depending on the condition, the treatment is sustained until a desired suppression of disease symptoms occurs. However, other dosage regimens may be useful. The progress of this therapy is easily monitored by conventional techniques and assaysl.

In some embodiments, the IL-15Rα and Fe fusion protein, the IL-15 and IL-15Rα heterodimeric protein or pharmaceutical composition thereof is administered for a single time (e.g., bolus injection). In some embodiments, the IL-15Rα and Fc fusion protein, the IL-15 and IL-15Rα heterodimeric protein or pharmaceutical composition thereof is administered for multiple times (such as any of 2, 3, 4, 5, 6, or more times). If multiple administrations, they may be performed by the same or different routes and may take place at the same site or at alternative sites. The prodrug or pharmaceutical composition thereof may be administered daily to once per year. The interval between administrations can be about any one of 24 hours to a year. Intervals can also be irregular (e.g. following tumor progression). In some embodiments, there is no break in the dosing schedule. The optimal dosage and treatment regime for a particular patient can readily be determined by one skilled in the art of medicine by monitoring the patient for signs of disease and adjusting the treatment accordingly.

In some embodiments, the IL-15Rα and Fc fusion protein, the IL-15 and IL-15Rα heterodimeric protein or pharmaceutical composition thereof is administered in split doses, such as about any one of 2, 3, 4, 5, or more doses. In some embodiments, the split doses are administered over about a week, a month, 2 months, 3 months, or longer. In some embodiments, the dose is equally split. In some embodiments, the split doses are about 20%, about 30% and about 50% of the total dose. In some embodiments, the interval between consecutive split doses is about 1 day, 2 days, 3 days, 1 week, 2 weeks, 3 weeks, a month, 3 months, 6 months, or longer. For repeated administrations over several days or longer, depending on the condition, the treatment is sustained until a desired suppression of disease symptoms occurs. However, other dosage regimens may be useful. The progress of this therapy is easily monitored by conventional techniques and assays.

### Articles of Manufacture and Kits

Further provided are kits, unit dosages, and articles of manufacture comprising any of the prodrugs described herein. In some embodiments, a kit is provided which contains any one of the prodrug compositions described herein and preferably provides instructions for its use, such as for use in the treatment of the disorders described herein (e.g., tumor).

Kits of the application include one or more containers comprising a prodrug described herein, e.g., for treating a disease. For example, the instructions comprise a description of administration of the prodrug to treat a disease, such as a tumor. The kit may further comprise a description of selecting an individual (e.g., human) suitable for treatment based on identifying whether that individual has the disease and the stage of the disease. The instructions relating to the use of the prodrug generally include information as to dosage, dosing schedule, and route of administration for the intended treatment. The containers may be unit doses, bulk packages (e.g., multi-dose packages) or sub-unit doses. Instructions supplied in the kits of the application are typically written instructions on a label or package insert (e.g., a paper sheet included in the kit), but machine-readable instructions (e.g., instructions carried on a magnetic or optical storage disk) are also acceptable. The kits of the present application are in suitable packaging. Suitable packaging includes, but is not limited to, vials, bottles, jars, flexible packaging (e.g., sealed Mylar or plastic bags), and the like. Also contemplated are packages for use in combination with a specific device, such as an infusion device such as a minipump. A kit may have a sterile access port (for example, the container may be an intravenous solution bag or a vial having a stopper pierceable by a hypodermic injection needle). At least one active agent in the composition is the IL-15Rα and Fc fusion protein, the IL-15 and IL-15Rα heterodimeric protein as described herein. The container may further comprise a second pharmaceutically active agent. The kits may optionally provide additional components such as buffers and interpretive information. Normally, the kit comprises a container and a label or package insert(s) on or associated with the container.

The present application thus also provides articles of manufacture, which include vials (such as sealed vials), bottles, jars, flexible packaging, and the like. The article of manufacture can comprise a container and a label or package insert on or associated with the container. Suitable containers include, for example, bottles, vials, syringes, etc. The containers may be formed from a variety of materials such as glass or plastic. Generally, the container holds a composition which is effective for treating a disease or disorder (such as a tumor) described herein, and may have a sterile access port (for example, the container may be an intravenous solution bag or a vial having a stopper pierceable by a hypodermic injection needle). The label or package insert indicates that the composition is used for treating the particular condition in an individual. The label or package insert will further comprise instructions for administering the composition to the individual. The label may indicate directions for reconstitution and/or use. The container holding the pharmaceutical composition may be a multi-use vial, which allows for repeat administrations (e.g., from 2-6 administrations) of the reconstituted formulation. Package insert refers to instructions customarily included in commercial packages of therapeutic products that contain information about the indications, usage, dosage, administration, contraindications and/or warnings concerning the use of such therapeutic products. Additionally, the article of manufacture may further comprise a second container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

The kits or article of manufacture may include multiple unit doses of the pharmaceutical composition and instructions for use, packaged in quantities sufficient for storage and use in pharmacies, for example, hospital pharmacies and compounding pharmacies.

### EXAMPLES

The examples below are intended to be purely exemplary of the application and should therefore not be considered to limit the application in any way. The following examples and detailed descriptions are offered by way of illustration and not by way of limitation.

### Example 1: Construction, Expression, and Purification of Fc-IL-15/Fc-IL-15Rα_Sushi Heterodimeric Proteins

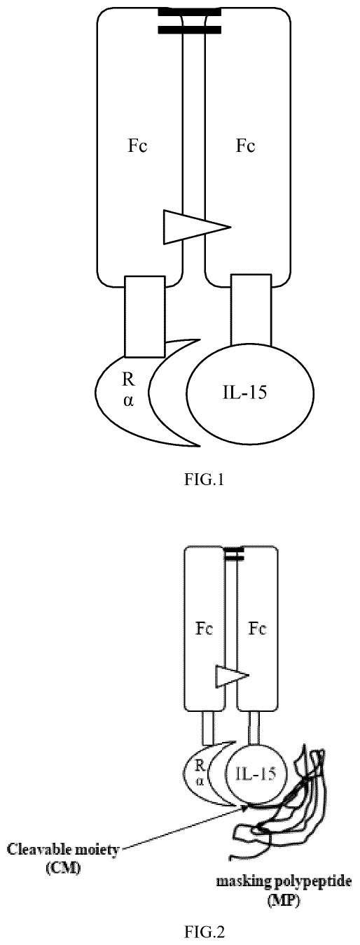

In order to increase the half-life of IL-15/IL-15Rα_sushi complex, the Fc-IL-15/Fc-IL-15Rα sushi heterodimeric proteins were generated by linking IL-15 to the first Fc domain and the IL-15Rα_sushi to the second Fc domain, respectively, depending on FcRn-mediated recycling to prolong the half-life. In order to reduce the toxicity of the heterodimeric protein, a form of Fc-IL-15/Fc-IL-15Rα sushi heterodimeric protein (hereinafter referred to as IL-15 prodrug) can be designed in the example by linking a masking polypeptide (MP) through a cleavable moiety (CM). In the exemplary IL-15 prodrug, the masking polypeptide (MP) shields IL-15 and reduces the activity of IL-15 in non-target tissue, and the cleavable moiety (CM) comprises a substrate sequence of the protease (e.g., MMP2 or MMP9) at the target tissue (e.g., tumor) for release of the masking polypeptide (MP). Once the masking polypeptide gets released off from the IL-15 prodrug at the target tissue, the protein gets activated and fully exhibits its activity. The constructs of Fc-IL-15/Fc-IL-15Rα_sushi heterodimeric proteins and prodrug forms thereof were shown in Table 1. FIG. 1 and FIG. 2 were exemplary schematic drawings illustrating an FEc-IL-15/Fc-IL-15Rα_sushi heterodimeric protein and prodrug form thereof, respectively. All the proteins were constructed and recombinantly expressed in HEK293 cells.

Plasmid construction: Nucleotide sequences for masking polypeptide, IL-15 and IL-15Rα_sushi was commercially synthesized (Genscripts USA) and digested with restriction enzymes correspondingly. The human IgG1 Fc(hole) or Fc(knob) was PCR amplified and digested with restriction enzymes correspondingly. The cleavable moiety CM was synthesized in a single forward and a single reverse nucleotide chain with corresponding restriction enzyme sites in both the 5′ and in the 3′ ends after annealing at 50° C. All synthesized gene fragments and PCR fragments were purified with commercial kits after running with 1% agarose gel and ligated into predigested plasmid pcDNA3.1 (Invitrogen) with T4 ligation kit. The ligation products were transformed into competent cells. After transformation and plating, colonies were picked up and grown at 37° C. overnight in LB media containing carbenicillin. The recombinant plasmids were extracted using commercial kit (Qiagen, Cat number 27104) and sequenced using both T7-forward and BGH reverse primers. The whole coding sequence was verified by DNA sequencing.

The exemplary sequences of non-cleavable linkers (lk, lk1, lk2, lk3, and lk5) were shown in Table 2 and the exemplary sequence of cleavable moiety CM1 was shown in Table3, the exemplary sequences of human IL-15 (mature form or precursor form) and IL-15 Rα_sushi (long version from or short version form) were shown in Table 4, the exemplary sequences of human IgG1 Fc(hole) and Fc(knob) were shown in Table 5, the exemplary sequences of masking polypeptides (MP) were shown in Table 6, and the exemplary sequences of Fc-IL-15/Fc-IL-15Rα_sushi heterodimeric proteins and prodrug form thereof were shown in Table 7. In Table 7, the human IL-15 or IL-15Rα_sushi domain is italicized, the non-cleavable linker is bolded, the cleavable moiety is underlined with single line, the masking polypeptide is underlined with d̅o̅u̅b̅l̅e̅ l̅i̅n̅e̅s̅, and the introduced restriction enzyme recognition sites is underlined with dotted line.

The linker between Fc and IL-15Rα_sushi:

Choosing an appropriate linker to connect protein domains is crucial in fusion protein engineering. The peptide linker not only provides the spatial distance between the domains of the fusion protein and allows them to fold independently but also directly affects the structural stability and functional properties of the fusion protein. Here, the influence of the flexibility and length of the linker on the biological activity of the Fc-IL-15/Fc-IL-15Rα_sushi heterodimeric proteins and IL-15 prodrugs was examined.

In some embodiments, the IL-15Rα_sushi and Fc were linked through a peptide linker.

In some embodiments, the linker included for example GSG and (GGGGS)n, wherein n was an integer of at least 1 (and optionally from 1 to 5) that allowed the two domains with sufficient length and flexibility to allow each domain to retain its biological function.

As shown in Table 1, the Group 1 were exemplary Fc-IL-15/Fc-IL-15Rα_sushi heterodimeric proteins without masking polypeptide SB1902-C1 and SB1902-C1-variant1, the only difference between SB1902-C1 and SB1902-C1-variant1 is the linker between Fc and IL-15Rα_sushi, SB1902-C1 has the linker “lk5” (GGGGS)₅ between Fc and IL-15Rα_sushi, and SB1902-C1-variant1 has the linker “lk” (GSG) between Fc and IL-15Rα_sushi.

The Group 2 were exemplary IL-15 prodrugs with the masking polypeptide MP80: SB1902-C2 and SB1902-C2-variant0, the only difference between SB1902-C2 and SB1902-C2-variant0 is the linker between Fc and IL-15Rα_sushi. SB1902-C2 has the linker “lk5” (GGGGS)₅ between Fc and IL-15Rα_sushi, and SB1902-C2-variant has the linker “lk” (GSG) between Fc and IL-15Rα_sushi.

The Group 3 were exemplary IL-15 prodrugs with the masking polypeptide MP163: SB1902-C9-variant1, SB1902-C9-variant2 and SB1902-C9-variant3, the only difference among SB1902-C9-variant1, SB1902-C9-variant2 and SB1902-C9-variant3 is the linker between Fc and IL-C5Rα_sushi. SB1902-C9-variant1 has the linker “lk5” (GGGGS)₅, SB1902-C9-variant2 has the linker “lk3” (GGGGS)₃ between Fc and IL-5Rα_sushi and SB1902-C9-variant3 has the linker “lk1” (GGGGS)₁ between Fc and IL-15Rα_sushi.

TABLE 1
Fc-IL-15/Fc-IL-15Rα sushi heterodimeric
proteins and prodrug form thereof

| | Construct | |
|---|---|---|
| Group | name | Construct description |
| Group 1 | SB1902-C1 | hIgG Fc(hole)-lk1-IL15 |
| | | hIgG Fc(knob)-lk5-IL15Rα_sushi |
| | SB1902-C1- | hIgG Fc(hole)-lk1-IL15 |
| | variant1 | hIgG Fc(knob)-lk-IL15Rα_sushi |
| Group 2 | SB1902-C2 | hIgG Fc(hole)-lk1-IL15-lk2-CM1-lk2-MP80 |
| | | hIgG Fc(knob)-lk5-IL15Rα_sushi |
| | SB1902-C2- | hIgG Fc(hole)-lk1-IL15-lk2-CM1-lk2-MP80 |
| | variant0 | hIgG Fc(knob)-lk-IL15Rα_sushi |
| Group 3 | SB1902-C9- | hIgG Fc(hole)-lk5-IL15Rα_sushi |
| | variant1 | hIgG Fc(knob)-lk1-IL15-lk2-CM1-lk2-MP163 |
| | SB1902-C9- | hIgG Fc(hole)-lk3-IL15Rα_sushi |
| | variant2 | hIgG Fc(knob)-lk1-IL15-lk2-CM1-lk2-MP163 |
| | SB1902-C9- | hIgG Fc(hole)-lk1-IL15Rα_sushi |
| | variant3 | hIgG Fc(knob)-lk1-IL15-lk2-CM1-lk2-MP163 |

TABLE 2

| Peptide Linker | SEQ ID NO. | Sequence |
|---|---|---|
| lk | 1 | GSG |
| lk1 | 2 | (G4S)1 |
| lk2 | 3 | (G4S)2 |
| lk3 | 4 | (G4S)3 |
| lk5 | 5 | (G4S)5 |

TABLE 3

| Cleavable | | |
|---|---|---|
| moiety | SEQ ID NO. | Sequence |
| CM1 | 6 | MVPSALTASG |

TABLE 4

| | SEQ ID NO. | Sequence |
|---|---|---|
| Human IL-15 (mature | 7 | NWVNVISDLKKIEDLIQSMHIDATLYTESDVHPSCKVTA |
| form) | | MKCFLLELQVISLESGDASIHDTVENLIILANNSLSSNGNV |
| | | TESGCKECEELEEKNIKEFLQSFVHIVQMFINTS |
| Human IL-15 | 8 | MRISKPHLRSISIQCYLCLLLNSHFLTEAGIHVFILGCFSAG |
| (precursor form) | | LPKTEANWVNVISDLKKIEDLIQSMHIDATLYTESDVH |
| | | PSCKVTAMKCFLLELQVISLESGDASIHDTVENLIILAN |
| | | NSLSSNGNVTESGCKECEELEEKNIKEFLQSFVHIVQM |
| | | FINTS |
| Human IL- | 9 | ITCPPPMSVEHADIWVKSYSLYSRERYICNSGFKRKAGTS |
| 15Rα_sushi (short | | SLTECVLNKATNVAHWTTPSLKCIR |
| version 1) | | |
| Human IL- | 10 | TCPPPMSVEHADIWVKSYSLYSRERYICNSGFKRKAGTSS |
| 15Rα_sushi (short | | LTECVLNKATNVAHWTTPSLKCIR |
| version 2) | | |
| Human IL- | 11 | ITCPPPMSVEHADIWVKSYSLYSRERYICNSGFKRKAGTS |
| 15Rα_sushi (long | | SLTECVLNKATNVAHWTTPSLKCIRDPALVHQRPAPP |
| version) | | |

TABLE 5

| | SEQ ID NO. | Sequence |
|---|---|---|
| hIgG1 Fc(hole) | 12 | DKTHTCPPCPAPELLGGPSVFLFPPKPKDTLMISRTPEVTCVVV |
| T366S + L368A+ | | DVSHEDPEVKFNWYVDGVEVHNAKTKPREEQYNSTYRVVSV |
| Y407V | | LTVLHQDWLNGKEYKCKVSNKALPAPIEKTISKAKGQPREPQ |
| | | VYTLPPSREEMTKNQVSLSCAVKGFYPSDIAVEWESNGQPEN |
| | | NYKTTPPVLDSDGSFFLVSKLTVDKSRWQQGNVFSCSVMHEA |
| | | LHNHYTQKSLSLSPGK |
| hIgG1 Fc(knob) | 13 | DKTHTCPPCPAPELLGGPSVFLFPPKPKDTLMISRTPEVTCVVV |
| T366W | | DVSHEDPEVKFNWYVDGVEVHNAKTKPREEQYNSTYRVVSV |
| | | LTVLHQDWLNGKEYKCKVSNKALPAPIEKTISKAKGQPREPQ |
| | | VYTLPPSREEMTKNQVSLWCLVKGFYPSDIAVEWESNGQPEN |
| | | NYKTTPPVLDSDGSFFLYSKLTVDKSRWQQGNVFSCSVMHEA |
| | | LHNHYTQKSLSLSPGK |

TABLE 6

| Masking | SEQ | |
|---|---|---|
| polypeptide (MP) | ID NO. | Sequence |
| MP80 | 14 | GSPSGESSAPGSSSAESPGPGSESPSGSAPGESSPSGSAPGSPSG |
| | | ESSAPGSPESGSASPGSSSAESPGPGSPSGESSAP |
| MP163 | 15 | GSPSGESSAPGSSSAESPGPGSESPSGSAPGESSPSGSAPGSPSG |
| | | ESSAPGSPESGSASPGSSSAESPGPGSPSGESSAPGSSPSGESSAP |
| | | GSSSAESPGPGSESPSGSAPGESSPSGSAPGSPSGESSAPGSPES |
| | | GSASPGSSSAESPGPGSPSGESSAPGS |

TABLE 7

| Construct | Construct | | |
|---|---|---|---|
| name | description | SEQ ID NO. | Sequence |
| SB1902-C1 | hIgG | 16 | DKTHTCPPCPAPELLGGPSVFLFPPKPKDTLMISRTPEV |
| | Fc(hole)-lk1- | | |
| | IL15 | | TCVVVDVSHEDPEVKFNWYVDGVEVHNAKTKPREE |
| | | | QYNSTYRVVSVLTVLHQDWLNGKEYKCKVSNKALP |
| | | | APIEKTISKAKGQPREPQVYTLPPSREEMTKNQVSLSC |
| | | | AVKGFYPSDIAVEWESNGQPENNYKTTPPVLDSDGSF |
| | | | FLVSKLTVDKSRWQQGNVFSCSVMHEALHNHYTQKS |
| | | | 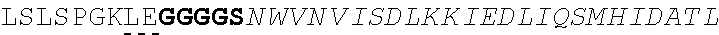 |
| | | | YTESDVHPSCKVTAMKCFLLELQVISLESGDASIHDTVEN |
| | | | LIILANNSLSSNGNVTESGCKECEELEEKNIKEFLQSFVHI |
| | | | VQMFINTS |
| | hIgG | 17 | DKTHTCPPCPAPELLGGPSVFLFPPKPKDTLMISRTPEV |
| | Fc(knob-lk5- | | |
| | IL15Rα_sushi | | TCVVVDVSHEDPEVKFNWYVDGVEVHNAKTKPREE |
| | | | QYNSTYRVVSVLTVLHQDWLNGKEYKCKVSNKALP |
| | | | APIEKTISKAKGQPREPQVYTLPPSREEMTKNQVSLWC |
| | | | LVKGFYPSDIAVEWESNGQPENNYKTTPPVLDSDGSF |
| | | | FLYSKLTVDKSRWQQGNVFSCSVMHEALHNHYTQKS |
| | | | 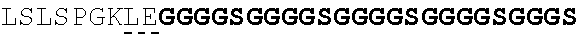 |
| | | | 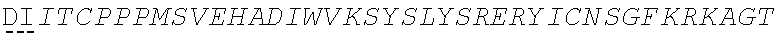 |
| | | | SSLTECVLNKATNVAHWTTPSLKCIR |
| SB1902- | hIgG | 16 | DKTHTCPPCPAPELLGGPSVFLFPPKPKDTLMISRTPEV |
| C1-variant1 | Fc(hole)-lk1- | | |
| | IL15 | | TCVVVDVSHEDPEVKFNWYVDGVEVHNAKTKPREE |
| | | | QYNSTYRVVSVLTVLHQDWLNGKEYKCKVSNKALP |
| | | | APIEKTISKAKGQPREPQVYTLPPSREEMTKNQVSLSC |
| | | | AVKGFYPSDIAVEWESNGQPENNYKTTPPVLDSDGSF |
| | | | FLVSKLTVDKSRWQQGNVFSCSVMHEALHNHYTQKS |
| | | | 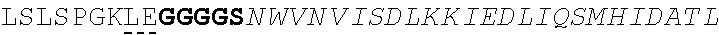 |
| | | | YTESDVHPSCKVTAMKCFLLELQVISLESGDASIHDTVEN |
| | | | LIILANNSLSSNGNVTESGCKECEELEEKNIKEFLQSFVHI |
| | | | VQMFINTS |
| | hIgG | 18 | DKTHTCPPCPAPELLGGPSVFLFPPKPKDTLMISRTPEV |
| | Fc(knob)-lk- | | |
| | IL15Rα_sushi | | TCVVVDVSHEDPEVKFNWYVDGVEVHNAKTKPREE |
| | | | QYNSTYRVVSVLTVLHQDWLNGKEYKCKVSNKALP |
| | | | APIEKTISKAKGQPREPQVYTLPPSREEMTKNQVSLWC |
| | | | LVKGFYPSDIAVEWESNGQPENNYKTTPPVLDSDGSF |
| | | | FLYSKLTVDKSRWQQGNVFSCSVMHEALHNHYTQKS |
| | | | LSLSPGKGSGTCPPPMSVEHADIWVKSYSLYSRERYICNS |
| | | | GFKRKAGTSSLTECVLNKATNVAHWTTPSLKCIR |
| SB1902-C2 | hIgG | 19 | DKTHTCPPCPAPELLGGPSVFLFPPKPKDTLMISRTPEV |
| | Fc(hole)-lk1- | | |
| | IL15-1k2- | | TCVVVDVSHEDPEVKFNWYVDGVEVHNAKTKPREE |
| | CM1-1k2- | | |
| | MP80 | | QYNSTYRVVSVLTVLHQDWLNGKEYKCKVSNKALP |
| | | | APIEKTISKAKGQPREPQVYTLPPSREEMTKNQVSLSC |
| | | | AVKGFYPSDIAVEWESNGQPENNYKTTPPVLDSDGSF |
| | | | FLVSKLTVDKSRWQQGNVFSCSVMHEALHNHYTQKS |
| | | | 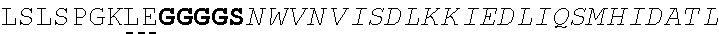 |
| | | | YTESDVHPSCKVTAMKCFLLELQVISLESGDASIHDTVEN |
| | | | LIILANNSLSSNGNVTESGCKECEELEEKNIKEFLQSFVHI |
| | | | 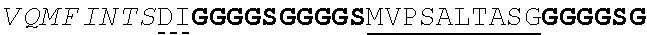 |
| | | | 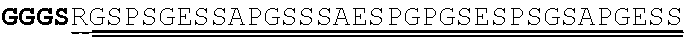 |
| | | | PSGSAPGSPSGESSAPGSPESGSASPGSSSAESPGPGSPS |
| | | | GESSAP |
| | hIgG | 20 | DKTHTCPPCPAPELLGGPSVFLFPPKPKDTLMISRTPEV |
| | Fc(knob)-lk5- | | |
| | IL15Rα_sushi | | TCVVVDVSHEDPEVKFNWYVDGVEVHNAKTKPREE |
| | | | QYNSTYRVVSVLTVLHQDWLNGKEYKCKVSNKALP |
| | | | APIEKTISKAKGQPREPQVYTLPPSREEMTKNQVSLWC |
| | | | LVKGFYPSDIAVEWESNGQPENNYKTTPPVLDSDGSF |
| | | | FLYSKLTVDKSRWQQGNVFSCSVMHEALHNHYTQKS |
| | | | 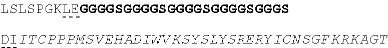 |
| | | | SSLTECVLNKATNVAHWTTPSLKCIR |
| SB1902- | hIgG | 19 | DKTHTCPPCPAPELLGGPSVFLFPPKPKDTLMISRTPEV |
| variant0 | Fc(hole)-lk1- | | |
| C2- | IL15-1k2- | | TCVVVDVSHEDPEVKFNWYVDGVEVHNAKTKPREE |
| | CM1-1k2- | | |
| | MP80 | | QYNSTYRVVSVLTVLHQDWLNGKEYKCKVSNKALP |
| | | | APIEKTISKAKGQPREPQVYTLPPSREEMTKNQVSLSC |
| | | | AVKGFYPSDIAVEWESNGQPENNYKTTPPVLDSDGSF |
| | | | FLVSKLTVDKSRWQQGNVFSCSVMHEALHNHYTQKS |
| | | | LSLSPGKLEGGGGSNWVNVISDLKKIEDLIQSMHIDATL |
| | | | YTESDVHPSCKVTAMKCFLLELQVISLESGDASIHDTVEN |
| | | | LIILANNSLSSNGNVTESGCKECEELEEKNIKEFLQSFVHI |
| | | | VQMFINTSDIGGGGSGGGGSMVPSALTASGGGGGSG |
| | | | GGGSRGSPSGESSAPGSSSAESPGPGSESPSGSAPGESS |
| | | | PSGSAPGSPSGESSAPGSPESGSASPGSSSAESPGPGSPS |
| | | | GESSAP |
| | hIgG | 18 | DKTHTCPPCPAPELLGGPSVFLFPPKPKDTLMISRTPEV |
| | Fc(knob)-lk- | | |
| | IL15Rα_sushi | | TCVVVDVSHEDPEVKFNWYVDGVEVHNAKTKPREE |
| | | | QYNSTYRVVSVLTVLHQDWLNGKEYKCKVSNKALP |
| | | | APIEKTISKAKGQPREPQVYTLPPSREEMTKNQVSLWC |
| | | | LVKGFYPSDIAVEWESNGQPENNYKTTPPVLDSDGSF |
| | | | FLYSKLTVDKSRWQQGNVFSCSVMHEALHNHYTQKS |
| | | | LSLSPGKGSGTCPPPMSVEHADIWVKSYSLYSRERYICNS |
| | | | GFKRKAGTSSLTECVLNKATNVAHWTTPSLKCIR |
| SB1902- | hIgG | 21 | DKTHTCPPCPAPELLGGPSVFLFPPKPKDTLMISRTPEV |
| C9-variant1 | Fc(hole)-lk5- | | |
| | IL15Rα_sushi | | TCVVVDVSHEDPEVKFNWYVDGVEVHNAKTKPREE |
| | | | QYNSTYRVVSVLTVLHQDWLNGKEYKCKVSNKALP |
| | | | APIEKTISKAKGQPREPQVYTLPPSREEMTKNQVSLSC |
| | | | AVKGFYPSDIAVEWESNGQPENNYKTTPPVLDSDGSF |
| | | | FLVSKLTVDKSRWQQGNVFSCSVMHEALHNHYTQKS |
| | | | 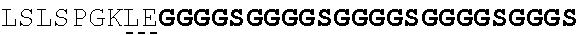 |
| | | | 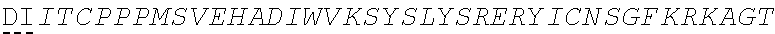 |
| | | | SSLTECVLNKATNVAHWTTPSLKCIR |
| | hIgG | 22 | DKTHTCPPCPAPELLGGPSVFLFPPKPKDTLMISRTPEV |
| | Fc(knob-lk1- | | |
| | IL15-1k2- | | TCVVVDVSHEDPEVKFNWYVDGVEVHNAKTKPREE |
| | CM1-1k2- | | |
| | MP163 | | QYNSTYRVVSVLTVLHQDWLNGKEYKCKVSNKALP |
| | | | APIEKTISKAKGQPREPQVYTLPPSREEMTKNQVSLWC |
| | | | LVKGFYPSDIAVEWESNGQPENNYKTTPPVLDSDGSF |
| | | | FLYSKLTVDKSRWQQGNVFSCSVMHEALHNHYTQKS |
| | | | 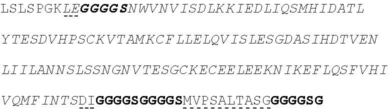 |
| | | | 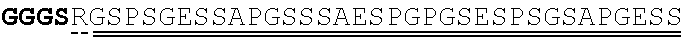 |
| | | | PSGSAPGSPSGESSAPGSPESGSASPGSSSAESPGPGSPS |
| | | | GESSAPGSSPSGESSAPGSSSAESPGPGSESPSGSAPGES |
| | | | SPSGSAPGSPSGESSAPGSPESGSASPGSSSAESPGPGSP |
| | | | SGESSAPGS |
| SB1902- | hIgG | 23 | DKTHTCPPCPAPELLGGPSVFLFPPKPKDTLMISRTPEV |
| C9-variant2 | Fc(hole)-lk3- | | |
| | IL15Rα_sushi | | TCVVVDVSHEDPEVKFNWYVDGVEVHNAKTKPREE |
| | | | QYNSTYRVVSVLTVLHQDWLNGKEYKCKVSNKALP |
| | | | APIEKTISKAKGQPREPQVYTLPPSREEMTKNQVSLSC |
| | | | AVKGFYPSDIAVEWESNGQPENNYKTTPPVLDSDGSF |
| | | | FLVSKLTVDKSRWQQGNVFSCSVMHEALHNHYTQKS |
| | | | 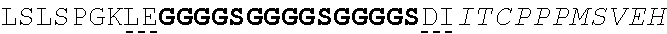 |
| | | | ADIWVKSYSLYSRERYICNSGFKRKAGTSSLTECVLNKATN |
| | | | VAHWTTPSLKCIR |
| | hIgG | 22 | DKTHTCPPCPAPELLGGPSVFLFPPKPKDTLMISRTPEV |
| | Fc(knob)-lk1- | | |
| | IL15-1k2- | | TCVVVDVSHEDPEVKFNWYVDGVEVHNAKTKPREE |
| | CM1-1k2- | | |
| | MP163 | | QYNSTYRVVSVLTVLHQDWLNGKEYKCKVSNKALP |
| | | | APIEKTISKAKGQPREPQVYTLPPSREEMTKNQVSLWC |
| | | | LVKGFYPSDIAVEWESNGQPENNYKTTPPVLDSDGSF |
| | | | FLYSKLTVDKSRWQQGNVFSCSVMHEALHNHYTQKS |
| | | | 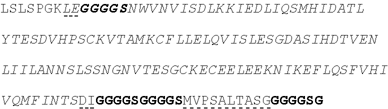 |
| | | | 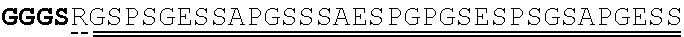 |
| | | | PSGSAPGSPSGESSAPGSPESGSASPGSSSAESPGPGSPS |
| | | | GESSAPGSSPSGESSAPGSSSAESPGPGSESPSGSAPGES |
| | | | SPSGSAPGSPSGESSAPGSPESGSASPGSSSAESPGPGSP |
| | | | SGESSAPGS |
| SB1902- | hIgG | 24 | DKTHTCPPCPAPELLGGPSVFLFPPKPKDTLMISRTPEV |
| C9-variant3 | Fc(hole)-lk1- | | |
| | IL15Rα_sushi | | TCVVVDVSHEDPEVKFNWYVDGVEVHNAKTKPREE |
| | | | QYNSTYRVVSVLTVLHQDWLNGKEYKCKVSNKALP |
| | | | APIEKTISKAKGQPREPQVYTLPPSREEMTKNQVSLSC |
| | | | AVKGFYPSDIAVEWESNGQPENNYKTTPPVLDSDGSF |
| | | | FLVSKLTVDKSRWQQGNVFSCSVMHEALHNHYTQKS |
| | | | 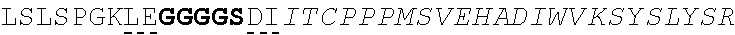 |
| | | | ERYICNSGFKRKAGTSSLTECVLNKATNVAHWTTPSLKCI |
| | | | R |
| | hIgG | 22 | DKTHTCPPCPAPELLGGPSVFLFPPKPKDTLMISRTPEV |
| | Fc(knob)-lk1- | | |
| | IL15-1k2- | | TCVVVDVSHEDPEVKFNWYVDGVEVHNAKTKPREE |
| | CM1-Ik2- | | |
| | MP163 | | QYNSTYRVVSVLTVLHQDWLNGKEYKCKVSNKALP |
| | | | APIEKTISKAKGQPREPQVYTLPPSREEMTKNQVSLWC |
| | | | LVKGFYPSDIAVEWESNGQPENNYKTTPPVLDSDGSF |
| | | | FLYSKLTVDKSRWQQGNVFSCSVMHEALHNHYTQKS |
| | | | 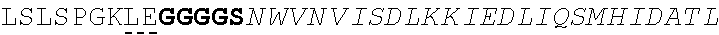 |
| | | | YTESDVHPSCKVTAMKCFLLELQVISLESGDASIHDTVEN |
| | | | LIILANNSLSSNGNVTESGCKECEELEEKNIKEFLQSFVHI |
| | | | 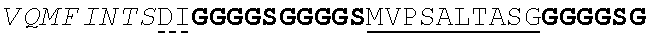 |
| | | | PSGSAPGSPSGESSAPGSPESGSASPGSSSAESPGPGSPS |
| | | | GESSAPGSSPSGESSAPGSSSAESPGPGSESPSGSAPGES |
| | | | SPSGSAPGSPSGESSAPGSPESGSASPGSSSAESPGPGSP |
| | | | SGESSAPGS |

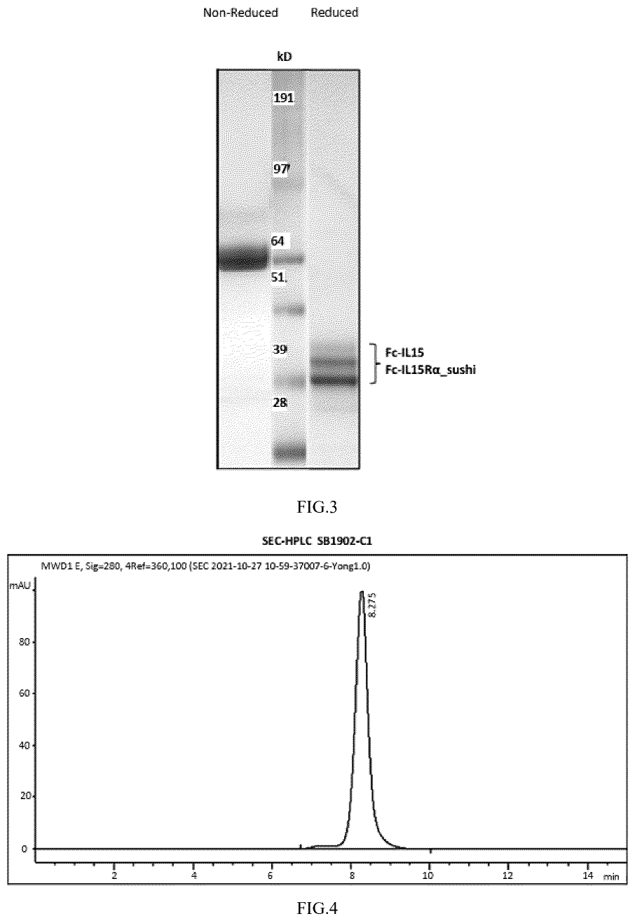

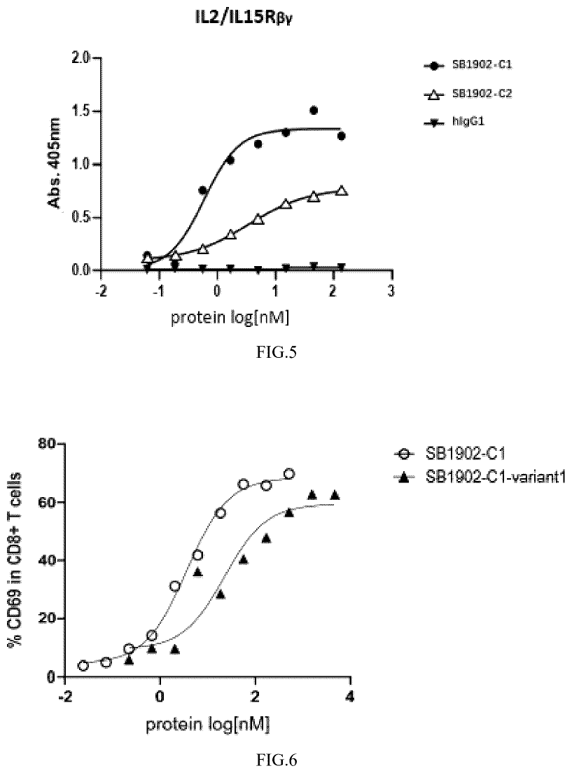

Expression and purification: the vectors coding the Fc-IL-15/Fc-IL-15Rα_sushi heterodimeric proteins and IL-15 prodrugs were transiently transfected, and proteins were expressed in Expi293 cells (Thermo Fisher Scientific, Waltham, MA, USA) according to the manufacturer's protocol. The culture supernatant media were clarified by centrifugation and 0.2 μm membrane filtration. The exemplary IL-15 prodrugs (SB1902-C2, SB1902-C2-variant0, SB1902-C9 variant1, SB1902-C9-variant2 and SB1902-C9-variant3), the exemplary Fc-IL-15/Fc-IL-15Rα_sushi heterodimeric proteins (SB1902-C1 and SB1902-C1-variant1) were purified by a two-step purification process comprising a precast MabSelect SuRe pcc column (Cytiva lifescience, Cat number17549112) and size-exclusion chromatography (Superdex200, Cytiva, USA), according to the manufacturer's protocol. The purity of the purified molecules was analyzed by SDS-PAGE in the presence and absence of a reducing agent and SEC-HPLC (some data not shown). The SDS-PAGE and SEC-HPLC analysis result of SB1902-C1 was shown in FIG. 3 and FIG. 4 respectively, this exemplary data demonstrated that Fc-IL-15/Fc-IL-15Rα_sushi heterodimeric protein was successfully produced, and showed good purity and homogeneity.

### Example 2: Receptor Binding Affinity

The binding affinity of the Fc-IL-15/Fc-IL-15Rα_sushi heterodimeric protein and IL-15 prodrug to IL-2/IL-15Rβγ-Fc fusions was assessed by ELISA. The ELISA plate was coated with recombinant IL-15Rβγ-Fc at 1 μg/ml at 4° C. overnight. After blocking with 1% BSA in PBS and plate wash, a serial diluted SB1902-C1 or SB1902-C2 was loaded to the plate and incubated for 2 hours. A Human IgG1 isotype antibody MPOC21 abbreviated as hIgG1 in FIG. 5 (see Hamlyn P H, Gait M J, Milstein C. (1981) Complete sequence of an immunoglobulin mRNA using specific priming and the dideoxynucleotide method of RNA sequencing. Nucleic Acids Res. 9(18):4485-4494) was used as a negative control. After plate wash, an anti-human IgG-Fc-AP conjugated antibody at 1:2000 dilution was added to the individual well of the plate and incubated for 45 min. After plate wash, the AP substrate pNPP (Thermo, USA) was added and the optical densities were obtained at 405 nm with a spectrophotometer (BioTec, USA). The data were analyzed and plotted with GraphPad Prism8 software.

As shown in FIG. 5, the Fc-IL-15/Fc-IL-15Rα_sushi heterodimeric protein SB1902-C1 and prodrug from thereof SB1902-C2 both exhibited binding affinity to IL-15Rβγ, indicating the proteins have the potential to exhibit IL-15 activity, and the prodrug SB1902-C2 showed lower binding affinity to IL-15Rβγ than SB1902-C1. Other Fc-IL-15/Fc-IL-15Rα_sushi heterodimeric proteins or prodrug form thereof also exhibited binding to the IL-2/IL-15Rβγ receptor (data not shown).

### Example 3: In Vitro Functional Experiments: CD8+ T Cell Activation Assay

It was well documented that IL-15 promotes CD8+ memory T, natural killer (NK), and NKT cells proliferation, survival, and homeostasis. IL-15 causes T cell activation indicated by upregulation of the membrane surface expression of CD69. CD69 was an early activation marker of T cells. As measured by the percentage of CD69 surface expression to reflect the percentage of CD8+ T cell activation. The FEc-IL-15/Fc-IL-15Rα_sushi heterodimeric proteins and IL-15 prodrugs in the various formats as described above were tested in the CD8+ T cell activation assay.

This assay was performed essentially as follows: human PBMC (Stem Cell Technologies, Catalog number 70500) were plated with a cell number of 2×10⁵/well in 96 well round-bottom cell culture plate in RPMI-1640 medium supplemented with 10% FBS, 1% penicillin, and streptomycin. The Fc-IL-15/Fc-IL-15Rα_sushi heterodimeric proteins and IL-15 prodrugs were 3-times titrated with medium and 5 μL added to the wells. Each concentration was repeated in triplicate, and blank wells (added with only medium) were used as a blank control. Cell plates were cultured in the incubator for 3 days. Then the cells were centrifuged and the cell pellet was stained with anti-CD3 Ab (BioLegend, cat #317306), anti-CD8 Ab (BioLegend, cat #344722), and anti-CD69 Ab (BioLegend, cat #310906) in FACS buffer for 30 minutes. They were washed twice with FACS buffer and acquired by flow cytometry with Attune (ThermoFisher Scientific). Data were analyzed with FlowJo software. The percentage of CD69+ cells in CD8+ T cells was plotted with Graphpad Prism 8 software.

To compare the effect of the different length of linkers between Fc and IL-15Rα_sushi on IL-15 activity, the biological activity was determined by measuring the induction of CD69 expression on human CD8+ T cells in an assay based on ex vivo human PBMC FACS as described above.

In group 1 of SB1902-C1 construct, the linker between Fc and IL-15Rα_sushi was a GS-rich flexible linker with a length of 25 amino acid (GGGGS)₅ (SEQ ID NO: 5), and in group 1 of SB1902-C1-variant1 construct, the linker between Fc and IL-15Rα_sushi was a GS-rich flexible linker with a length of 3 amino acid GSG (SEQ ID NO: 1). The only difference between SB1902-C1 and SB1902-C1-variant1 is the linker between Fc and IL-15Rα_sushi. As shown in FIG. 6, SB1902-C1 with the linker “lk5” (GGGGS)₅ between Fc and IL-15Rα_sushi had better activity than the SB1902-C1-variant1 with the linker “lk” (GSG) between Fc and IL-15Rα_sushi in inducing CD69 expression in CD8+ T Cell Activation Assay.

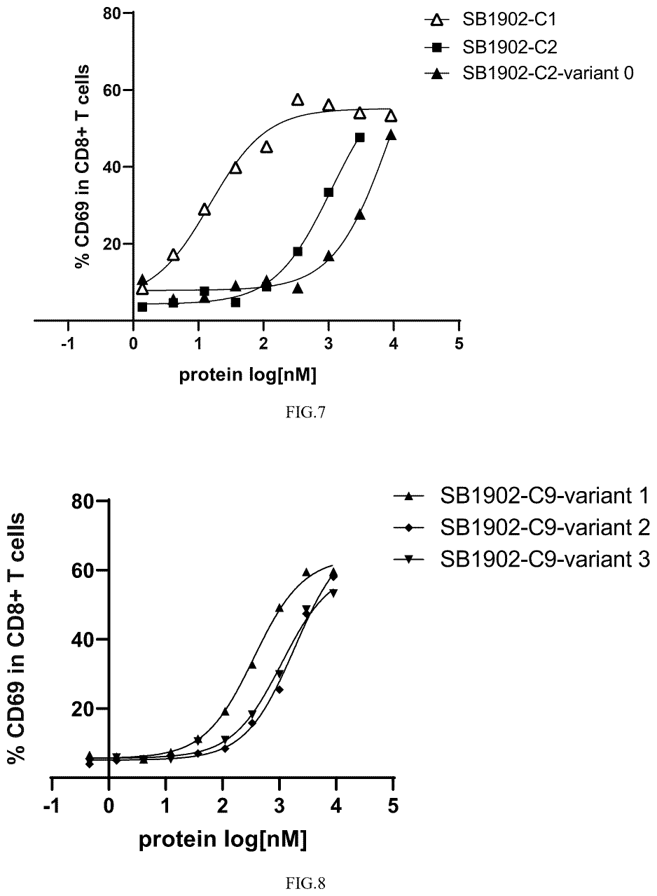

In group 2 of SB1902-C2 construct, the linker between Fc and IL-15Rα_sushi was a GS-rich flexible linker with a length of 25 amino acid (GGGGS)₅, (SEQ ID NO: 5), and in group 2 of SB1902-C2-variant0 construct, the linker between Fc and IL-15Rα_sushi was a GS-rich flexible linker with a length of 3 amino acid GSG (SEQ ID NO: 1). The only difference between SB1902-C2 and SB1902-C2-variant0 is the linker between Fc and IL-15Rα_sushi. As shown in FIG. 7, SB1902-C2 with the linker “lk5” (GGGGS)₅ between Fc and IL-15Rα_sushi had better activity than the SB1902-C2-variant0 with the linker “lk” (GSG) between Fc and IL-15Rα_sushi in inducing CD69 expression in CD8+ T Cell Activation Assay.

In group 3 of SB1902-C9-variant1 construct, the linker between Fc and IL-15Rα_sushi was a GS-rich flexible linker with a length of 25 amino acid (GGGGS)₅, (SEQ ID NO: 5), while in group 3 of SB1902-C9-variant2 construct, the linker between Fc and IL-15Rα_sushi was a GS-rich flexible linker with a length of 15 amino acid (GGGGS)₃ (SEQ ID NO: 4), and in group 3 of SB1902-C9-variant3 construct, the linker between Fc and IL-15Rα_sushi was a GS-rich flexible linker with a length of 5 amino acid (GGGGS)₁, (SEQ ID NO: 2). The only difference among SB1902-C9-variant1, SB1902-C9-variant2 and SB1902-C9-variant3 is the linker between Fc and IL-15Rα_sushi. As shown in FIG. 8, SB1902-C9-variant2 with the linker “lk3” (GGGGS)₃ between Fc and IL-15Rα_sushi and SB1902-C9-variant3 with the linker “lk1” (GGGGS)₁ between Fc and IL-15Rα_sushi showed comparable activity with each other, however, the SB1902-C9-variant1 with the linker “lk5” (GGGGS)₅ between Fc and IL-15Rα_sushi had better activity than the SB1902-C9-variant2 and SB1902-C9-variant3 in inducing CD69 expression in CD8+ T Cell Activation Assay.

In combination of FIGS. 6, 7 and 8, the results indicated that the length of the peptide linker connecting Fc and IL-15 Rα_sushi does affect the biological activity of the IL-15 in Fc-IL-15/Fc-IL-15Rα_sushi heterodimeric proteins or prodrug form thereof within a certain range, when the length of the linker was less than 15 amino acids, the length of the linker has little impact on IL-15 activity, while when the length of the linker was more than 15 amino acids, and less than 25 amino acids, the longer the linker length, the better the activity of Fc-IL-15/Fc-IL-15Rα_sushi heterodimeric protein and IL-15 in prodrugs.

### Example 4: In Vivo Tumor Models to Evaluate Activity of IL-15/IL-15Rα_Sushi-Fc Heterodimeric Protein

The ability of the Fc-IL-15/Fc-IL-15Rα_sushi heterodimeric protein to promote tumor eradication and inhibit metastasis is assessed in vivo using the mouse WEHI-164 tumor model. A human IgG1 isotype antibody MOPC 21 was used as a control in this experiment.

### A. In Vivo Activity of Prodrug in WEHI-164 Subcutaneous Tumor Model

Animals and husbandry: Female mice (7-9 weeks of age) were used in the studies. The animals were fed irradiated Harlan 2918.15 Rodent Diet and water ad libitum. Animals were ear-tagged for identification purposes and shaved on the left dorsal flank area in preparation for cell implantation. Animals were housed in Innovive disposable ventilated caging with corn cob bedding at 60 complete air changes per hour. The environment was controlled to a temperature range of 70°±2° F. and a humidity range of 30-70%. All procedures carried out in this experiment were conducted by skilled personnel in compliance with all the laws, regulations, and guidelines of the National Institutes of Health (NIH) and with the approval of Biomere's Animal Care and Use Committee (Richmond, CA).

Cell preparation and implantation: WEHI-164 cells were obtained from ATCC (CAT #: CRL-1751™). WEHI-164 cells were cultured and expanded in Dulbecco's Modified Eagles Medium (DMEM) with 2 mM L-glutamine, 10% fetal bovine serum (FBS), and 1% 100×Penicillin/Streptomycin (PS). The growth environment was maintained in an incubator with a 5% CO₂ atmosphere at 37° C. When the expansion was complete, the cells were trypsinized using a 0.25% trypsin-EDTA solution. The cells were then washed and counted. Pre-implantation cell viability was >95%. The cells were resuspended in Dulbecco's Phosphate Buffered Saline (DPBS). Test animals were sterilized at the implantation site with an alcohol prep pad and were implanted subcutaneously on Day 0 in 0.1 mL using a 25-gauge needle and 1 mL syringe.

Measurements and treatment: Tumors were allowed to grow at the range of 70-150 mm³ and were then randomized into study groups. Mice were distributed to ensure that the mean body weights for all groups were within 10% of the overall mean tumor burden for the study population. Mice were intravenously injected twice weekly with a dose of 3 mg/kg of human IgG1 isotype antibody or the Fc-IL-15/Fc-IL-15Rα_sushi heterodimeric protein for 2 weeks and tumor volumes were monitored.

Assessment of side effects: All animals were observed for clinical signs of distress or toxicity at least once daily. Animals were weighed once per week. If an individual animal showed overt signs of distress or 15% body weight loss, the individual animal was weighed daily. Animals were euthanized if bodyweight loss was in excess of 20% or other clinical signs warranted euthanasia. Individual animals were euthanized when their tumor volume reached or exceeded 2000 mm³.

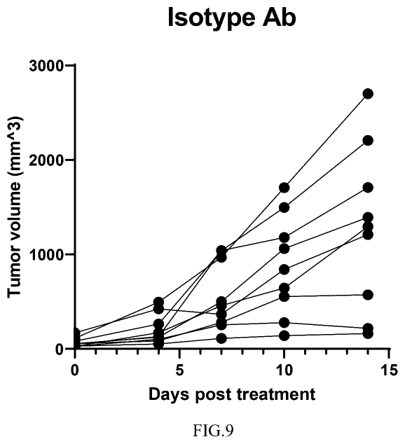

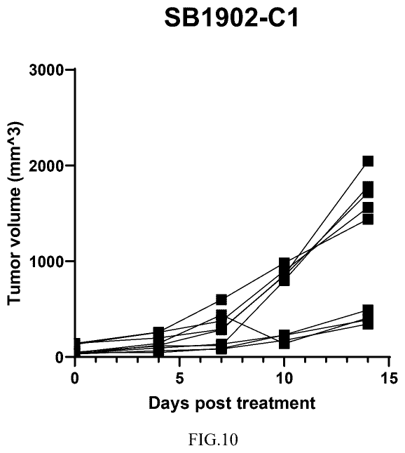

Results: SB1902-C1 was used as an example. As shown in FIGS. 9-10, animals with WEHI-164 tumor were treated with human IgG1 isotype control antibody or SB1902-C1. All animals in the study did not show overt signs of systemic toxicity throughout the treatment course. The tumor growth in SB1902-C1 treated animals (FIG. 10) was inhibited compared to the tumor growth in animals treated with isotype control antibody (FIG. 9). This result demonstrates that Fc-IL-15/Fc-IL-15Rα_sushi heterodimeric protein can function in the tumor bearing mice.

The in vivo assays for other heterodimeric proteins or prodrug forms thereof were also performed and the effect of linker length between Fc and IL-15Rα_sushi in other Fc-IL-15/Fc-IL-15Rα_sushi heterodimeric proteins or prodrug forms thereof has been further verified in vivo tumor model, which were consistent with the trend proved in CD8+ T Cell Activation Assay (data not shown).

1-23. (canceled)

24. A method of improving the activity of a fusion protein or a heterodimeric protein comprising IL-15Rα or a functional fragment thereof and Fc domain, wherein, the peptide linker connecting the IL-15Rα or a functional fragment thereof and the Fc domain was set and the length of the peptide linker was more than 15 and less than 40 amino acid residues.

25. The method according to claim 24, wherein the peptide linker comprises 20 to 25 amino acid residues.

26. The method according to claim 24, wherein the peptide linker comprises 25 amino acid residues.

27-34. (canceled)

35. The method according to claim 24, wherein the IL-15Rα or a functional fragment thereof is linked to the N-terminus or C-terminus of the Fc domain.

36. The method according to claim 24, wherein the IL-15Rα or a functional fragment thereof is selected from an extracellular region of IL-15Rα or a sushi domain or functional analogs.

37. The method according to claim 24, wherein the IL-15Rα or a functional fragment comprises the amino acid sequence of any one of SEQ ID NOs: 9-11, or a variant thereof having at least about 90% sequence identity to the amino acid sequence of any one of SEQ ID NOs: 9-11.

38. The method according to claim 24, wherein the fusion protein or heterodimeric protein further comprises an IL-15 cytokine.

39. The method according to claim 38, wherein the IL-15 cytokine comprises the amino acid sequence of any one of SEQ ID NOs: 7-8, or a variant thereof having at least about 90% sequence identity to the amino acid sequence of any one of SEQ ID NOs: 7-8.

40. The method according to claim 38, wherein the heterodimeric protein comprises two monomers, wherein one monomer comprises IL-15Rα or a functional fragment and Fc domain, and the other monomer comprises an IL-15 cytokine and a Fc domain.

41. The method according to claim 40, wherein the IL-15Rα or a functional fragment thereof and the IL-15 cytokine is linked to the C-terminus of the Fc domain.

42. The method according to claim 41, wherein the Fc domain is selected from the group consisting of a human IgG1 Fc domain, a human IgG2 Fc domain, a human IgG3 Fc domain, a human IgG4 Fc domain, an IgA Fc domain, an IgD Fc domain, an IgE Fc domain, and an IgM Fc domain.

43. The method according to claim 41, wherein the Fc domain is a human IgG1 Fc domain having L234A and L235A mutations, according to EU Numbering system.

44. The method according to claim 41, wherein the Fc domains comprise knobs-into-holes mutations (Fc knob and Fc hole), wherein the Fc knob comprises a T366W mutation in the Fc domain, and the Fc hole comprises T366S, L368A, and Y407V mutations in the Fc domain, according to EU Numbering system.

45. The method according to claim 44, wherein the Fc knob further comprises S354C mutation, and the Fc hole further comprises Y349C mutation, according to EU Numbering system.

46. The method according to claim 24, wherein the peptide linker consists of G and S amino acid residues.

47. The method according to claim 24, wherein the peptide linker comprises the amino acid sequence of any one of SEQ ID NOs: 4-5, or a variant thereof having at least about 90% sequence identity to the amino acid sequence of any one of SEQ ID NOs: 4-5.

48. The method according to claim 38, wherein the fusion protein or heterodimeric protein further comprises cleavable moiety (CM) and masking polypeptide (MP), and wherein the masking polypeptide (MP) is linked to the IL-15 cytokine through the cleavable moiety (CM).

49. The method according to claim 48, wherein the masking polypeptide (MP) comprises the amino acid sequence of any one of SEQ ID NOs: 14-15, or a variant thereof having at least about 90% sequence identity to the amino acid sequence of any one of SEQ ID NOs: 14-15.

50. The method according to claim 48, wherein the cleavable moiety (CM) comprises the amino acid sequence of SEQ ID NO: 6, or a variant thereof having at least about 90% sequence identity to the amino acid sequence of SEQ ID NO: 6.

51. The method according to claim 40, wherein the fusion protein or a heterodimeric protein comprises two polypeptide chains, wherein, one polypeptide chain comprises the amino acid sequence of SEQ ID NO: 16, or a variant thereof having at least about 80% sequence identity to the amino acid sequence of SEQ ID NO: 16; and the other polypeptide chain comprises the amino acid sequence of SEQ ID NO: 17, or a variant thereof having at least about 80% sequence identity to the amino acid sequence of SEQ ID NO: 17.
